SEQUENCE LISTING (104 sequences)

SEQ ID NO: 1 - DNA, 354 bp, synthetic construct
  misc_feature 1..354: note = synthetic
  source 1..354: mol_type = other DNA
caggtgcagctggtggagtctgggggaggcgtggtccagcctgggaggtccctgagactctcctgtgcagcctctggattcagcttcagtagctttggcatgcactgggtccgccaggctccaggcaagggactggagtgggtgtcagttatatcatatgatggaattaatacatactatacagactccgtgaagggccgattcaccatctccagagacaattccaagaacacgctgtatctgcaaatgaacggcctgagagctgaggacacggctctttattactgtgtgcaagggtcaattggaaccgtttttgaatactggggccagggaaccctggtcaccgtctcctca

SEQ ID NO: 2 - protein, 118 aa, synthetic construct
  REGION 1..118: note = synthetic
QVQLVESGGGVVQPGRSLRLSCAASGFSFSSFGMHWVRQAPGKGLEWVSVISYDGINTYYTDSVKGRFTISRDNSKNTLYLQMNGLRAEDTALYYCVQGSIGTVFEYWGQGTLVTVSS

SEQ ID NO: 3 - DNA, 24 bp, synthetic construct
  misc_feature 1..24: note = synthetic
  source 1..24: mol_type = other DNA
ggattcagcttcagtagctttggc

SEQ ID NO: 4 - protein, 8 aa, synthetic construct
  REGION 1..8: note = synthetic
GFSFSSFG

SEQ ID NO: 5 - DNA, 24 bp, synthetic construct
  misc_feature 1..24: note = synthetic
  source 1..24: mol_type = other DNA
atatcatatgatggaattaataca

SEQ ID NO: 6 - protein, 8 aa, synthetic construct
  REGION 1..8: note = synthetic
ISYDGINT

SEQ ID NO: 7 - DNA, 33 bp, synthetic construct
  misc_feature 1..33: note = synthetic
  source 1..33: mol_type = other DNA
gtgcaagggtcaattggaaccgtttttgaatac

SEQ ID NO: 8 - protein, 11 aa, synthetic construct
  REGION 1..11: note = synthetic
VQGSIGTVFEY

SEQ ID NO: 9 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcgagtcagggcattagcaattatttagcctggtatcagcagaaaccagggaaagttcctaaactcctgatctatgctgcatccactttacaatcaggggtcccatctcggttcggtggcagtggatctgggacagatttcactctcaccatcagcagcctgcagcctgaagatgttgcaacttattactgtcaaaagtataccagtgccccattcactttcggccctgggaccaaagtggatatcaaa

SEQ ID NO: 10 - protein, 107 aa, synthetic construct
  REGION 1..107: note = synthetic
DIQMTQSPSSLSASVGDRVTITCRASQGISNYLAWYQQKPGKVPKLLIYAASTLQSGVPSRFGGSGSGTDFTLTISSLQPEDVATYYCQKYTSAPFTFGPGTKVDIK

SEQ ID NO: 11 - DNA, 18 bp, synthetic construct
  misc_feature 1..18: note = synthetic
  source 1..18: mol_type = other DNA
cagggcattagcaattat

SEQ ID NO: 12 - protein, 6 aa, synthetic construct
  REGION 1..6: note = synthetic
QGISNY

SEQ ID NO: 13-14 - skipped (2 entries)

SEQ ID NO: 15 - DNA, 27 bp, synthetic construct
  misc_feature 1..27: note = synthetic
  source 1..27: mol_type = other DNA
caaaagtataccagtgccccattcact

SEQ ID NO: 16 - protein, 9 aa, synthetic construct
  REGION 1..9: note = synthetic
QKYTSAPFT

SEQ ID NO: 17 - DNA, 366 bp, synthetic construct
  misc_feature 1..366: note = synthetic
  source 1..366: mol_type = other DNA
gaggtgcagctggtggagtctgggggaggcttggtccagcctggggggtccctaaaactctcctgtacagcctctgggttcaccttcagtggctctgttattcactgggtccgccaggcttccgggaaagggctggagtggattggccgtattagaaacaaggctaacagttacgcgacagcatatggtgcgtcggtgacaggcaggttcaccatctccagagatgattcaaagaacacggcgtatctgcaaatgaacagcctgaaaaccgaggacacggccgtttattactgtactttcccgggtgtagtgggacgaggaggttttgactactggggccagggcaccctggtcaccgtctcctca

SEQ ID NO: 18 - protein, 122 aa, synthetic construct
  REGION 1..122: note = synthetic
EVQLVESGGGLVQPGGSLKLSCTASGFTFSGSVIHWVRQASGKGLEWIGRIRNKANSYATAYGASVTGRFTISRDDSKNTAYLQMNSLKTEDTAVYYCTFPGVVGRGGFDYWGQGTLVTVSS

SEQ ID NO: 19 - DNA, 24 bp, synthetic construct
  misc_feature 1..24: note = synthetic
  source 1..24: mol_type = other DNA
gggttcaccttcagtggctctgtt

SEQ ID NO: 20 - protein, 8 aa, synthetic construct
  REGION 1..8: note = synthetic
GFTFSGSV

SEQ ID NO: 21 - DNA, 30 bp, synthetic construct
  misc_feature 1..30: note = synthetic
  source 1..30: mol_type = other DNA
attagaaacaaggctaacagttacgcgaca

SEQ ID NO: 22 - protein, 10 aa, synthetic construct
  REGION 1..10: note = synthetic
IRNKANSYAT

SEQ ID NO: 23 - DNA, 39 bp, synthetic construct
  misc_feature 1..39: note = synthetic
  source 1..39: mol_type = other DNA
actttcccgggtgtagtgggacgaggaggttttgactac

SEQ ID NO: 24 - protein, 13 aa, synthetic construct
  REGION 1..13: note = synthetic
TFPGVVGRGGFDY

SEQ ID NO: 25 - DNA, 339 bp, synthetic construct
  misc_feature 1..339: note = synthetic
  source 1..339: mol_type = other DNA
gacatcgtgatgacccagtctccagactccctggctgtgtctctgggcgagggggccaccatcaactgcatgtccagccagagtgttttattcagctccaacaataagaactacttagcttggtaccaacagaaaccaggacagcctcctaagttgctcttttactgggcatctacccgggaatccggggtccctgaccgattcggtggcagcgggtctgggacagatttctctctcaccatcaacagcctgcagactgaagatgtggcagtttattactgtctccaatattatagtattccgtggacgttcggccaagggaccaaggtggaaatcaaa

SEQ ID NO: 26 - protein, 113 aa, synthetic construct
  REGION 1..113: note = synthetic
DIVMTQSPDSLAVSLGEGATINCMSSQSVLFSSNNKNYLAWYQQKPGQPPKLLFYWASTRESGVPDRFGGSGSGTDFSLTINSLQTEDVAVYYCLQYYSIPWTFGQGTKVEIK

SEQ ID NO: 27 - DNA, 36 bp, synthetic construct
  misc_feature 1..36: note = synthetic
  source 1..36: mol_type = other DNA
cagagtgttttattcagctccaacaataagaactac

SEQ ID NO: 28 - protein, 12 aa, synthetic construct
  REGION 1..12: note = synthetic
QSVLFSSNNKNY

SEQ ID NO: 29-30 - skipped (2 entries)

SEQ ID NO: 31 - DNA, 27 bp, synthetic construct
  misc_feature 1..27: note = synthetic
  source 1..27: mol_type = other DNA
ctccaatattatagtattccgtggacg

SEQ ID NO: 32 - protein, 9 aa, synthetic construct
  REGION 1..9: note = synthetic
LQYYSIPWT

SEQ ID NO: 33 - DNA, 381 bp, synthetic construct
  misc_feature 1..381: note = synthetic
  source 1..381: mol_type = other DNA
gaggtgcagctggtggagtctgggggaggcttggtacagcctggagggtccctgacactctcctgtgcagcctctggattcaatttccgtgattatgaaatgatctgggtccgccagactccagggaaggggctggagtggatttcatacattagtaatagtggttataccatatactacgcagactctgtgaagggccgattcaccatctccagagacaacgccaggaactcaatatatctgcaagtgaacagcctgagagccgaggacacggctgtttattactgttcgagacgtactactatgattcggggcattagggcgtactactattacggtctggacgtctggggccaagggaccacggtcaccgtctcctca

SEQ ID NO: 34 - protein, 127 aa, synthetic construct
  REGION 1..127: note = synthetic
EVQLVESGGGLVQPGGSLTLSCAASGFNFRDYEMIWVRQTPGKGLEWISYISNSGYTIYYADSVKGRFTISRDNARNSIYLQVNSLRAEDTAVYYCSRRTTMIRGIRAYYYYGLDVWGQGTTVTVSS

SEQ ID NO: 35 - DNA, 24 bp, synthetic construct
  misc_feature 1..24: note = synthetic
  source 1..24: mol_type = other DNA
ggattcaatttccgtgattatgaa

SEQ ID NO: 36 - protein, 8 aa, synthetic construct
  REGION 1..8: note = synthetic
GFNFRDYE

SEQ ID NO: 37 - DNA, 24 bp, synthetic construct
  misc_feature 1..24: note = synthetic
  source 1..24: mol_type = other DNA
attagtaatagtggttataccata

SEQ ID NO: 38 - protein, 8 aa, synthetic construct
  REGION 1..8: note = synthetic
ISNSGYTI

SEQ ID NO: 39 - DNA, 60 bp, synthetic construct
  misc_feature 1..60: note = synthetic
  source 1..60: mol_type = other DNA
tcgagacgtactactatgattcggggcattagggcgtactactattacggtctggacgtc

SEQ ID NO: 40 - protein, 20 aa, synthetic construct
  REGION 1..20: note = synthetic
SRRTTMIRGIRAYYYYGLDV

SEQ ID NO: 41 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 42 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 43 - DNA, 18 bp, synthetic construct
  misc_feature 1..18: note = synthetic
  source 1..18: mol_type = other DNA
cagagcattagcagctat

SEQ ID NO: 44 - protein, 6 aa, synthetic construct
  REGION 1..6: note = synthetic
QSISSY

SEQ ID NO: 45-46 - skipped (2 entries)

SEQ ID NO: 47 - DNA, 30 bp, synthetic construct
  misc_feature 1..30: note = synthetic
  source 1..30: mol_type = other DNA
caacagagttacagtacccctccgatcacc

SEQ ID NO: 48 - protein, 10 aa, synthetic construct
  REGION 1..10: note = synthetic
QQSYSTPPIT

SEQ ID NO: 49 - protein, 123 aa, synthetic construct
  REGION 1..123: note = M2aM14173N HCVR
NVSLVESGGGSVKPGGSLQLSCAASGFTFSSSAMSWIRQTPEMRLEWVATISDRGSYTYYPDSVEGRFTISRDNAKNILYLQMSHLMSEDTAMYYCARGKGEKAYYTAFFAYWGQGSLVTVSA

SEQ ID NO: 50 - protein, 8 aa, synthetic construct
  REGION 1..8: note = M2aM14173N HCDR1
GFTFSSSA

SEQ ID NO: 51 - protein, 8 aa, synthetic construct
  REGION 1..8: note = M2aM14173N HCDR2
ISDRGSYT

SEQ ID NO: 52 - protein, 16 aa, synthetic construct
  REGION 1..16: note = M2aM14173N HCDR3
ARGKGEKAYYTAFFAY

SEQ ID NO: 53 - protein, 107 aa, synthetic construct
  REGION 1..107: note = M2aM14173N LCVR
DIQMTQSPASLSASVGETVTITCRASENIYSYLAWYQQKPGKSPHLLVHNAETLAEGVPSRFSGSGSGPQFSLKINSLQPGDSGTYYCQHHYSSPWTFGGGTKLEIK

SEQ ID NO: 54 - protein, 6 aa, synthetic construct
  REGION 1..6: note = M2aM14173N LCDR1
ENIYSY

SEQ ID NO: 55 - skipped

SEQ ID NO: 56 - protein, 9 aa, synthetic construct
  REGION 1..9: note = M2aM14173N LCDR3
QHHYSSPWT

SEQ ID NO: 57 - protein, 330 aa, synthetic construct
  REGION 1..330: note = Mouse IgG2a
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 58 - protein, 107 aa, synthetic construct
  REGION 1..107: note = Constant LC
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 59 - protein, 119 aa, synthetic construct
  REGION 1..119: note = M2aM14178N HCVR
EVQLVESGGGLVKPGGSLKLSCTASGFTFSSYAMSWVRQTPEKRLEWVANISDGGTYTYYPDNIKGRFTISRDNAKNNLYLQMSHLESEDTAMYYCIRGDVYYVRFAYWGQGTLVTVSA

SEQ ID NO: 60 - protein, 8 aa, synthetic construct
  REGION 1..8: note = M2aM14178N HCDR1
GFTFSSYA

SEQ ID NO: 61 - protein, 8 aa, synthetic construct
  REGION 1..8: note = M2aM14178N HCDR2
ISDGGTYT

SEQ ID NO: 62 - protein, 12 aa, synthetic construct
  REGION 1..12: note = M2aM14178N HCDR3
IRGDVYYVRFAY

SEQ ID NO: 63 - protein, 109 aa, synthetic construct
  REGION 1..109: note = M2aM14178N LCVR
QAVVTQESALTTSPGETVTLTCRSSTGAVTSSNHANWVQEKPDHLFTGLIGGTNNRAPGVPARFSGSLIGDKAALTITGAQTEDEAIYFCALWHSNHWVFGGGTKLTVL

SEQ ID NO: 64 - protein, 9 aa, synthetic construct
  REGION 1..9: note = M2aM14178N LCDR1
TGAVTSSNH

SEQ ID NO: 65 - skipped

SEQ ID NO: 66 - protein, 9 aa, synthetic construct
  REGION 1..9: note = M2aM14178N LCDR3
ALWHSNHWV

SEQ ID NO: 67 - protein, 106 aa, synthetic construct
  REGION 1..106: note = M2aM14178N Constant LC
GQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 68 - protein, 119 aa, synthetic construct
  REGION 1..119: note = M2aM14179N HCVR
EVQLQQSGPELVKPGASVKISCKASGYTLTDYYMNWVKQSHGKSLEWIGDINPNNGGTSYNQKFKGKATLTVDKSSSTAYMELRSLTSEDSAVYYCARGAYYSNYFDYWGQGTTLTVSS

SEQ ID NO: 69 - protein, 8 aa, synthetic construct
  REGION 1..8: note = M2aM14179N HCDR1
GYTLTDYY

SEQ ID NO: 70 - protein, 8 aa, synthetic construct
  REGION 1..8: note = M2aM14179N HCDR2
INPNNGGT

SEQ ID NO: 71 - protein, 12 aa, synthetic construct
  REGION 1..12: note = M2aM14179N HCDR3
ARGAYYSNYFDY

SEQ ID NO: 72 - protein, 112 aa, synthetic construct
  REGION 1..112: note = M2aM14179N LCVR
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYILFTFGSGTQLEIK

SEQ ID NO: 73 - protein, 12 aa, synthetic construct
  REGION 1..12: note = M2aM14179N LCDR1
QSLLNSRTRKNY

SEQ ID NO: 74 - skipped

SEQ ID NO: 75 - protein, 8 aa, synthetic construct
  REGION 1..8: note = M2aM14179N LCDR3
KQSYILFT

SEQ ID NO: 76 - protein, 427 aa, synthetic construct
  REGION 1..427: note = TrkB(C32-H430).mmH aa 1-399: aa 32 through 430 of NP_001018074.1; aa 400-427 myc-myc-hexahistidine tag
CPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHEQKLISEEDLGGEQKLISEEDLHHHHHH

SEQ ID NO: 77 - protein, 632 aa, synthetic construct
  REGION 1..632: note = Human TrkB (C32-H430).mFc aa 1-399: aa 32 through 430 of NP_001018074.1; aa 400-632 mouse Fc domain
CPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 78 - protein, 626 aa, synthetic construct
  REGION 1..626: note = Human TrkB (C32-H430).hFc aa 1-399: aa 32 through 430 of NP_001018074.1; aa 400-626: human Fc domain
CPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 426 aa, synthetic construct
  REGION 1..426: note = Mouse TrkB (C32-H429)mmH aa 1-398: aa 32 through 429 of NP_001020245; aa 399-426: myc-myc-hexahistidine tag
CPTSCKCSSARIWCTEPSPGIVAFPRLEPNSVDPENITEILIANQKRLEIINEDDVEAYVGLRNLTIVDSGLKFVAYKAFLKNSNLRHINFTRNKLTSLSRRHFRHLDLSDLILTGNPFTCSCDIMWLKTLQETKSSPDTQDLYCLNESSKNMPLANLQIPNCGLPSARLAAPNLTVEEGKSVTLSCSVGGDPLPTLYWDVGNLVSKHMNETSHTQGSLRITNISSDDSGKQISCVAENLVGEDQDSVNLTVHFAPTITFLESPTSDHHWCIPFTVRGNPKPALQWFYNGAILNESKYICTKIHVTNHTEYHGCLQLDNPTHMNNGDYTLMAKNEYGKDERQISAHFMGRPGVDYETNPNYPEVLYEDWTTPTDIGDTTNKSNEIPSTDVADQSNREHEQKLISEEDLGGEQKLISEEDLHHHHHH

SEQ ID NO: 80 - protein, 631 aa, synthetic construct
  REGION 1..631: note = Mouse TrkB (C32-H429).mFc aa 1-398: aa 32 through 429 of NP_001020245; aa 399-631 Mouse Fc domain
CPTSCKCSSARIWCTEPSPGIVAFPRLEPNSVDPENITEILIANQKRLEIINEDDVEAYVGLRNLTIVDSGLKFVAYKAFLKNSNLRHINFTRNKLTSLSRRHFRHLDLSDLILTGNPFTCSCDIMWLKTLQETKSSPDTQDLYCLNESSKNMPLANLQIPNCGLPSARLAAPNLTVEEGKSVTLSCSVGGDPLPTLYWDVGNLVSKHMNETSHTQGSLRITNISSDDSGKQISCVAENLVGEDQDSVNLTVHFAPTITFLESPTSDHHWCIPFTVRGNPKPALQWFYNGAILNESKYICTKIHVTNHTEYHGCLQLDNPTHMNNGDYTLMAKNEYGKDERQISAHFMGRPGVDYETNPNYPEVLYEDWTTPTDIGDTTNKSNEIPSTDVADQSNREHEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 81 - protein, 625 aa, synthetic construct
  REGION 1..625: note = Mouse TrkB (C32-H429).hFc aa 1-398: aa 32 through 429 of NP_001020245; aa 399-625 human Fc domain
CPTSCKCSSARIWCTEPSPGIVAFPRLEPNSVDPENITEILIANQKRLEIINEDDVEAYVGLRNLTIVDSGLKFVAYKAFLKNSNLRHINFTRNKLTSLSRRHFRHLDLSDLILTGNPFTCSCDIMWLKTLQETKSSPDTQDLYCLNESSKNMPLANLQIPNCGLPSARLAAPNLTVEEGKSVTLSCSVGGDPLPTLYWDVGNLVSKHMNETSHTQGSLRITNISSDDSGKQISCVAENLVGEDQDSVNLTVHFAPTITFLESPTSDHHWCIPFTVRGNPKPALQWFYNGAILNESKYICTKIHVTNHTEYHGCLQLDNPTHMNNGDYTLMAKNEYGKDERQISAHFMGRPGVDYETNPNYPEVLYEDWTTPTDIGDTTNKSNEIPSTDVADQSNREHDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 427 aa, synthetic construct
  REGION 1..427: note = Rabbit TrkB (C32-H430)mmH aa 1-399: aa32 through 430 of XP_002721319.1; aa 400-427: myc-myc-hexahistidine tag
CPASCTCSASRIWCSDPVPGLMAFPRLEPNSADPENITEIFIANQKKLEIINEDDIEAYVGLRNLTIVDSGLKFVAYKAFLKNSNLQHINFTRNKLTSLSRKHFRHLDLSELILVGNPFTCSCDIMWIKTLQETKSSPETQDLYCLNESSKNIPLANLQIPNCGLPSASLAAPNLTVEEGKSITLSCNVAGDPVPNLYWDVGNLVSKHMNETSHTQGFLRITNISSDDSGKQISCVAENLVGEDQDSVNLTVHFAPTITFLESPTSDHHWCIPFTVKGNPKPTLQWFYNGAILNESKYICTKIHVTNHTEYHGCLQLDNPTHMNNGDYTLIAKNEYGKDERQISAHFMGWPGIDDDPNLNYPDVIYADYGTAANDIGDTTNRSNEIPPTGAADNAGREHEQKLISEEDLGGEQKLISEEDLHHHHHH

SEQ ID NO: 83 - protein, 632 aa, synthetic construct
  REGION 1..632: note = Rabbit TrkB (C32-H430)mFc aa 1-399: aa32 through 430 of XP_002721319.1; aa 400-632 Mouse Fc domain
CPASCTCSASRIWCSDPVPGLMAFPRLEPNSADPENITEIFIANQKKLEIINEDDIEAYVGLRNLTIVDSGLKFVAYKAFLKNSNLQHINFTRNKLTSLSRKHFRHLDLSELILVGNPFTCSCDIMWIKTLQETKSSPETQDLYCLNESSKNIPLANLQIPNCGLPSASLAAPNLTVEEGKSITLSCNVAGDPVPNLYWDVGNLVSKHMNETSHTQGFLRITNISSDDSGKQISCVAENLVGEDQDSVNLTVHFAPTITFLESPTSDHHWCIPFTVKGNPKPTLQWFYNGAILNESKYICTKIHVTNHTEYHGCLQLDNPTHMNNGDYTLIAKNEYGKDERQISAHFMGWPGIDDDPNLNYPDVIYADYGTAANDIGDTTNRSNEIPPTGAADNAGREHEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 84 - protein, 426 aa, synthetic construct
  REGION 1..426: note = Rat TrkB (C32-H429)mmH aa 1-398: aa 32 through 429 of NP_036863.1; aa 399-426 myc-myc-hexahistidine tag
CPMSCKCSTTRIWCTEPSPGIVAFPRLEPNSIDPENITEILIANQKRLEIINEDDVEAYVGLKNLTIVDSGLKFVAYKAFLKNGNLRHINFTRNKLTSLSRRHFRHLDLSDLILTGNPFTCSCDIMWLKTLQETKSSPDTQDLYCLNESSKNTPLANLQIPNCGLPSARLAAPNLTVEEGKSVTISCSVGGDPLPTLYWDVGNLVSKHMNETSHTQGSLRITNISSDDSGKQISCVAENLVGEDQDSVNLTVHFAPTITFLESPTSDHHWCIPFTVRGNPKPALQWFYNGAILNESKYICTKIHVTNHTEYHGCLQLDNPTHMNNGDYTLMAKNEYGKDERQISAHFMGRPGVDYETNPNYPEVLYEDWTTPTDIGDTTNKSNEIPSTDVADQTNREHEQKLISEEDLGGEQKLISEEDLHHHHHH

SEQ ID NO: 85 - protein, 631 aa, synthetic construct
  REGION 1..631: note = Rat TrkB (C32-H429)mFc aa 1-398: aa 32 through 429 of NP_036863.1; aa 399-631 Mouse Fc domain
CPMSCKCSTTRIWCTEPSPGIVAFPRLEPNSIDPENITEILIANQKRLEIINEDDVEAYVGLKNLTIVDSGLKFVAYKAFLKNGNLRHINFTRNKLTSLSRRHFRHLDLSDLILTGNPFTCSCDIMWLKTLQETKSSPDTQDLYCLNESSKNTPLANLQIPNCGLPSARLAAPNLTVEEGKSVTISCSVGGDPLPTLYWDVGNLVSKHMNETSHTQGSLRITNISSDDSGKQISCVAENLVGEDQDSVNLTVHFAPTITFLESPTSDHHWCIPFTVRGNPKPALQWFYNGAILNESKYICTKIHVTNHTEYHGCLQLDNPTHMNNGDYTLMAKNEYGKDERQISAHFMGRPGVDYETNPNYPEVLYEDWTTPTDIGDTTNKSNEIPSTDVADQTNREHEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 86 - protein, 605 aa, synthetic construct
  REGION 1..605: note = Human TrkA(A34-T414).hFc aa 1-375: aa 34 through 414 of NP_001012331.1 with V263L, C300S; aa 376-378 GPG Linker; aa 379-605 human Fc domain
APCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNLTCWAENDVGRAEVSVQVNVSFPASVQLHTAVEMHHWSIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETGPGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 426 aa, synthetic construct
  REGION 1..426: note = Human TrkC(C32-T429).mmH aa 1-398: aa 32 through 429 of NP_001012338.1; aa 399-426 myc-myc-hexahistidine tag
CPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTEQKLISEEDLGGEQKLISEEDLHHHHHH

SEQ ID NO: 88 - protein, 426 aa, synthetic construct
  REGION 1..426: note = Mouse TrkC(C32-T429).mmH aa 1-398: aa 32 through 429 of NP_032772.3; aa 399-426 myc-myc-hexahistidine tag
CPANCVCSKTEINCRRPDDGNLFPLLEGQDSGNSNGNASINITDISRNITSIHIENWRGLHTLNAVDMELYTGLQKLTIKNSGLRNIQPRAFAKNPHLRYINLSSNRLTTLSWQLFQTLSLRELRLEQNFFNCSCDIRWMQLWQEQGEARLDSQSLYCISADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLVEPEVRLEHCIEFVVRGNPTPTLHWLYNGQPLRESKIIHMDYYQEGEVSEGCLLFNKPTHYNNGNYTLIAKNALGTANQTINGHFLKEPFPESTDFFDFESDASPTPPITVTHKPEEDTEQKLISEEDLGGEQKLISEEDLHHHHHH

SEQ ID NO: 89 - protein, 426 aa, synthetic construct
  REGION 1..426: note = MF TrkC(C32-T429).mmH aa 1-398: aa 32 through 429 of XP_015308837.1; aa 399-426 myc-myc-hexahistidine tag
CPANCVCSKTEINCRRPDDGNLFPLLEGQDSGNSNGNASINITDISRNITSIHIENWRSLHTLNAVDMELYTGLQNLTIRNSGLRSIQPRAFAKNPHLRYINLSSNRLTTLSWQLFQTLSLRELRLEQNFFNCSCDIRWMQLWQEQGEAKLNNQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTEQKLISEEDLGGEQKLISEEDLHHHHHH

SEQ ID NO: 90 - protein, 120 aa, synthetic construct
  REGION 1..120: note = BDNF_isoform A aa 1-120: aa 129 through 247 of NP_733928.1 with a Met added on the N-term
MHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 91 - protein, 791 aa, synthetic construct
  REGION 1..791: note = TrkB (pRG984.hTrkB ecto-hTrkB TM/Cyto) aa 1-792: aa 32 through 822 of NP_001018074.1 or Uniprot number Q16620-1 TM/Cyto 399-790
CPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 92 - protein, 445 aa, synthetic construct
  REGION 1..445: note = Mouse TrkB aa 1-446: aa 32 through 476 of NP_032771.1
CPTSCKCSSARIWCTEPSPGIVAFPRLEPNSVDPENITEILIANQKRLEIINEDDVEAYVGLRNLTIVDSGLKFVAYKAFLKNSNLRHINFTRNKLTSLSRRHFRHLDLSDLILTGNPFTCSCDIMWLKTLQETKSSPDTQDLYCLNESSKNMPLANLQIPNCGLPSARLAAPNLTVEEGKSVTLSCSVGGDPLPTLYWDVGNLVSKHMNETSHTQGSLRITNISSDDSGKQISCVAENLVGEDQDSVNLTVHFAPTITFLESPTSDHHWCIPFTVRGNPKPALQWFYNGAILNESKYICTKIHVTNHTEYHGCLQLDNPTHMNNGDYTLMAKNEYGKDERQISAHFMGRPGVDYETNPNYPEVLYEDWTTPTDIGDTTNKSNEIPSTDVADQSNREHLSVYAVVVIASVVGFCLLVMLLLLKLARHSKFGMKGFVLFHKIPLDG

SEQ ID NO: 93 - protein, 790 aa, synthetic construct
  REGION 1..790: note = Chimera TrkB (pRG984.mTrkB ecto-hTrkB TM/Cyto) aa 1-398: aa 32 through 429 of NP_001020245.1 or Uniprot number P15209-1; Human TrkB TM/Cyto 399-790 (aaa 431 through 822 of NP_001018074.1)
CPTSCKCSSARIWCTEPSPGIVAFPRLEPNSVDPENITEILIANQKRLEIINEDDVEAYVGLRNLTIVDSGLKFVAYKAFLKNSNLRHINFTRNKLTSLSRRHFRHLDLSDLILTGNPFTCSCDIMWLKTLQETKSSPDTQDLYCLNESSKNMPLANLQIPNCGLPSARLAAPNLTVEEGKSVTLSCSVGGDPLPTLYWDVGNLVSKHMNETSHTQGSLRITNISSDDSGKQISCVAENLVGEDQDSVNLTVHFAPTITFLESPTSDHHWCIPFTVRGNPKPALQWFYNGAILNESKYICTKIHVTNHTEYHGCLQLDNPTHMNNGDYTLMAKNEYGKDERQISAHFMGRPGVDYETNPNYPEVLYEDWTTPTDIGDTTNKSNEIPSTDVADQSNREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 94 - protein, 791 aa, synthetic construct
  REGION 1..791: note = AGM TrkB (pRGN984.agmTrkB) 1-791: aa 32 through 822 of XP_007967815.1
CPRSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 95 - protein, 807 aa, synthetic construct
  REGION 1..807: note = MM TrkB (mmTrkB) 1-807: aa 32 through 838 of NP_001248226.1
CPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKDFSWFGFGKVKSRQGVGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 96 - protein, 807 aa, synthetic construct; identical to SEQ ID NO: 95
  REGION 1..807: note = MF TrkB (mfTrkB) 1-807 : aa 32 through 838 of XP_005582102.1

SEQ ID NO: 97 - protein, 447 aa, synthetic construct
  REGION 1..447: note = H1M8037C heavy chain
QVQLVQSGAEVKKPGASVKVSCKASGYTFTNYDIIWVRQAPGQGLEWMGYINPYNDGTKYNEKFKGRVTMTRDTSTSTVYMELSSLRSEDTAVYYCARLLKYRRFRYYAIDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 98 - protein, 214 aa, synthetic construct
  REGION 1..214: note = H1M8037C light chain
DIQMTQSPSSLSASVGDRVTITCRTSENVYSNLAWYQQKPGKAPKLLIYAASNLQSGVPSRFSGSGSGTDYTFTISSLQPEDIATYYCQHFWGSPFTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 99 - protein, 445 aa, synthetic construct
  REGION 1..445: note = H4H9780P Full length heavy chain
QVQLVESGGGVVQPGRSLRLSCAASGFSFSSFGMHWVRQAPGKGLEWVSVISYDGINTYYTDSVKGRFTISRDNSKNTLYLQMNGLRAEDTALYYCVQGSIGTVFEYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 214 aa, synthetic construct
  REGION 1..214: note = H4H9780P Full length light chain
DIQMTQSPSSLSASVGDRVTITCRASQGISNYLAWYQQKPGKVPKLLIYAASTLQSGVPSRFGGSGSGTDFTLTISSLQPEDVATYYCQKYTSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 449 aa, synthetic construct
  REGION 1..449: note = H4H9814P Full length heavy chain
EVQLVESGGGLVQPGGSLKLSCTASGFTFSGSVIHWVRQASGKGLEWIGRIRNKANSYATAYGASVTGRFTISRDDSKNTAYLQMNSLKTEDTAVYYCTFPGVVGRGGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 220 aa, synthetic construct
  REGION 1..220: note = H4H9814P Full length light chain
DIVMTQSPDSLAVSLGEGATINCMSSQSVLFSSNNKNYLAWYQQKPGQPPKLLFYWASTRESGVPDRFGGSGSGTDFSLTINSLQTEDVAVYYCLQYYSIPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 454 aa, synthetic construct
  REGION 1..454: note = H4H9816P2 Full length heavy chain
EVQLVESGGGLVQPGGSLTLSCAASGFNFRDYEMIWVRQTPGKGLEWISYISNSGYTIYYADSVKGRFTISRDNARNSIYLQVNSLRAEDTAVYYCSRRTTMIRGIRAYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 104 - protein, 215 aa, synthetic construct
  REGION 1..215: note = H4H9816P2 Full length light chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0309100 A1
(43) Pub. Date: Sep. 19, 2024

(54) Anti-Trkb Monoclonal Antibodies And Methods Of Use

(71) Applicant: Regeneron Pharmaceuticals, Inc., Tarrytown, NY (US)

(72) Inventors: Susan D. Croll, Putnam Valley, NY (US); Min Gao, Montvale, NJ (US); Ying Hu, Scarsdale, NY (US)

(21) Appl. No.: 18/737,782

(22) Filed: Jun. 7, 2024

Related U.S. Application Data
(63) Continuation of application No. 17/342,749, filed on Jun. 9, 2021, now Pat. No. 12,037,401.
Application No. 17/342,749 is a division of application No. 16/202,881, filed on Nov. 28, 2018, now Pat. No. 11,066,474.
(60) Provisional application No. 62/592,657, filed on Nov. 30, 2017.

(51) Int. Cl. C07K 16/28; A61K 9/00; A61K 39/00; A61P 25/00

(52) U.S. Cl. CPC C07K 16/2863; A61K 9/0019; A61P 25/00; A61K 2039/505; C07K 2317/21; C07K 2317/56; C07K 2317/565; C07K 2317/75; C07K 2317/76; C07K 2317/92; C07K 2317/94

(57) ABSTRACT

The present invention provides antibodies that bind specifically to TrkB and methods of using the same. According to certain embodiments, the antibodies of the invention are agonist antibodies that are neuroprotective, as shown by their effect on enhancing the survival of retinal ganglion cells in vitro. As such, these agonist antibodies may be used to treat diseases or disorders of the eye, such as, but not limited to glaucoma. In addition, other neuronal diseases or disorders may benefit from treatment with these agonist antibodies, including any disease or disorder characterized in part by neuronal damage. In certain embodiments, the invention includes antibodies that bind TrkB and mediate cell signaling. The antibodies of the invention may be fully human, non-naturally occurring antibodies.

### CROSS-REFERENCE

This application is a continuation of U.S. application Ser. No. 17/342,749, filed on Jun. 9, 2021, which is a divisional of U.S. application Ser. No. 16/202,881, filed on Nov. 28, 2018, which claims the benefit of U.S. provisional patent application Ser. No. 62/592,657 filed Nov. 30, 2017 which is incorporated by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML, REGN-029CON, created on Jun. 7, 2024 and having a size of 141,020 bytes. The contents of the Sequence Listing XML are incorporated herein by reference in their entirety.

### FIELD OF THE INVENTION

The present invention relates to anti-tropomyosin-receptor-kinase B (TrkB) monoclonal antibodies. More specifically, the invention relates to compositions comprising anti-TrkB monoclonal antibodies and methods of using these antibodies.

### BACKGROUND

Tropomyosin receptor kinase B (TrkB), belongs to a family of single transmembrane receptor tyrosine kinases, which includes TrkA and TrkC. These receptor kinases mediate the activity of neurotrophins, which are required for neuronal survival and development. Neurotrophins include, but are not limited to, nerve growth factor (NGF), brain derived neurotrophic factor (BDNF), neurotrophin-3 (NT-3), and neurotrophin-4/5 (NT-4/5). (Lo, K Y et al., J. Biol. Chem., 280:41744-52 (2005)).

TrkB is the high affinity receptor for BDNF (Minichiello, et al., Neuron 21:335-45 (1998)), but it is also known to bind NT4/5. The binding of BDNF to trkB causes the receptor to dimerize, resulting in the autophosphorylation of specific tyrosine residues on the receptor and activation of signaling pathways involving mitogen-activated protein kinase (MAPK), phosphatidylinositol 3-kinase (PI3K), and phospholipase C-γ(PLC-γ). (Jing, et al. Neuron 9:1067-1079 (1992); Barbacid, J. Neurobiol. 25:1386-1403 (1994); Bothwell, Ann. Rev. Neurosci. 18:223 253 (1995); Segal and Greenberg, Ann. Rev. Neurosci. 19:463 489 (1996); Kaplan and Miller, Curr. Opinion Neurobiol. 10:381 391 (2000)). After binding to BDNF, TrkB mediates the multiple effects of the neurotrophin, which includes neuronal differentiation and survival.

Since TrkB plays a major role in neuronal survival, differentiation, and function, TrkB agonists may have therapeutic potential for treating a number of neurodegenerative and metabolic disorders.

Certain TrkB agonists have been described in US2010/0150914; US2003/0157099; US2010/0196390 and US2017/0157099. However, there is still a need for the identification and development of additional TrkB agonists that provide improved specificity in addition to exhibiting neuronal survival and neuroprotective properties, such as those described herein.

### BRIEF SUMMARY OF THE INVENTION

The present invention provides isolated monoclonal antibodies and antigen-binding fragments thereof that specifically bind to tropomyosin-receptor-kinase B (TrkB). The isolated antibodies and antigen-binding fragments of the invention are useful for treating diseases and disorders associated with TrkB activity or expression.

In its broadest aspect, the invention provides anti-TrkB agonist antibodies, which activate TrkB and promote neuronal survival. These antibodies may be used to improve nerve function and for treating any disease or disorder characterized in part by cellular degeneration, including nerve cell damage associated with nervous system injury and/or chronic neurodegenerative diseases.

In certain embodiments, the anti-TrkB antibodies may be useful to treat various diseases or disorders of the eye and may be formulated for intraocular or intravitreal delivery, in order to treat diseases of the eye, such as, but not limited to, glaucoma.

The antibodies of the invention can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., 2000, J. Immunol. 164: 1925-1933).

Exemplary anti-TrkB antibodies of the present invention are listed in Tables 1 and 2 herein. Table 1 sets forth the amino acid sequence identifiers of the heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the exemplary anti-TrkB antibodies. Table 2 sets forth the nucleic acid sequence identifiers of the HCVRs, LCVRs, HCDR1, HCDR2 HCDR3, LCDR1, LCDR2 and LCDR3 of the exemplary anti-TrkB antibodies.

The present invention provides antibodies or antigen-binding fragments thereof that specifically bind TrkB, comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

The present invention also provides antibodies or antigen-binding fragments thereof that specifically bind TrkB, comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

The present invention also provides antibodies or antigen-binding fragments thereof that specifically bind TrkB, comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present invention provides antibodies, or antigen-binding fragments thereof, comprising an HCVR/LCVR amino acid sequence pair contained within any of the exemplary anti-TrkB antibodies listed in Table 1.

Accordingly, in a first aspect, the invention provides an isolated antibody or antigen-binding fragment thereof that binds specifically to tropomyosin receptor kinase B (TrkB), wherein the antibody or antigen-binding fragment thereof comprises three heavy chain complementarity determining regions (CDRs) (HCDR1, HCDR2 and HCDR3) contained within a heavy chain variable region (HCVR) comprising an amino acid sequence as set forth in Table 1, or a substantially similar sequence thereof having at least 90% sequence identity thereto; and three light chain CDRs (LCDR1, LCDR2 and LCDR3) contained within a light chain variable region (LCVR) comprising an amino acid sequence as set forth in Table 1, or a substantially similar sequence thereof having at least 90% sequence identity thereto.

In one embodiment, the anti-TrkB antibody or antigen-binding fragment thereof exhibits one or more properties selected from the group consisting of:
(a) is an agonist antibody;
(b) binds human TrkB with a K_{D} of less than about 200 nM as measured by surface plasmon resonance at 25° C. or at 37° C.;
(c) binds human TrkB with a dissociative half life (t½) of greater than about 10 minutes as measured by surface plasmon resonance at 25° C. or at 37° C.;
(d) activates human TrkB signaling in the absence of brain derived neurotrophic factor (BDNF) in cells engineered to express human TrkB with an EC₅₀ ranging from about 35 to 82 pM;
(e) enhances TrkB phosphorylation when injected into the hippocampus of mice homozygous for human TrkB receptor (TrkB^{hu/hu});
(f) promotes weight loss when injected into mice homozygous for human TrkB receptor (TrkB^{hu/hu});
(g) increases retinal ganglion cell (RGC) survival as assessed in an optic nerve transection model in humanized TrkB rats;
(h) activates the MAPK/ERK and PI3K/Akt signaling pathways;
(i) increases survival of neuronal cells in vitro; and
(j) blocks the binding of TrkB to BDNF and/or NT-4 with an IC₅₀ of less than 5 nM.

In one embodiment, the invention provides an antibody or antigen-binding fragment thereof that specifically binds tropomyosin-receptor-kinase B (TrkB), wherein the antibody or antigen-binding fragment thereof comprises: (a) the complementarity determining regions (CDRs) of a heavy chain variable region (HCVR) comprising an amino acid sequence as set forth in Table 1; and (b) the CDRs of a light chain variable region (LCVR) comprising an amino acid sequence as set forth in Table 1.

In one embodiment, the antibody or antigen-binding fragment thereof that specifically binds TrkB comprises three heavy chain CDRs (HCDR1, HCDR2 and HCDR3) contained within any one of the HCVR sequences selected from the group consisting of SEQ ID NOs: 2, 18, 34, 49, 59 and 68, or a substantially similar sequence thereof having at least 90% sequence identity thereto; and three light chain CDRs (LCDR1, LCDR2 and LCDR3) contained within any one of the LCVR sequences selected from the group consisting of SEQ ID NOs: 10, 26, 42, 53, 63 and 72, or a substantially similar sequence thereof having at least 90% sequence identity thereto.

In one embodiment, the isolated antibody or antigen-binding fragment thereof that specifically binds TrkB comprises a HCVR having an amino acid sequence selected from the group consisting of SEQ ID NOs: 2, 18, 34, 49, 59 and 68.

In one embodiment, the isolated antibody or antigen-binding fragment thereof that specifically binds TrkB further comprises a LCVR having an amino acid sequence selected from the group consisting of SEQ ID NOs: 10, 26, 42, 53, 63 and 72.

In one embodiment, the isolated antibody or antigen-binding fragment thereof that specifically binds TrkB comprises a HCVR having an amino acid sequence selected from the group consisting of SEQ ID NOs: 2, 18, 34, 49, 59 and 68; and a LCVR having an amino acid sequence selected from the group consisting of SEQ ID NOs: 10, 26, 42, 53, 63 and 72.

In one embodiment, the isolated antibody or antigen-binding fragment thereof that specifically binds TrkB comprises the CDRs of a HCVR/LCVR amino acid sequence pair selected from the group consisting of SEQ ID NOs: 2/10, 18/26, 34/42, 49/53, 59/63 and 68/72.

In one embodiment, the isolated antibody or antigen-binding fragment thereof that specifically binds TrkB comprises: the CDRs of a HCVR/LCVR amino acid sequence pair selected from the group consisting of SEQ ID NOs: 2/10, 18/26, and 34/42.

In one embodiment, the isolated antibody or antigen-binding fragment thereof that specifically binds TrkB comprises an HCVR/LCVR amino acid sequence pair selected from the group consisting of SEQ ID NOs: 2/10, 18/26, 34/42, 49/53, 59/63 and 68/72.

In one embodiment, the isolated antibody or antigen-binding fragment thereof that specifically binds TrkB comprises an HCVR/LCVR amino acid sequence pair selected from the group consisting of SEQ ID NOs: 2/10, 18/26 and 34/42.

The present invention also provides antibodies or antigen-binding fragments thereof that specifically bind TrkB, comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity thereto.

The present invention also provides antibodies or antigen-binding fragments thereof that specifically bind TrkB, comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity thereto.

The present invention also provides antibodies or antigen-binding fragments thereof that specifically bind TrkB, comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity thereto.

The present invention also provides antibodies or antigen-binding fragments thereof that specifically bind TrkB, comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity thereto.

The present invention also provides antibodies or antigen-binding fragments thereof that specifically bind TrkB, comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity thereto.

The present invention also provides antibodies or antigen-binding fragments thereof that specifically bind TrkB, comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity thereto.

The present invention also provides antibodies or antigen-binding fragments thereof that specifically bind TrkB, comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, the present invention provides antibodies, or antigen-binding fragments thereof, comprising an HCDR3/LCDR3 amino acid sequence pair contained within any of the exemplary anti-TrkB antibodies listed in Table 1. In certain embodiments, the HCDR3/LCDR3 amino acid sequence pair is selected from the group consisting of: 8/16, 24/32, 40/48, 52/56, 62/66 and 71/75.

The present invention also provides antibodies or antigen-binding fragments thereof that specifically bind TrkB, comprising a set of six CDRs (i.e., HCDR1, HCDR2, HCDR3, LCDR1, LCDR2, LCDR3) contained within any of the exemplary anti-TrkB antibodies listed in Table 1. In certain embodiments, the HCDR1, HCDR2, HCDR3, LCDR1, LCDR2, LCDR3 amino acid sequence set is selected from the group consisting of: (a) SEQ ID NOs: 4, 6, 8, 12, 14, 16; (b) SEQ ID NOs: 20, 22, 24, 28, 30, 32; (c) SEQ ID NOs: 36, 38, 40, 44, 46, 48; (d) SEQ ID NOs: 50, 51, 52, 54, 55, 56; (c) SEQ ID NOs: 60, 61, 62, 64, 65, 66; and (f) SEQ ID NOs: 69, 70, 71, 73, 74, 75.

In a related embodiment, the present invention provides antibodies, or antigen-binding fragments thereof that specifically bind TrkB, comprising a set of six CDRs (i.e., HCDR1, HCDR2, HCDR3, LCDR1, LCDR2, LCDR3) contained within an HCVR/LCVR amino acid sequence pair as defined by any of the exemplary anti-TrkB antibodies listed in Table 1. For example, the present invention includes antibodies or antigen-binding fragments thereof that specifically bind TrkB, comprising the HCDR1, HCDR2, HCDR3, LCDR1, LCDR2, LCDR3 amino acid sequences set contained within an HCVR/LCVR amino acid sequence pair selected from the group consisting of: 2/10, 18/26, 34/42, 49/53, 59/63, and 68/72. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In one embodiment, the invention provides an isolated antibody or antigen-binding fragment thereof that specifically binds TrkB comprising:
(a) a HCDR1 domain having an amino acid sequence selected from the group consisting of SEQ ID NOs: 4, 20, 36, 50, 60 and 69;
(b) a HCDR2 domain having an amino acid sequence selected from the group consisting of SEQ ID NOs: 6, 22, 38, 51, 61 and 70;
(c) a HCDR3 domain having an amino acid sequence selected from the group consisting of SEQ ID NOs: 8, 24, 40, 52, 62 and 71;
(d) a LCDR1 domain having an amino acid sequence selected from the group consisting of SEQ ID NOs: 12, 28, 44, 54, 64 and 73;
(c) a LCDR2 domain having an amino acid sequence selected from the group consisting of SEQ ID NOs: 14, 30, 46, 55, 65 and 74; and
(f) a LCDR3 domain having an amino acid sequence selected from the group consisting of SEQ ID NOs: 16, 32, 48, 56, 66 and 75.

In one embodiment, the invention provides an isolated antibody or antigen-binding fragment thereof that specifically binds TrkB comprising:
(a) a HCDR1 domain having an amino acid sequence selected from the group consisting of SEQ ID NOs: 4, 20 and 36;
(b) a HCDR2 domain having an amino acid sequence selected from the group consisting of SEQ ID NOs: 6, 22 and 38;
(c) a HCDR3 domain having an amino acid sequence selected from the group consisting of SEQ ID NOs: 8, 24 and 40;
(d) a LCDR1 domain having an amino acid sequence selected from the group consisting of SEQ ID NOs: 12, 28 and 44;
(e) a LCDR2 domain having an amino acid sequence selected from the group consisting of SEQ ID NOs: 14, 30 and 46; and
(f) a LCDR3 domain having an amino acid sequence selected from the group consisting of SEQ ID NOs: 16, 32 and 48.

In one embodiment, the isolated antibody or antigen-binding fragment thereof comprises a set of six CDRs (HCDR1-HCDR2-HCDR3-LCDR1-LCDR2-LCDR3) selected from the group consisting of: (a) SEQ ID NOs: 4-6-8-12-14-16; (b) SEQ ID NOs: 20-22-24-28-30-32; (c) SEQ ID NOs: 36-38-40-44-46-48; (d) SEQ ID NOs: 50-51-52-54-55-56; (e) SEQ ID NOs: 60-61-62-64-65-66; and (f) SEQ ID NOs: 69-70-71-73-74-75.

In one embodiment, the isolated antibody or antigen-binding fragment thereof that binds to TrkB comprises an antibody or antigen-binding fragment thereof that competes for binding to TrkB with a reference antibody, wherein the reference antibody comprises an HCVR/LCVR amino acid sequence pair selected from the group consisting of SEQ ID NOs: 2/10, 18/26, 34/42, 49/53, 59/63 and 68/72.

In one embodiment, the isolated antibody or antigen-binding fragment thereof that binds to TrkB comprises an antibody or antigen-binding fragment thereof that binds to the same epitope as a reference antibody, wherein the reference antibody comprises an HCVR/LCVR amino acid sequence pair selected from the group consisting of SEQ ID NOs: 2/10, 18/26, 34/42, 49/53, 59/63 and 68/72.

In one embodiment, the isolated antibody or antigen-binding fragment thereof binds human TrkB with a K_{D} of less than about 300 nM as measured by surface plasmon resonance at 25° C. or 37° C.

In one embodiment, the isolated antibody or antigen-binding fragment thereof binds human TrkB with a K_{D} of less than about 200 nM as measured by surface plasmon resonance at 25° C. or 37° C.

In one embodiment, the isolated antibody or antigen-binding fragment thereof binds human TrkB with a K_{D} of less than about 150 nM as measured by surface plasmon resonance at 25° C. or 37° C.

In one embodiment, the isolated antibody or antigen-binding fragment thereof binds human TrkB with a K_{D} of less than about 50 nM as measured by surface plasmon resonance at 25° C. or 37° C.

In one embodiment, the isolated antibody or antigen-binding fragment thereof binds human TrkB with a K_{D} of less than about 100 pM as measured by surface plasmon resonance at 25° C. or 37° C.

In one embodiment, the isolated antibody or antigen-binding fragment thereof binds human TrkB with a dissociative half life (t½) of greater than about 10 minutes as measured by surface plasmon resonance at 25° C. or 37° C.

In one embodiment, the isolated antibody or antigen-binding fragment thereof binds human TrkB with a t½ of greater than about 40 minutes as measured by surface plasmon resonance at 25° C. or 37° C.

In one embodiment, the isolated antibody or antigen-binding fragment thereof binds human TrkB with a t½ of greater than about 120 minutes as measured by surface plasmon resonance at 25° C. or 37° C.

In one embodiment, the isolated antibody or antigen-binding fragment thereof that binds to TrkB activates human TrkB signaling in the absence of BDNF in cells engineered to express TrkB, with an EC₅₀ of less than about 100 pM.

In one embodiment, the isolated antibody or antigen-binding fragment thereof that binds to TrkB activates human TrkB signaling in the absence of BDNF in cells engineered to express TrkB, with an EC₅₀ ranging from about 35 pM to about 82 pM.

In one embodiment, the isolated antibody or antigen-binding fragment thereof that binds to TrkB enhances activation of human TrkB signaling in the presence of BDNF in cells engineered to express TrkB, with an EC₅₀ of less than about 100 pM.

In one embodiment, the isolated antibody or antigen-binding fragment thereof that binds to TrkB, which when injected into the hippocampus of humanized TrkB mice, demonstrates TrkB activation, as shown by an increase in TrkB phosphorylation.

In one embodiment, the isolated antibody or antigen-binding fragment thereof that binds to TrkB demonstrates activation of the MAPK/ERK and PI3K/Akt signaling pathways, as demonstrated following incubation of primary mouse cortical neurons with an agonist anti-TrkB antibody.

In one embodiment, the isolated antibody or antigen-binding fragment thereof that binds to TrkB enhances/increases survival of retinal ganglion cells as shown in an optic nerve transection model in TrkB humanized rats.

In one embodiment, the isolated antibody or antigen-binding fragment thereof that binds to TrkB enhances/increases survival of neuronal cells in vitro.

In one embodiment, the isolated antibody or antigen-binding fragment thereof that binds to TrkB promotes weight loss in humanized TrkB mice.

In one embodiment, the isolated antibody or antigen-binding fragment thereof that binds to TrkB promotes a loss of fat mass in humanized TrkB mice.

In one embodiment, the isolated antibody or antigen-binding fragment thereof that binds to TrkB promotes a decrease in food and water consumption in humanized TrkB mice.

In one embodiment, the isolated antibody or antigen-binding fragment thereof that binds to TrkB promotes an increase in locomotor activity in humanized TrkB mice.

In one embodiment, the invention provides an anti-TrkB antibody or antigen-binding fragment thereof that specifically binds TrkB and blocks TrkB binding to BDNF with an IC₅₀ of less than about 5 nM.

In one embodiment, the invention provides an anti-TrkB antibody or antigen-binding fragment thereof that specifically binds TrkB and blocks TrkB binding to BDNF with an IC₅₀ of less than about 500 pM.

In one embodiment, the invention provides an anti-TrkB antibody or antigen-binding fragment thereof that specifically binds TrkB and blocks TrkB binding to BDNF with an IC₅₀ of less than about 200 pM.

In a second aspect, the present invention provides nucleic acid molecules encoding anti-TrkB antibodies or portions thereof. For example, the present invention provides nucleic acid molecules encoding any of the HCVR amino acid sequences listed in Table 1; in certain embodiments the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto.

The present invention also provides nucleic acid molecules encoding any of the LCVR amino acid sequences listed in Table 1; in certain embodiments the nucleic acid molecule comprises a polynucleotide sequence selected from any of the LCVR nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR1 amino acid sequences listed in Table 1; in certain embodiments the nucleic acid molecule comprises a polynucleotide sequence selected from any of the HCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR2 amino acid sequences listed in Table 1; in certain embodiments the nucleic acid molecule comprises a polynucleotide sequence selected from any of the HCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR3 amino acid sequences listed in Table 1; in certain embodiments the nucleic acid molecule comprises a polynucleotide sequence selected from any of the HCDR3 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR1 amino acid sequences listed in Table 1; in certain embodiments the nucleic acid molecule comprises a polynucleotide sequence selected from any of the LCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR2 amino acid sequences listed in Table 1; in certain embodiments the nucleic acid molecule comprises a polynucleotide sequence selected from any of the LCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR3 amino acid sequences listed in Table 1; in certain embodiments the nucleic acid molecule comprises a polynucleotide sequence selected from any of the LCDR3 nucleic acid sequences listed in Table 2, or a substantially similar sequence thereof having at least 90%, at least 95%, at least 98% or at least 99% sequence identity thereto.

The present invention also provides nucleic acid molecules encoding an HCVR, wherein the HCVR comprises a set of three CDRs (i.e., HCDR1, HCDR2, HCDR3), wherein the HCDR1, HCDR2, HCDR3 amino acid sequence set is as defined by any of the exemplary anti-TRKB antibodies listed in Table 1.

The present invention also provides nucleic acid molecules encoding an LCVR, wherein the LCVR comprises a set of three CDRs (i.e., LCDR1, LCDR2, LCDR3), wherein the LCDR1, LCDR2, LCDR3 amino acid sequence set is as defined by any of the exemplary anti-TRKB antibodies listed in Table 1.

The present invention also provides nucleic acid molecules encoding both an HCVR and an LCVR, wherein the HCVR comprises an amino acid sequence of any of the HCVR amino acid sequences listed in Table 1, and wherein the LCVR comprises an amino acid sequence of any of the LCVR amino acid sequences listed in Table 1. In certain embodiments, the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto, and a polynucleotide sequence selected from any of the LCVR nucleic acid sequences listed in Table 2, or a substantially similar sequence thereof having at least 90%, at least 95%, at least 98% or at least 99% sequence identity thereto. In certain embodiments according to this aspect of the invention, the nucleic acid molecule encodes an HCVR and LCVR, wherein the HCVR and LCVR are both derived from the same anti-TrkB antibody listed in Table 1.

In a third aspect, the present invention provides recombinant expression vectors capable of expressing a polypeptide comprising a heavy or light chain variable region of an anti-TrkB antibody. For example, the present invention includes recombinant expression vectors comprising any of the nucleic acid molecules mentioned above, i.e., nucleic acid molecules encoding any of the HCVR, LCVR, and/or CDR sequences as set forth in Table 1. Also included within the scope of the present invention are host cells into which such vectors have been introduced, as well as methods of producing the antibodies or portions thereof by culturing the host cells under conditions permitting production of the antibodies or antibody fragments, and recovering the antibodies and antibody fragments so produced.

The present invention includes anti-TrkB antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

In a fourth aspect, the invention provides a pharmaceutical composition comprising at least one antibody of the invention, or an antigen binding fragment thereof, which specifically binds TrkB and a pharmaceutically acceptable carrier.

In a related aspect, the invention features a composition, which is a combination of an anti-TrkB antibody and a second therapeutic agent. In one embodiment, the second therapeutic agent is any agent that is advantageously combined with an anti-TrkB antibody. The second therapeutic agent may be useful for alleviating at least one symptom of the neurodegenerative disease or disorder.

In a fifth aspect, the invention provides a method for enhancing a biological activity mediated by TrkB, the method comprising contacting TrkB with a biologically effective amount of an agonist anti-TrkB antibody of Table 1, or contacting TrkB with a pharmaceutical composition containing a biologically effective amount of an agonist anti-TrkB antibody of Table 1.

In certain embodiments, the biological activity is neuronal protection or neuronal survival and neuronal protection or neuronal survival is enhanced upon contact of TrkB with an agonist anti-TrkB antibody.

In certain embodiments, the biological activity is neuroprotection and survival of retinal ganglion cells (RGCs).

In a sixth aspect, the invention provides therapeutic methods for treating a disease or disorder associated with TrkB activity or expression, or at least one symptom associated with the disease or disorder, using an anti-TrkB antibody or antigen-binding portion of an antibody of the invention. The therapeutic methods according to this aspect of the invention comprise administering a therapeutically effective amount of a pharmaceutical composition comprising an antibody or antigen-binding fragment of an antibody of the invention to a subject in need thereof. The disorder treated is any disease or condition which is improved, ameliorated, inhibited or prevented by targeting TrkB and/or by activating TrkB-mediated cell signaling.

In one embodiment, the anti-TrkB antibodies of the invention may provide a method of preventing injury or death of retinal neurons. In one embodiment, the anti-TrkB antibodies of the invention may provide a method of treating pathological diseases wherein degeneration of the retina occurs. In one embodiment, the anti-TrkB antibodies of the invention may provide a method of treating the living eye prior to or following ocular surgery, exposure to light or other environmental trauma thereby preventing degeneration of retinal cells. In one embodiment, the anti-TrkB antibodies of the invention may provide a method of preventing photoreceptor injury and degeneration in the living eye. In one embodiment, the anti-TrkB antibodies of the invention may provide a method of protecting retinal neurons without the induction of side effects, possibly due to cross reactivity with other receptors, such as the p75 receptor. In one embodiment, the anti-TrkB antibodies of the invention may provide a method of allowing injured photoreceptors to recover or regenerate.

In certain embodiments, the disease or disorder to be treated with an antibody of the invention is a disease or disorder of the eye selected from the group consisting of glaucoma, diabetic retinopathy, age-related macular degeneration, ischemic optic neuropathy, optic neuritis, retinal ischemia, photoreceptor degeneration, retinitis pigmentosa, Leber Congenital Amaurosis, Leber's hereditary optic neuropathy, Usher Syndrome, Stargardt disease and retinal artery or vein occlusions.

Other pathological conditions treatable with one or more anti-TrkB antibodies of the invention include retinal detachment, photic retinopathies, surgery-induced retinopathies (either mechanically or light-induced), toxic retinopathies, retinopathy of prematurity, viral retinopathies such as CMV or HIV retinopathy related to AIDS; uveitis; ischemic retinopathies due to venous or arterial occlusion or other vascular disorder, retinopathies due to trauma or penetrating lesions of the eye, peripheral vitreoretinopathy or inherited retinal degenerations.

In one embodiment, the disease or disorder of the eye to be treated with an agonist anti-TrkB antibody of the invention is glaucoma.

A seventh aspect of the invention provides for achieving a reduction in body weight in a subject, the method comprising administering a TrkB agonist antibody of Table 1, or a pharmaceutical composition comprising the antibody or antigen-binding fragment thereof, to the subject.

In a related aspect, the invention provides a method for achieving a reduction of fat mass in a subject, the method comprising administering a TrkB agonist antibody of Table 1, or a pharmaceutical composition comprising the antibody or antigen-binding fragment thereof, to the subject.

An eighth aspect of the invention provides for a method of promoting neuronal survival in a subject, the method comprising administering a therapeutically effective amount of a TrkB agonist antibody of Table 1, or a pharmaceutical composition comprising a therapeutically effective amount of the antibody or antigen-binding fragment thereof, to the subject.

In one embodiment, the methods described above may be achieved by administering an agonist anti-TrkB antibody or antigen-binding fragment thereof to a subject in need thereof, wherein the agonist anti-TrkB antibody comprises three heavy chain complementarity determining regions (CDRs) (HCDR1, HCDR2 and HCDR3) contained within a heavy chain variable region (HCVR) comprising an amino acid sequence as set forth in Table 1, or a substantially similar sequence thereof having at least 90% sequence identity thereto; and three light chain CDRs (LCDR1, LCDR2 and LCDR3) contained within a light chain variable region (LCVR) comprising an amino acid sequence as set forth in Table 1, or a substantially similar sequence thereof having at least 90% sequence identity thereto.

In one embodiment, the methods of the invention may be achieved by administering an agonist TrkB antibody of the invention, wherein the antibody or antigen-binding fragment thereof comprises three heavy chain CDRs (HCDR1, HCDR2 and HCDR3) contained within any one of the HCVR sequences selected from the group consisting of SEQ ID NOs: 2, 18, 34, 49, 59 and 68, or a substantially similar sequence thereof having at least 90% sequence identity thereto; and three light chain CDRs (LCDR1, LCDR2 and LCDR3) contained within any one of the LCVR sequences selected from the group consisting of SEQ ID NOs: 10, 26, 42, 53, 63 and 72, or a substantially similar sequence thereof having at least 90% sequence identity thereto.

In one embodiment, the antibody or antigen-binding fragment thereof comprises a HCVR having an amino acid sequence selected from the group consisting of SEQ ID NOs: 2, 18, 34, 49, 59 and 68.

In one embodiment, the antibody or antigen-binding fragment thereof comprises a LCVR having an amino acid sequence selected from the group consisting of SEQ ID NOs: 10, 26, 42, 53, 63 and 72.

In one embodiment, the antibody or antigen-binding fragment thereof comprises a HCVR having an amino acid sequence selected from the group consisting of SEQ ID NOs: 2, 18, 34, 49, 59 and 68; and a LCVR having an amino acid sequence selected from the group consisting of SEQ ID NOs: 10, 26, 42, 53, 63 and 72.

In one embodiment, the antibody or antigen-binding fragment thereof comprises the CDRs of a HCVR/LCVR amino acid sequence pair selected from the group consisting of SEQ ID NOS: 2/10, 18/26, 34/42, 49/53, 59/63 and 68/72.

In one embodiment, the antibody or antigen-binding fragment thereof comprises a HCVR/LCVR amino acid sequence pair selected from the group consisting of SEQ ID NOs: 2/10, 18/26, 34/42, 49/53, 59/63 and 68/72.

In one embodiment, the antibody or antigen-binding fragment thereof comprises:
(a) a HCDR1 domain having an amino acid sequence selected from the group consisting of SEQ ID NOs: 4, 20, 36, 50, 60 and 69;
(b) a HCDR2 domain having an amino acid sequence selected from the group consisting of SEQ ID NOs: 6, 22, 38, 51, 61 and 70;
(c) a HCDR3 domain having an amino acid sequence selected from the group consisting of SEQ ID NOs: 8, 24, 40, 52, 62 and 71;
(d) a LCDR1 domain having an amino acid sequence selected from the group consisting of SEQ ID NOs: 12, 28, 44, 54, 64 and 73;
(e) a LCDR2 domain having an amino acid sequence selected from the group consisting of SEQ ID NOs: 14, 30, 46, 55, 65 and 74; and
(f) a LCDR3 domain having an amino acid sequence selected from the group consisting of SEQ ID NOs: 16, 32, 48, 56, 66 and 75.

In one embodiment, the antibody or antigen-binding fragment thereof comprises a set of six CDRs (HCDR1-HCDR2-HCDR3-LCDR1-LCDR2-LCDR3) selected from the group consisting of: (a) SEQ ID NOs: 4-6-8-12-14-16; (b) SEQ ID NOs: 20-22-24-28-30-32; (c) SEQ ID NOs: 36-38-40-44-46-48; (d) SEQ ID NOs: 50-51-52-54-55-56; (e) SEQ ID NOs: 60-61-62-64-65-66; and (f) SEQ ID NOs: 69-70-71-73-74-75.

In one embodiment, the disease or disorder to be treated with an anti-TrkB antibody of the invention is obesity, and any complication resulting from obesity.

It is envisioned that any disease or disorder associated with TrkB activity or expression is amenable to treatment with an antibody of the invention. These diseases may include any disease in which cellular degradation is evident, such as in a neurodegenerative condition, or following a nerve injury.

Other embodiments will become apparent from a review of the ensuing detailed description.

### BRIEF DESCRIPTION OF THE FIGURES

FIG. 1. Shows western blots assessing total TRKB levels and phospho-TRKB levels in homozygous humanized TRKB mice at 1 hour, 4 hours, and 18 hours following direct hippocampal injection of TRKB agonist antibody H4H9816P2 or isotype control antibody.

FIG. 2. Shows that the TrkB agonist antibody, H4H9816P2, activated the downstream pathways of MAPK/ERK and PI3K/Akt. The figure shows western blots of phospho-TrkB, total TrkB, phospho-Akt, total AKT, phospho-ERK, and total ERK at 15 minutes and 2 hours after treatment of primary cortical neurons isolated from postnatal day 1 homozygous humanized TRKB mouse pups with various TrkB agonist antibodies or BDNF.

FIG. 3. Shows that three TrkB agonist antibodies dose dependently increased the survival of SH-SY5Y cells in vitro. The isotype control antibody had no effect on cell survival.

FIG. 4. Shows the pharmacokinetic profiles of the anti-TrkB agonist antibody H4H9816P2 in TrkB^{hu/hu} mice and Wild Type mice. Mice were administered a single 10 mg/kg sub-cutaneous dose on day 0. Concentrations of total H4H9816P2 in serum were measured using a Gyros immunoassay. Data points on post-dose 6 hours, 1, 2, 3, 6, 9, 16, 21 and 30 days indicate the mean concentration of antibody. Total antibody concentrations of H4H9816P2 are represented as black solid circles for TrkB^{hu/hu} mice and solid black squares for WildType mice. Data are plotted as mean±SD.

FIG. 5. Consists of FIG. 5A and FIG. 5B which show the ability of anti-mouse TrkB monoclonal antibodies to block interaction between mouse or rat TrkB and its ligand BDNF (Brain Derived Neurotrophic Factor). ELISA-based methods were used to assess the binding of (FIG. 5A with two graphs) mouse TrkB.hFc and (FIG. 5B with two graphs) rat TrkB.mmh to plate coated BDNF in presence of a range of concentrations of Anti-mouse TrkB and isotype control mAbs. The insert in (FIG. 5A with two graphs) shows the dose-response curve of mouse TrkB.hFc (REGN2277) binding to BDNF with an EC₅₀ value of 780 pM. The insert in (FIG. 5B with two graphs) shows the dose-response curve of rat TrkB.mmh (REGN1808) binding to BDNF with an EC₅₀ value of 2.2 nM. Molarity (M) indicates antibody concentration for mAbs. Error bars represent Standard Deviation.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Definitions

The expression “TrkB,” and the like, also known as “tropomyosin receptor kinase B”, refers to the human receptor (unless designated as being from another species) comprising the amino acid sequence as set forth in amino acid residues 32 through 430 of accession number NP_001018074.1. Human TrkB containing a myc-myc-hexahistidine tag is shown as SEQ ID NO: 76 (with amino acid residues 1-399 being human TrkB and amino acid residues 400-427 being the myc-myc-hexahistidine tag). Other formats containing human TrkB proteins are described herein, including SEQ ID NO: 77, which is human TrkB (residues 1-399) with a mouse Fc region (residues 400-632); and SEQ ID NO: 78, which is human TrkB (residues 1-399) with a human Fc region (residues 400-626). Mouse TrkB comprises the amino acid sequence as set forth in amino acid residues 32 through 429 of accession number NP_001020245. Mouse TrkB containing a myc-myc-hexahistidine tag is shown as SEQ ID NO: 79 (with amino acid residues 1-398 being mouse TrkB and amino acid residues 399-426 being the myc-myc-hexahistidine tag). Other formats containing mouse TrkB proteins are described herein, including SEQ ID NO: 80, which is mouse TrkB (residues 1-398) with a mouse Fc region (residues 399-631); and SEQ ID NO: 81, which is mouse TrkB (residues 1-398) with a human Fc region (residues 399-625. Rabbit TrkB comprises the amino acid sequence as set forth in amino acid residues 32 through 430 of accession number XP_002721319.1. Rabbit TrkB containing a myc-myc-hexahistidine tag is shown as SEQ ID NO: 82 (with amino acid residues 1-399 being rabbit TrkB and amino acid residues 400-427 being the myc-myc-hexahistidine tag). Other formats containing rabbit TrkB proteins are described herein, including SEQ ID NO: 83, which is rabbit TrkB (residues 1-399) with a mouse Fc region (residues 400-632). Rat TrkB comprises the amino acid sequence as set forth in amino acid residues 32 through 429 of accession number NP_036863.1. Rat TrkB containing a myc-myc-hexahistidine tag is shown as SEQ ID NO: 84 (with amino acid residues 1-398 being rat TrkB and amino acid residues 399-426 being the myc-myc-hexahistidine tag). Other formats containing rat TrkB proteins are described herein, including SEQ ID NO: 85, which is rat TrkB (residues 1-398) with a mouse Fc region (residues 399-631). Rhesus macaque (Macaca mulatta) TrkB is shown as SEQ ID NO: 95 (amino acids 32 through 838 of accession number NP_001248226.1) and cynomolgus monkey (Macaca fascicularis) TrkB is shown as SEQ ID NO: 96 (amino acids 32 through 838 of accession number XP_005582102.1).

The human “TrkA” protein is shown as SEQ ID NO: 86, with amino acids 1-375 being TrkA (amino acids 34-414 of accession number NP_001012331.1 with V263L, C300S), amino acids 376-378 being a GPG linker and amino acids 379-605 being a human Fc.

The human “TrkC” protein is shown as SEQ ID NO: 87, with amino acids 1-398 being TrkC (amino acids 32-429 of accession number NP_001012338.1) and amino acids 399-426 being a myc-myc-his tag.

The mouse “TrkC” protein is shown as SEQ ID NO: 88, with amino acids 1-398 being TrkC (amino acids 32-429 of accession number NP_032772.3) and amino acids 399-426 being a myc-myc-his tag.

The cynomolgus monkey “TrkC” protein is shown as SEQ ID NO: 89, with amino acids 1-398 being TrkC (amino acids 32-429 of accession number XP_015308837.1) and amino acids 399-426 being a myc-myc-his tag.

In certain instances, cell lines were prepared that expressed the TrkB proteins, including the ecto domain, as well as the transmembrane and cytoplasmic domains of the TrkB protein. For example, SEQ ID NO: 91 is the human TrkB protein containing all three domains contained within amino acids 32-822 of accession number NP_001018074.1) or Uniprot Q16620-1, with amino acids 1-398 being the ecto domain, and the transmembrane/cytoplasmic region defined by about amino acid residues 399-790. In one instance, a TrkB cell line was prepared which expressed mouse TrkB (amino acids 32-476 of accession number NP_032771.1; See also SEQ ID NO: 92). In another instance, a cell line was prepared that expressed a chimeric TrkB protein, with the ecto domain of mouse TrkB from amino acids 32-429 of accession number NP_001020245.1 (See also SEQ ID NO: 93) or Uniprot number P15209-1 and the human TrkB transmembrane and cytoplasmic domains (amino acids 431-822 of accession number NP_001018074.1 (See also SEQ ID NO: 91). A cell line was also prepared that expressed African Green Monkey (Chlorocebus sabaeus) TrkB (amino acids 32-822 of accession number XP_007967815.1(See also SEQ ID NO: 94)).

The term “brain derived neurotrophic factor” or “BDNF” refers to the ligand for TrkB and the amino acid sequence of BDNF is shown in SEQ ID NO: 90 (isoform A 1-120, with amino acids 129-247 of accession number NP_733928.1 with a Met added onto the N-terminal). In certain experiments described herein, the source of the BDNF is from R & D Systems, 248-BD/CF.

All references to proteins, polypeptides and protein fragments herein are intended to refer to the human version of the respective protein, polypeptide or protein fragment unless explicitly specified as being from a non-human species. Thus, the expression “ TrkB ” means human TrkB unless specified as being from a non-human species, e.g., “monkey TrkB,” “mouse TrkB,” “rat TrkB,” etc.

As used herein, the expression “anti-TrkB antibody” includes both monovalent antibodies with a single specificity, as well as bispecific antibodies comprising a first arm that binds TrkB and a second arm that binds a second (target) antigen, wherein the anti-TrkB arm comprises any of the HCVR/LCVR or CDR sequences as set forth in Table 1 herein. The expression “anti-TrkB antibody” also includes antibody-drug conjugates (ADCs) comprising an anti-TrkB antibody or antigen-binding portion thereof conjugated to a drug or toxin (i.e., cytotoxic agent). The expression “anti-TrkB antibody” also includes antibody-radionuclide conjugates (ARCs) comprising an anti-TrkB antibody or antigen-binding portion thereof conjugated to a radionuclide.

The term “anti-TrkB antibody”, as used herein, means any antigen-binding molecule or molecular complex comprising at least one complementarity determining region (CDR) that specifically binds to or interacts with TrkB or a portion of TrkB. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-TrkB antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes antigen-binding fragments of full length antibody molecules. The terms “antigen-binding portion” of an antibody, “antigen-binding fragment” of an antibody, and the like, as used herein, include any enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a Vu domain associated with a V_{L} domain, the V_{H} and V_{L} domains may be situated relative to one another in any suitable arrangement. For example, the variable region may be dimeric and contain V_{H}-V_{L}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

In certain instances, it may be desirable to antagonize TrkB, for example, for inhibiting the growth or proliferation of, for example, a neuronal tumor cell. However, the antibodies of the present invention act as agonist antibodies, which serve as enhancers of neuronal survival and as neuroprotectants. The antibodies of the present invention may function by enhancing the interaction between TrkB and its ligand, BDNF. Alternatively, the antibodies of the invention may mediate TrkB signaling through a mechanism that does not involve enhancing the TrkB interaction with its ligand

The term “human antibody”, as used herein, is intended to include non-naturally occurring human antibodies. The term includes antibodies that are recombinantly produced in a non-human mammal, or in cells of a non-human mammal. The term is not intended to include antibodies isolated from or generated in a human subject.

The antibodies of the invention may, in some embodiments, be recombinant and/or non-naturally occurring human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. In certain embodiments, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, C_{H}2 or C_{H}3 region, which may be desirable, for example, in production, to improve the yield of the desired antibody form.

The term “specifically binds”, or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to TrkB. Moreover, multi-specific antibodies that bind to TrkB protein and one or more additional antigens or a bi-specific that binds to two different regions of TrkB are nonetheless considered antibodies that “specifically bind”, as used herein.

The antibodies of the invention may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The anti-TrkB antibodies disclosed herein may comprise one or more amino acid substitutions, insertions and/or deletions in the framework and/or CDR regions of the heavy and light chain variable domains. Such mutations can be readily ascertained by comparing the amino acid sequences disclosed herein to sequences available from, for example, public antibody sequence databases. Once obtained, antibodies and antigen-binding fragments that contain one or more mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes anti-TrkB antibodies comprising variants of any of the HCVR, LCVR, and/or CDR amino acid sequences disclosed herein having one or more conservative substitutions. For example, the present invention includes anti-TrkB antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Table 1 herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

### Biological Characteristics of the Antibodies

The present invention includes anti-TrkB antibodies that bind human TrkB with a K_{D} of less than about 200 nM as measured by surface plasmon resonance at 25° C., or at 37° C. According to certain embodiments, the invention includes anti-TrkB antibodies that bind human TrkB with a K_{D} of less than about 600 pM, less than about 300 pM, less than about 200 pM, less than about 150 pM, less than about 100 pM, less than about 80 pM, less than about 50 pM, less than about 40 pM, less than about 30 pM, less than about 20 pM, less than about 10 pM, less than about 5 pM, less than about 3 pM, or less than about 1 pM.

The present invention includes anti-TrkB antibodies that bind human TrkB with a dissociative half life (t½) of greater than about 10 minutes as measured by surface plasmon resonance at 25° C., or 37° C. According to certain embodiments, the invention includes anti-TrkB antibodies that bind human TrkB with a t½ of greater than about 20 minutes, greater than about 50 minutes, greater than about 100 minutes, greater than about 120 minutes, greater than about 150 minutes, greater than about 300 minutes, greater than about 350 minutes, greater than about 400 minutes, greater than about 450 minutes, greater than about 500 minutes, greater than about 550 minutes, greater than about 600 minutes, greater than about 700 minutes, greater than about 800 minutes, greater than about 900 minutes, greater than about 1000 minutes, greater than about 1100 minutes, or greater than about 1200 minutes.

The present invention includes anti-TrkB antibodies that may or may not bind monkey TrkB, or mouse or rat TrkB. As used herein, an antibody “does not bind” a particular antigen (e.g., monkey, mouse or rat TrkB if the antibody, when tested in an antigen binding assay such as surface plasmon resonance exhibits a K_{D} of greater than about 1000 nM, or does not exhibit any antigen binding, in such an assay. Another assay format that can be used to determine whether an antibody binds or does not bind a particular antigen, according to this aspect of the invention, is ELISA.

The present invention includes anti-TrkB antibodies that activate human TrkB signaling in cells engineered to express an TrkB receptor with an EC₅₀ of less than about 100 pM. Using an assay format described in Example 5, or a substantially similar assay format, an EC₅₀ value can be calculated as the concentration of antibody required to activate TrkB-mediated signaling to the half-maximal signal observed. Thus, according to certain embodiments, the invention includes anti-TrkB antibodies that mediate human TrkB signaling in cells engineered to express a TrkB receptor in the presence or absence of BDNF with an EC₅₀ of less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, or less than about 5 pM, as measured using the assay format described in Example 5 herein or a substantially similar assay.

The present invention includes anti-TrkB antibodies that activate the TrkB receptor as shown by TrkB phosphorylation following direct hippocampal injection in mice humanized to express the human TrkB receptor, as shown in Example 6.

The present invention includes anti-TrkB antibodies that promote weight loss in mice humanized to express the human TrkB receptor. The antibodies of the invention also serve to promote loss of fat mass and increase locomotor activity in these mice, while decreasing food and water intake (see Example 7)

The antibodies of the invention promote survival of retinal ganglion cells (RGCs) in rats humanized to express the human TrkB receptor, when tested in an optic nerve transection model. See Example 8.

The antibodies of the invention activate the downstream pathways MAPK/ERK and PI3K/Akt, as shown by exposure of primary mouse cortical neurons obtained from humanized TrkB mice to the antibodies of the invention. (See Example 9).

The agonist anti-TrkB antibodies of the invention also promote survival of SH-SY5Y cells in a dose dependent fashion, as shown in Example 10.

The present invention includes anti-TrkB antibodies that block TrkB binding to BDNF with an IC₅₀ of less than about 5 nM. For example, as shown in Example 12, all three antibodies tested blocked >50% of mouse or rat TrkB binding to BDNF. Using the assay format described in Example 12, or a substantially similar assay format, an IC₅₀ value can be calculated as the concentration of antibody required to block TrkB binding to BDNF when compared to the maximal signal observed in the absence of antibody. Thus, according to certain embodiments, the invention includes anti-TrkB antibodies that block TrkB binding to BDNF with an IC₅₀ of less than about 5 nM, less than about 4 nM, less than about 3 nM, less than about 2 nM, less than about 1 nM, less than about 900 pM, less than about 800 pM, less than about 700 pM, less than about 600 pM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, or less than about 20 pM, as measured using the assay format described in Example 12 herein or a substantially similar assay. In one embodiment, the TrkB antibodies of the invention block TrkB binding to BDNF with an IC₅₀ ranging from about 180 pM to about 4 nM.

A binding characteristic of an antibody of the invention (e.g., any of the binding characteristics mentioned herein above), when disclosed in term of being “measured by surface plasmon resonance” means that the relevant binding characteristic pertaining to the interaction between the antibody and the antigen are measured using a surface plasmon resonance instrument (e.g., a Biacore® instrument, GE Healthcare) using standard Biacore assay conditions as illustrated in Examples 3 and 4 herein, or substantially similar assay format. In certain embodiments, the binding parameters are measured at 25° C., while in other embodiments, the binding parameters are measured at 37° C.

The present invention includes antibodies or antigen-binding fragments thereof that specifically bind TrkB, comprising an HCVR and/or an LCVR comprising an amino acid sequence selected from any of the HCVR and/or LCVR amino acid sequences listed in Table 1.

The antibodies of the present invention may possess one or more of the aforementioned biological characteristics, or any combination thereof. The foregoing list of biological characteristics of the antibodies of the invention is not intended to be exhaustive. Other biological characteristics of the antibodies of the present invention will be evident to a person of ordinary skill in the art from a review of the present disclosure including the working Examples herein.

### Epitope Mapping and Related Technologies

The epitope to which the antibodies of the present invention bind may consist of a single contiguous sequence of 3 or more (e.g., 3, 4, 5, 6, 7, 8, 9, 10, 11, 12, 13, 14, 15, 16, 17, 18, 19, 20 or more) amino acids of a TrkB protein. Alternatively, the epitope may consist of a plurality of non-contiguous amino acids (or amino acid sequences) of TrkB. In some embodiments, the epitope is located on or near a surface of TrkB, for example, in the domain that interacts with its ligand, BDNF. In other embodiments, the epitope is located on or near a surface of TrkB that does not interact with the TrkB ligand, e.g., at a location on the surface of TrkB at which an antibody, when bound to such an epitope, does not interfere with the interaction between TrkB and its ligand.

Various techniques known to persons of ordinary skill in the art can be used to determine whether an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., NY), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A.

The present invention includes anti-TrkB antibodies that bind to the same epitope as any of the specific exemplary antibodies described herein (e.g. antibodies comprising any of the amino acid sequences as set forth in Table 1 herein). Likewise, the present invention also includes anti-TrkB antibodies that compete for binding to TrkB with any of the specific exemplary antibodies described herein (e.g. antibodies comprising any of the amino acid sequences as set forth in Table 1 herein).

One can easily determine whether an antibody binds to the same epitope as, or competes for binding with, a reference anti-TrkB antibody by using routine methods known in the art and exemplified herein. For example, to determine if a test antibody binds to the same epitope as a reference anti-TrkB antibody of the invention, the reference antibody is allowed to bind to an TrkB protein. Next, the ability of a test antibody to bind to the TrkB molecule is assessed. If the test antibody is able to bind to TrkB following saturation binding with the reference anti-TrkB antibody, it can be concluded that the test antibody binds to a different epitope than the reference anti-TrkB antibody. On the other hand, if the test antibody is not able to bind to the TrkB molecule following saturation binding with the reference anti-TrkB antibody, then the test antibody may bind to the same epitope as the epitope bound by the reference anti-TrkB antibody of the invention. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present invention, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990:50:1495-1502). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding (or cross-competes for binding) with a reference anti-TrkB antibody, the above-described binding methodology is performed in two orientations: In a first orientation, the reference antibody is allowed to bind to an TrkB protein under saturating conditions followed by assessment of binding of the test antibody to the TrkB molecule. In a second orientation, the test antibody is allowed to bind to a TrkB molecule under saturating conditions followed by assessment of binding of the reference antibody to the TrkB molecule. If, in both orientations, only the first (saturating) antibody is capable of binding to the TrkB molecule, then it is concluded that the test antibody and the reference antibody compete for binding to TrkB (see, e.g., the assay format described in Example 4 herein, in which TrkB protein is captured onto sensor tips and the TrkB-coated sensor tips are treated with a reference antibody [mAb-1] and a test anti-TrkB antibody [mAb-2] sequentially and in both binding orders). As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

### Preparation of Human Antibodies

The anti-TrkB antibodies of the present invention can be fully human but non-naturally occurring, antibodies. Methods for generating monoclonal antibodies, including fully human monoclonal antibodies are known in the art. Any such known methods can be used in the context of the present invention to make human antibodies that specifically bind to human TrkB.

Using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to an allergen are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

As described in the experimental section below, the high affinity chimeric antibodies, which are isolated having a human variable region and a mouse constant region, are characterized and selected for desirable characteristics, including affinity, selectivity, epitope, etc. The mouse constant regions are then replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In certain embodiments, it may be desirable to test anti-human TrkB antibodies in mice or rats that have been engineered to express a human TrkB receptor. These mice or rats may be beneficial in circumstances wherein the anti-TrkB antibodies may only bind human TrkB, but will not cross react with mouse or rat TrkB. Certain examples in the present invention were carried out using mice and rats that were genetically modified to express the human trkB. Any method known to those skilled in the art may be used for generating such TrkB humanized mice and rats.

In general, the antibodies of the instant invention possess very high affinities, typically possessing K_{D} of from about 10⁻¹² through about 10⁻⁹ M, when measured by binding to antigen either immobilized on solid phase or in solution phase.

### Bioequivalents

The anti-TrkB antibodies and antibody fragments of the present invention encompass proteins having amino acid sequences that vary from those of the described antibodies but that retain the ability to bind human TrkB. Such variant antibodies and antibody fragments comprise one or more additions, deletions, or substitutions of amino acids when compared to parent sequence, but exhibit biological activity that is essentially equivalent to that of the described antibodies. Likewise, the anti-TrkB antibody-encoding DNA sequences of the present invention encompass sequences that comprise one or more additions, deletions, or substitutions of nucleotides when compared to the disclosed sequence, but that encode an anti-TrkB antibody or antibody fragment that is essentially bioequivalent to an anti-TrkB antibody or antibody fragment of the invention. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-TrkB antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-TrkB antibody variants comprising amino acid changes which modify the glycosylation characteristics of the antibodies, e.g., mutations which eliminate or remove glycosylation.

### Species Selectivity and Species Cross-Reactivity

The present invention, according to certain embodiments, provides anti-TrkB antibodies that bind to human TrkB but not to TrkB from other species. The present invention also includes anti-TrkB antibodies that bind to human TrkB and to TrkB from one or more non-human species. For example, the anti-TrkB antibodies of the invention may bind to human TrkB and may bind or not bind, as the case may be, to one or more of mouse, rat, guinea pig, hamster, gerbil, pig, cat, dog, rabbit, goat, sheep, cow, horse, camel, cynomologous, marmoset, rhesus or chimpanzee TrkB. According to certain exemplary embodiments of the present invention, anti-TrkB antibodies are provided which specifically bind human TrkB but do not bind, or bind only weakly, to mouse or rat TrkB.

### Multispecific Antibodies

The antibodies of the present invention may be monospecific or multispecific (e.g., bispecific). Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The anti-TrkB antibodies of the present invention can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity.

The present invention includes bispecific antibodies wherein one arm of an immunoglobulin binds human TrkB, and the other arm of the immunoglobulin is specific for a second antigen. The TrkB-binding arm can comprise any of the HCVR/LCVR or CDR amino acid sequences as set forth in Table 1 herein.

An exemplary bispecific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V821 (by IMGT; Q355R, N384S, K392N, V397M, R409K, E419Q, and V422I by EU) in the case of IgG4 antibodies. Variations on the bispecific antibody format described above are contemplated within the scope of the present invention.

Other exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

### Therapeutic Formulation and Administration

The invention provides pharmaceutical compositions comprising the anti-TrkB antibodies or antigen-binding fragments thereof of the present invention. The pharmaceutical compositions of the invention are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody administered to a patient may vary depending upon the age and the size of the patient, target disease, conditions, route of administration, and the like. The preferred dose is typically calculated according to body weight or body surface area. In an adult patient, it may be advantageous to intravenously administer the antibody of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering anti-TrkB antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intravitreal, intraocular, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™M Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; sec, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, intravitreal, intraocular, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### Therapeutic Uses of the Antibodies

The present invention includes methods comprising administering to a subject in need thereof a therapeutic composition comprising an anti-TrkB antibody (e.g., an anti-TrkB antibody comprising any of the HCVR/LCVR or CDR sequences as set forth in Table 11 herein). The therapeutic composition can comprise any one or more of the anti-TrkB antibodies or antigen-binding fragments thereof disclosed herein, and a pharmaceutically acceptable carrier or diluent.

The antibodies of the invention are useful, inter alia, for the treatment, prevention and/or amelioration of any disease or disorder associated with or mediated by TrkB expression or activity. The TrkB agonist antibodies of the invention may be used to improve nerve function and may be used to treat or prevent any disease or condition that is characterized in part by cellular degradation, in particular by nerve cell injury or nerve cell degeneration, e.g. an acute nervous system injury or a chronic neurodegenerative disease.

The present invention includes methods of treating or preventing a disease or disorder of the eye by administering to a patient in need of such treatment an anti-TrkB antibody or antigen-binding fragment thereof as disclosed elsewhere herein.

In one embodiment, the anti-TrkB antibodies of the invention may provide a method of preventing injury or death of retinal neurons. In one embodiment, the anti-TrkB antibodies of the invention may provide a method of treating pathological diseases wherein degeneration of the retina occurs. In one embodiment, the anti-TrkB antibodies of the invention may provide a method of treating the eye prior to or following ocular surgery, exposure to light or other environmental trauma thereby preventing degeneration of retinal cells. In one embodiment, the anti-TrkB antibodies of the invention may provide a method of preventing photoreceptor injury and degeneration in the eye. In one embodiment, the anti-TrkB antibodies of the invention may provide a method of protecting retinal neurons without the induction of side effects. In one embodiment, the anti-TrkB antibodies of the invention may provide a method of allowing injured photoreceptors to recover or regenerate.

In certain embodiments, the eye diseases treatable by using one or more of the anti-TrkB antibodies of the invention may be selected from the group consisting of glaucoma, diabetic retinopathy, age-related macular degeneration or other maculopathies, ischemic optic neuropathy, optic neuritis, retinal ischemia, photoreceptor degeneration, retinitis pigmentosa, Leber Congenital Amaurosis, Leber's hereditary optic neuropathy, Usher Syndrome, Stargardt disease, and retinal artery or vein occlusions.

Other pathological conditions treatable with one or more anti-TrkB antibodies of the invention include retinal detachment, photic retinopathies, surgery-induced retinopathies (either mechanically or light-induced), toxic retinopathies, retinopathy of prematurity, viral retinopathies such as CMV or HIV retinopathy related to AIDS; uveitis; ischemic retinopathies due to venous or arterial occlusion or other vascular disorder, retinopathies due to trauma or penetrating lesions of the eye, peripheral vitreoretinopathy or inherited retinal degenerations.

In one embodiment, the anti-TrkB antibodies of the invention may be formulated for intraocular or intravitreal delivery.

The present invention also provides methods for treating other central or peripheral nervous system diseases or disorders, such as stroke, or traumatic brain injury. Furthermore, since the antibodies of the present invention act to promote neuronal survival and act as neuroprotectants, any one or more of these agonist antibodies may prove to be beneficial in treating a patient suffering from a nervous system disease or disorder whereby neuronal survival is of prime importance in recovery or repair of the cellular damage caused by an injury to the nervous system, or caused by a disease that has a major effect on the nervous system, including neurodegenerative diseases.

In the context of the methods of treatment described herein, the anti-TrkB antibody may be administered as a monotherapy (i.e., as the only therapeutic agent) or in combination with one or more additional therapeutic agents.

### Combination Therapies and Formulations

The present invention includes compositions and therapeutic formulations comprising any of the anti-TrkB antibodies described herein in combination with one or more additional therapeutically active components, and methods of treatment comprising administering such combinations to subjects in need thereof.

The anti-TrkB antibodies of the present invention may be co-formulated with and/or administered in combination with one or more additional therapeutically active component(s) selected from the group consisting of: a drug that helps to lower intraocular pressure (an IOP lowering drug), a neurotrophin, and an antagonist of vascular endothelial growth factor (VEGF), such as a VEGF trap, e.g. aflibercept (EYLEAR). Other medications that may be combined with the TrkB antibodies of the invention include, but are not limited to, a prostaglandin analog (e.g. ZIOPTAN™, XALATAN®), a beta blocker, (e.g. TIMOPTIC XE®, ISTALOL®, BETOPTIC®S); an alpha-2 adrenergic agonist (e.g. apraclonidine), carbonic anhydrase inhibitors (e.g. TRUSOPT®, AZOPT®), a cholinergic agent (e.g. ISOPTO®CARPINE, PILOPINE HS® gel), or a combined therapeutic (a beta blocker plus a carbonic anhydrase inhibitor, e.g. COMBIGAN™, COSOPT®).

The anti-TrkB antibodies of the invention may also be administered and/or co-formulated in combination with antivirals, antibiotics, analgesics, antioxidants, COX inhibitors, and/or NSAIDs. The anti-TrkB antibodies may also be used in conjunction with other types of therapy including stem cell therapy, glaucoma filtration surgery, laser surgery, or gene therapy.

The additional therapeutically active component(s), e.g., any of the agents listed above or derivatives thereof, may be administered just prior to, concurrent with, or shortly after the administration of an anti-TrkB antibody of the present invention; (for purposes of the present disclosure, such administration regimens are considered the administration of an anti-TrkB antibody “in combination with” an additional therapeutically active component). The present invention includes pharmaceutical compositions in which an anti-TrkB antibody of the present invention is co-formulated with one or more of the additional therapeutically active component(s) as described elsewhere herein.

### Administration Regimens

According to certain embodiments of the present invention, multiple doses of an anti-TrkB antibody (or a pharmaceutical composition comprising a combination of an anti-TrkB antibody and any of the additional therapeutically active agents mentioned herein) may be administered to a subject over a defined time course. The methods according to this aspect of the invention comprise sequentially administering to a subject multiple doses of an anti-TrkB antibody of the invention. As used herein, “sequentially administering” means that each dose of anti-TrkB antibody is administered to the subject at a different point in time, e.g., on different days separated by a predetermined interval (e.g., hours, days, weeks or months). The present invention includes methods which comprise sequentially administering to the patient a single initial dose of an anti-TrkB antibody, followed by one or more secondary doses of the anti-TrkB antibody, and optionally followed by one or more tertiary doses of the anti-TrkB antibody.

The terms “initial dose,” “secondary doses,” and “tertiary doses,” refer to the temporal sequence of administration of the anti-TrkB antibody of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-TrkB antibody, but generally may differ from one another in terms of frequency of administration. In certain embodiments, however, the amount of anti-TrkB antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of anti-TrkB antibody, which is administered to a patient prior to the administration of the very next dose in the sequence with no intervening doses.

The methods according to this aspect of the invention may comprise administering to a patient any number of secondary and/or tertiary doses of an anti-TrkB antibody. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient. The administration regimen may be carried out indefinitely over the lifetime of a particular subject, or until such treatment is no longer therapeutically needed or advantageous.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the invention, the frequency at which the secondary and/or tertiary doses are administered to a patient can vary over the course of the treatment regimen. The frequency of administration may also be adjusted during the course of treatment by a physician depending on the needs of the individual patient following clinical examination.

The present invention includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this aspect of the invention, if the loading doses are administered at a frequency of once a month, then the maintenance doses may be administered to the patient once every six weeks, once every two months, once every three months, etc.

### Diagnostic Uses of the Antibodies

The anti-TrkB antibodies of the present invention may also be used to detect and/or measure TrkB, or TrkB-expressing cells in a sample, e.g., for diagnostic purposes. For example, an anti-TrkB antibody, or fragment thereof, may be used to diagnose a condition or disease characterized by aberrant expression (e.g., over-expression, under-expression, lack of expression, etc.) of TrkB. Exemplary diagnostic assays for TrkB may comprise, e.g., contacting a sample, obtained from a patient, with an anti-TrkB antibody of the invention, wherein the anti-TrkB antibody is labeled with a detectable label or reporter molecule. Alternatively, an unlabeled anti-TrkB antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure TrkB in a sample include enzyme-linked immunosorbent assay (ELISA), radioimmunoassay (RIA), and fluorescence-activated cell sorting (FACS).

Samples that can be used in TrkB diagnostic assays according to the present invention include any tissue or fluid sample obtainable from a patient, which contains detectable quantities of TrkB protein, or fragments thereof, under normal or pathological conditions. Generally, levels of TrkB in a particular sample obtained from a healthy patient (e.g., a patient not afflicted with a disease or condition associated with abnormal TrkB levels or activity) will be measured to initially establish a baseline, or standard, level of TrkB. This baseline level of TrkB can then be compared against the levels of TrkB measured in samples obtained from individuals suspected of having a TrkB related disease or condition.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Human Antibodies to TrkB

Human antibodies to TrkB were generated in a mouse comprising DNA encoding human immunoglobulin heavy and kappa light chain variable regions. In one embodiment, the human antibodies were generated in a VELOCIMMUNE® mouse. In one embodiment, VelocImmune® (VI) mice were immunized with human TrkB(ecto)mFc (SEQ ID NO: 77). In one embodiment, VelocImmune® (VI) mice were immunized with mouse TrkB(ecto)mFc (SEQ ID NO: 80). The antibody immune response was monitored by TrkB specific immunoassay. For example, sera were assayed for specific antibody titers to purified full-length TrkB. Antibody-producing clones were isolated using both B-cell Sorting Technology (BST) and hybridoma methods. For example, when a desired immune response was achieved, splenocytes were harvested and fused with mouse myeloma cells to preserve their viability and form hybridoma cell lines. The hybridoma cell lines were screened and selected to identify cell lines that produce TrkB-specific antibodies. Certain anti-mouse TrkB antibodies were generated this way and are designated M2aM14173N, M2aM14178N and M2aM14179N.

Anti-TrkB antibodies were also isolated directly from antigen-positive mouse B cells without fusion to myeloma cells, as described in U.S. Pat. No. 7,582,298, herein specifically incorporated by reference in its entirety. Using this method, several fully human anti-TrkB antibodies (i.e., antibodies possessing human variable domains and human constant domains) were obtained; exemplary antibodies generated in this manner were designated as H4H9780P, H4H9814P and H4H9816P2.

The biological properties of the exemplary antibodies generated in accordance with the methods of this Example are described in detail in the Examples set forth below.

### Example 2: Heavy and Light Chain Variable Region Amino Acid and Nucleotide Sequences

Table la sets forth the amino acid sequence identifiers of the heavy and light chain variable regions and CDRs of selected anti-TrkB antibodies of the invention. Table 1b sets forth the amino acid sequence identifiers for the full length heavy and light chains of selected anti-TrkB antibodies of the invention. The corresponding nucleic acid sequence identifiers for selected anti-TrkB antibodies of the invention are set forth in Table 2.

TABLE 1a
Amino Acid Sequence Identifiers

| Ab Name | HCVR | HCDR1 | HCDR2 | HCDR3 | LCVR | LCDR1 | LCDR2 | LCDR3 | mIgG2a | Constant LC |
|---|---|---|---|---|---|---|---|---|---|---|
| H4H9780P | 2 | 4 | 6 | 8 | 10 | 12 | 14 | 16 | | |
| H4H9814P | 18 | 20 | 22 | 24 | 26 | 28 | 30 | 32 | | |
| H4H9816P2 | 34 | 36 | 38 | 40 | 42 | 44 | 46 | 48 | | |
| M2aM14173N | 49 | 50 | 51 | 52 | 53 | 54 | 55 | 56 | 57 | 58 |
| M2aM14178N | 59 | 60 | 61 | 62 | 63 | 64 | 65 | 66 | 57 | 67 |
| M2aM14179N | 68 | 69 | 70 | 71 | 72 | 73 | 74 | 75 | 57 | 58 |

TABLE 1b

| | Full length | Full length |
|---|---|---|
| Ab Name | heavy chain | light chain |
| H4H9780P | 99 | 100 |
| H4H9814P | 101 | 102 |
| H4H9816P2 | 103 | 104 |

TABLE 2
Nucleic Acid Sequence Identifiers

| Ab Name | VH | HCDR1 | HCDR2 | HCDR3 | VK | LCDR1 | LCDR2 | LCDR3 |
|---|---|---|---|---|---|---|---|---|
| H4H9780P | 1 | 3 | 5 | 7 | 9 | 11 | 13 | 15 |
| H4H9814P | 17 | 19 | 21 | 23 | 25 | 27 | 29 | 31 |
| H4H9816P2 | 33 | 35 | 37 | 39 | 41 | 43 | 45 | 47 |

Antibodies are typically referred to herein according to the following nomenclature: Fc prefix (e.g. “H4H,” “H2M,” etc.), followed by a numerical identifier (e.g. “9780,” “9816,” etc., as shown in Table 1 or 2), followed by a “P,” “P2,” or “N” suffix. The H4H prefix on the antibody designations indicate the particular Fc region isotype of the antibody. Thus, according to this nomenclature, an antibody may be referred to herein as, e.g., “ H4H9780P”, which indicates a human IgG4 Fc region and M2aM14179N, for example, indicates a mouse IgG2a Fc region. Variable regions are fully human if denoted by the first ‘H’ in the antibody designation. An ‘M’ prefix designates a mouse variable region. As will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs)—which are indicated by the numerical identifiers shown in Table 1 or 2—will remain the same, and the binding properties to antigen are expected to be identical or substantially similar regardless of the nature of the Fc domain.

### Example 3. Biacore Binding Kinetics of Anti-TrkB Monoclonal Antibodies Binding to Different TrkB Reagents Measured at 25° C. and 37° C.

Equilibrium dissociation constants (K_{D} values) for TrkB binding to purified anti-TrkB monoclonal antibodies were determined using a real-time surface plasmon resonance biosensor using a Biacore 4000 instrument. All binding studies were performed in 10 mM Hepes pH 7.4, 150 mM NaCl, 3 mM EDTA, and 0.05%v/v Surfactant Tween-20 (HBS-ET running buffer) at 25° C. and 37° C. The Biacore sensor surface was first derivatized by amine coupling with F(ab′)2 fragment goat anti-human Fcγ specific polyclonal antibody (Jackson ImmunoResearch Laboratories, #109-006-098) or rabbit anti-mouse Fc polyclonal antibody (GE Healthcare #BR-1008-38) to capture anti-TrkB monoclonal antibodies. Binding studies were performed on following TrkB reagents; human TrkB extracellular domain expressed with a C-terminal myc-myc-hexahistidine tag (hTRKB.mmH; SEQ ID NO: 76; Accession number NP_001018074.1), mouse TrkB extracellular domain expressed with a C-terminal myc-myc-hexahistidine tag (mTRKB.mmH; SEQ ID NO: 79; Accession number NP_001020245), rat TrkB extracellular domain expressed with a C-terminal myc-myc-hexahistidine tag (rTRKB.mmH; SEQ ID NO: 84; Accession number NP_036863.1), and human TrkB extracellular domain expressed with a C-terminal mouse IgG2a Fc tag (hTrkB-mFc; SEQ ID NO: 77; Accession number NP_001018074.1). Different concentrations of TrkB reagents were first prepared in HBS-ET running buffer (100 nM-1.23 nM; 3-fold serial dilution) and were injected over anti-human Fc captured anti-TrkB monoclonal antibody surface for 4 minutes at a flow rate of 30 μL/minute, while the dissociation of monoclonal antibody bound TrkB reagent was monitored for 10 minutes in HBS-ET running buffer. Kinetic association (kₐ) and dissociation (k_{D}) rate constants were determined by fitting the real-time binding sensorgrams to a 1:1 binding model with mass transport limitation using Scrubber 2.0c curve-fitting software. Binding dissociation equilibrium constants (K_{D}) and dissociative half-lives (t½) were calculated from the kinetic rate constants as:

$${K}_{D} ( M ) = \frac{kd}{ka} , and {t}^{1 / 2} ( min ) = \frac{ln ( 2 )}{60 \times kd}$$

Binding kinetic parameters for hTrkB.mmH, mTrkB.mmH, rTrkB.mmH, or hTrkB-mFc binding to different anti-TrkB monoclonal antibodies of the invention at 25° C. and 37° C. are shown in Tables 3 through 10.

### Summary of Results

At 25° C., anti-TrkB monoclonal antibodies bound to hTrkB.mmH with KD values ranging from 545 pM to 41.3 nM, as shown in Table 3. At 37° C., monoclonal antibodies bound to hTrkB.mmH with KD values ranging from 2.28 nM to 135 nM, as shown in Table 4.

At 25° C., anti-TrkB monoclonal antibodies bound to hTrkB-mFc with KD values ranging from 31.1 pM to 4.48 nM, as shown in Table 5. At 37° C., anti-TrkB monoclonal antibodies bound to hTrkB-mFc with KD values ranging from 73.3 pM to 3.46 nM, as shown in Table 6.

At 25° C., the comparator anti-TrkB monoclonal antibody, referred to herein as H1M8037C, (See US2010/0196390, the antibody designated as C2; See also SEQ ID NOs: 97 and 98 for the comparator heavy and light chain, respectively, amino acid sequences) bound to mTrkB.mmH with a KD value of 40.8 nM, as shown in Table 7. The anti-TrkB antibodies of the invention did not bind to mTrkB.mmH at 25° C., as shown in Table 7. At 37° C., the comparator anti-TrkB monoclonal antibody bound to mTrkB.mmH with a KD value of 94.1nM, as shown in Table 8. The anti-TrkB antibodies of the invention did not bind to mTrkB.mmH at 37° C., as shown in Table 8.

At 25° C., the comparator anti-TrkB monoclonal antibody bound to rTrkB.mmH with a KD value of 31.8 nM, as shown in Table 9. The anti-TrkB antibodies of the invention did not bind to rTrkB.mmH at 25° C., as shown in Table 9. At 37° C., the comparator anti-TrkB monoclonal antibody bound to rTrkB.mmH with a KD value of 87.5 nM, as shown in Table 10. The anti-TrkB antibodies of the invention did not bind to rTrkB.mmH at 37° C., as shown in Table 10.

TABLE 3
Binding kinetics parameters of hTrkB.mmH binding
to TrkB monoclonal antibodies at 25° C.

| | Amount of | 100 nM | | | | |
|---|---|---|---|---|---|---|
| | Analyte | hTrkB.mmH | | | | |
| Captured | Captured | Bound | kₐ | k_{d} | K_{D} | t_{1/2} |
| Analyte | (RU) | (RU) | (1/Ms) | (1/s) | (M) | (min) |
| H4H9780P | 116 ± 2.8 | 20 | 4.00E+04 | 8.67E−04 | 2.17E−08 | 13 |
| H4H9814P | 93 ± 2.1 | 63 | 1.26E+06 | 6.86E−04 | 5.45E−10 | 17 |
| H4H9816P2 | 138 ± 1.5 | 30 | 6.14E+04 | 8.45E−04 | 1.38E−08 | 14 |
| H1M8037C* | 757 | 23 | 4.65E+04 | 1.92E−03 | 4.13E−08 | 6 |
| Comparator | | | | | | |

*indicates that mAb was captured using anti-mFc immobilized surface

TABLE 4
Binding kinetics parameters of hTrkB.mmH binding
to TrkB monoclonal antibodies at 37° C.

| | Amount of | 100 nM | | | | |
|---|---|---|---|---|---|---|
| | Analyte | hTrkB.mmH | | | | |
| Captured | Captured | Bound | kₐ | k_{d} | K_{D} | t_{1/2} |
| Analyte | (RU) | (RU) | (1/Ms) | (1/s) | (M) | (min) |
| H4H9780P | 64 ± 1.9 | 14 | 5.71E+04 | 3.64E−03 | 6.38E−08 | 3.2 |
| H4H9814P | 52 ± 1.2 | 32 | 2.05E+06 | 4.68E−03 | 2.28E−09 | 2.5 |
| H4H9816P2 | 80 ± 2.3 | 12 | 5.28E+04 | 3.74E−03 | 7.08E−08 | 3.1 |
| H1M8037C* | 815.11 | 28 | 6.13E+04 | 8.31E−03 | 1.35E−07 | 1.4 |
| Comparator | | | | | | |

*indicates that mAb was captured using anti-mFc immobilized surface

TABLE 5
Binding kinetics parameters of hTrkB-mFc binding
to TrkB monoclonal antibodies at 25° C.

| | Amount of | 100 nM | | | | |
|---|---|---|---|---|---|---|
| | Analyte | hTrkB-mFc | | | | |
| Captured | Captured | Bound | kₐ | k_{d} | K_{D} | t_{1/2} |
| Analyte | (RU) | (RU) | (1/Ms) | (1/s) | (M) | (min) |
| H4H9780P | 105 ± 1.6 | 14 | 3.74E+04 | 1.68E−04 | 4.48E−09 | 69 |
| H4H9814P | 83 ± 1.3 | 69 | 1.91E+06 | 5.95E−05 | 3.11E−11 | 194 |
| H4H9816P2 | 131 ± 1 | 25 | 6.52E+04 | 7.98E−05 | 1.22E−09 | 145 |
| H1M8037C* | 753 | 41 | 8.80E+04 | 9.55E−05 | 1.09E−09 | 121 |
| Comparator | | | | | | |

*indicates that mAb was captured using anti-mFc immobilized surface

TABLE 6
Binding kinetics parameters of hTrkB.mFc binding
to TrkB monoclonal antibodies at 37° C.

| | Amount of | 100 nM | | | | |
|---|---|---|---|---|---|---|
| | Analyte | hTrkB-mFc | | | | |
| Captured | Captured | Bound | kₐ | k_{d} | K_{D} | t_{1/2} |
| Analyte | (RU) | (RU) | (1/Ms) | (1/s) | (M) | (min) |
| H4H9780P | 56 ± 1 | 14 | 6.65E+04 | 2.22E−04 | 3.33E−09 | 52 |
| H4H9814P | 46 ± 1 | 42 | 2.83E+06 | 2.08E−04 | 7.33E−11 | 56 |
| H4H9816P2 | 71 ± 1.4 | 14 | 6.95E+04 | 2.40E−04 | 3.46E−09 | 48 |
| H1M8037C* | 814.46 | 58 | 2.35E+05 | 2.92E−04 | 1.24E−09 | 40 |
| Comparator | | | | | | |

*indicates that mAb was captured using anti-mFc immobilized surface

TABLE 7
Binding kinetics parameters of mTrkB.mmH binding
to TrkB monoclonal antibodies at 25° C.

| | Amount of | 100 nM | | | | |
|---|---|---|---|---|---|---|
| | Analyte | mTrkB.mmH | | | | |
| Captured | Captured | Bound | kₐ | k_{d} | K_{D} | t_{1/2} |
| Analyte | (RU) | (RU) | (1/Ms) | (1/s) | (M) | (min) |
| H4H9780P | 111 ± 0.7 | −1 | NB | NB | NB | NB |
| H4H9814P | 88 ± 0.7 | −2 | NB | NB | NB | NB |
| H4H9816P2 | 135 ± 0.6 | 0 | NB | NB | NB | NB |
| H1M8037C* | 755 | 26 | 4.12E+04 | 1.68E−03 | 4.08E−08 | 7 |
| Comparator | | | | | | |

*indicates that mAb was captured using anti-mFc immobilized surface

TABLE 8
Binding kinetics parameters of mTrkB.mmH binding
to TrkB monoclonal antibodies at 37° C.

| | Amount of | 100 nM | | | | |
|---|---|---|---|---|---|---|
| | Analyte | mTrkBmmH | | | | |
| Captured | Captured | Bound | kₐ | k_{d} | K_{D} | t_{1/2} |
| Analyte | (RU) | (RU) | (1/Ms) | (1/s) | (M) | (min) |
| H4H9780P | 60 ± 0.7 | 1 | NB | NB | NB | NB |
| H4H9814P | 49 ± 0.6 | 0 | NB | NB | NB | NB |
| H4H9816P2 | 75 ± 0.9 | 0 | NB | NB | NB | NB |
| H1M8037C* | 815.95 | 31 | 7.40E+04 | 6.96E−03 | 9.41E−08 | 1.7 |
| Comparator | | | | | | |

*indicates that mAb was captured using anti-mFc immobilized surface

TABLE 9
Binding kinetics parameters of rTrkB.mmH binding
to TrkB monoclonal antibodies at 25° C.

| | Amount of | 100 nM | | | | |
|---|---|---|---|---|---|---|
| | Analyte | rTrkB.mmH | | | | |
| Captured | Captured | Bound | kₐ | k_{d} | K_{D} | t_{1/2} |
| Analyte | (RU) | (RU) | (1/Ms) | (1/s) | (M) | (min) |
| H4H9780P | 108 ± 0.7 | −1 | NB | NB | NB | NB |
| H4H9814P | 86 ± 0.4 | −1 | NB | NB | NB | NB |
| H4H9816P2 | 134 ± 0.3 | 0 | NB | NB | NB | NB |
| H1M8037C* | 756 | 26 | 5.16E+04 | 1.60E−03 | 3.10E−08 | 7 |
| Comparator | | | | | | |

*indicates that mAb was captured using anti-mFc immobilized surface

TABLE 10
Binding kinetics parameters of rTrkB.mmH binding
to TrkB monoclonal antibodies at 37° C.

| | Amount of | 100 nM | | | | |
|---|---|---|---|---|---|---|
| | Analyte | rTrkB.mmH | | | | |
| Captured | Captured | Bound | kₐ | k_{d} | K_{D} | t_{1/2} |
| Analyte | (RU) | (RU) | (1/Ms) | (1/s) | (M) | (min) |
| H4H9780P | 59 ± 0.2 | 0 | NB | NB | NB | NB |
| H4H9814P | 48 ± 0.5 | −1 | NB | NB | NB | NB |
| H4H9816P2 | 74 ± 0.6 | 0 | NB | NB | NB | NB |
| H1M8037C* | 815.45 | 32 | 7.93E+04 | 6.94E−03 | 8.75E−08 | 1.7 |
| Comparator | | | | | | |

*indicates that mAb was captured using anti-mFc immobilized surface

### Example 4. Biacore Binding Kinetics of Surrogate Anti-TrkB Monoclonal Antibodies Binding to Different TrkB Reagents Measured at 25° C.

Equilibrium dissociation constants (K_{D} values) for TrkB binding to purified anti-TrkB monoclonal antibodies were determined using a real-time surface plasmon resonance biosensor using a Biacore T200 instrument. All binding studies were performed in 10 mM Hepes pH 7.4, 150 mM NaCl, 3 mM EDTA, and 0.05%v/v Surfactant Tween-20 (HBS-ET running buffer) at 25° C. The Biacore sensor surface was first derivatized by amine coupling with rabbit anti-mouse Fc polyclonal antibody (GE Healthcare #BR-1008-38) to capture anti-TrkB monoclonal antibodies. Binding studies were performed on the following TrkB reagents; human TrkB extracellular domain expressed with a C-terminal myc-myc-hexahistidine tag (hTrkB.mmH; SEQ ID NO: 76; NP_001018074.1), mouse TrkB extracellular domain expressed with a C-terminal myc-myc-hexahistidine tag (mTRKB.mmH; SEQ ID NO: 79; NP_001020245), and rat TrkB extracellular domain expressed with a C-terminal myc-myc-hexahistidine tag (rTRKB.mmH; SEQ ID NO: 84; XP_002721319.1). Different concentrations of TrkB reagents were first prepared in HBS-ET running buffer (90 nM-3.33 nM; 3-fold serial dilution) and were injected over anti-mouse Fc captured anti-TrkB monoclonal antibody surface for 4 minutes at a flow rate of 50 μL/minute, while the dissociation of monoclonal antibody bound TrkB reagent was monitored for 10 minutes in HBS-ET running buffer. Kinetic association (kₐ) and dissociation (k_{d}) rate constants were determined by fitting the real-time binding sensorgrams to a 1:1 binding model with mass transport limitation using Scrubber 2.0c curve-fitting software. Binding dissociation equilibrium constants (K_{D}) and dissociative half-lives (t½) were calculated from the kinetic rate constants as:

$${K}_{D} ( M ) = \frac{kd}{ka} , and {t}^{1 / 2} ( min ) = \frac{ln ( 2 )}{60 \times kd}$$

Binding kinetic parameters for hTrkB.mmH, mTrkB.mmH or rTrkB.mmH binding to different anti-TrkB monoclonal antibodies of the invention at 25° C. are shown in Tables 11 through 13.

### Results

At 25° C., the surrogate anti-TrkB monoclonal antibodies of the invention show no binding to hTrkB.mmH, as shown in Table 11.

At 25° C., the surrogate anti-TrkB monoclonal antibodies of the invention bound to mTrkB.mmH with KD values ranging from 2.39 nM to 32.4 nM, as shown in Table 12.

At 25° C., the surrogate anti-TrkB monoclonal antibodies of the invention bound to rTrkB.mmH with KD values ranging from 2.56 nM to 26.9 nM, as shown in Table 13.

TABLE 11
Binding kinetics parameters of hTrkB.mmH binding
to TrkB monoclonal antibodies at 25° C.

| | Amount of | 90 nM | | | | |
|---|---|---|---|---|---|---|
| | Analyte | hTrkB.mmH | | | | |
| Captured | Captured | Bound | kₐ | k_{d} | K_{D} | t_{1/2} |
| Analyte | (RU) | (RU) | (1/Ms) | (1/s) | (M) | (min) |
| M2aM14173N | 230 ± 0.8 | 0 | NB | NB | NB | NB |
| M2aM14178N | 125 ± 0.1 | −1 | NB | NB | NB | NB |
| M2aM14179N | 396 ± 2.1 | −2 | NB | NB | NB | NB |

TABLE 12
Binding kinetics parameters of mTrkB.mmH binding
to TrkB monoclonal antibodies at 25° C.

| | Amount of | 90 nM | | | | |
|---|---|---|---|---|---|---|
| | Analyte | mTrkB.mmH | | | | |
| Captured | Captured | Bound | kₐ | k_{d} | K_{D} | t_{1/2} |
| Analyte | (RU) | (RU) | (1/Ms) | (1/s) | (M) | (min) |
| M2aM14173N | 236 ± 1.1 | 33 | 7.41E+04 | 6.30E−04 | 8.49E−09 | 18 |
| M2aM14178N | 126 ± 0.3 | 12 | 1.03E+05 | 2.47E−04 | 2.39E−09 | 47 |
| M2aM14179N | 416 ± 4.3 | 32 | 1.15E+05 | 3.72E−03 | 3.24E−08 | 3.1 |

TABLE 13
Binding kinetics parameters of rTrkB.mmH binding
to TrkB monoclonal antibodies at 25° C.

| | Amount of | 90 nM | | | | |
|---|---|---|---|---|---|---|
| | Analyte | rTrkB.mmH | | | | |
| Captured | Captured | Bound | kₐ | k_{d} | K_{D} | t_{1/2} |
| Analyte | (RU) | (RU) | (1/Ms) | (1/s) | (M) | (min) |
| M2aM14173N | 233 ± 0.7 | 33 | 7.90E+04 | 6.32E−04 | 8.00E−09 | 18 |
| M2aM14178N | 125 ± 0.3 | 11 | 9.00E+04 | 2.30E−04 | 2.56E−09 | 50 |
| M2aM14179N | 404 ± 2.5 | 33 | 1.48E+05 | 3.98E−03 | 2.69E−08 | 2.9 |

### Example 5. Bioassay with HEK293/SRE-luc/hTrkB and HEK293/SRE-luc/mTrkB(Ecto)-hTrkB (TM-Cyto) Cells

A bioassay was developed to detect the activation of TrkB using a luciferase reporter gene under control of the serum response element (SRE) and the ligand, brain derived neurotrophic factor (BDNF, R & D Systems). HEK293 cell lines were generated that stably express a luciferase reporter (SRE response element-luciferase, SRE-luc, SA Bioscience, #CLS-010L) with either human TrkB (hTrkB, amino acids 32-429 of NP_001018074.1 or Uniprot number Q16620-1) or mouse TrkB extracellular domain fused to the transmembrane and cytoplasmic domains of human TrkB (mTrkB, amino acids 32-429 of NP_001020245.1 or Uniprot number P15209-1 fused to hTrkB, amino acids 431-822). The stable cell lines, HEK293/SRE-Luc/hTrkB and HEK293/SRE-Luc/mTrkB, were maintained in DMEM supplemented with 10% FBS, non-essential amino acids, penicillin/streptomycin/glutamine, 1 □g/mL puromycin, and 500 □g/mL G418.

For the bioassay, cells were seeded into 96-well assay plates at 20,000 cells/well in Opti-MEM™M supplemented with 0.1% FBS, penicillin/streptomycin and L-glutamine, and then incubated at 37° C. in 5% CO₂ overnight. The next morning, human BDNF or antibodies was serially diluted from 100 nM to 0.002 nM (plus a sample containing buffer alone without ligand) and added to the cells to determine the activation of TrkB signaling. The serially diluted antibodies were also tested with 100 pM of BDNF (R & D Systems, 248-BD/CF). The cells were then incubated for 5.5 hours at 37° C. in the presence of 5% CO₂. Luciferase activity was measured after the addition of OneGlo reagent (Promega) using a Victor X instrument (Perkin Elmer). The results were analyzed using nonlinear regression (4-parameter logistics) with Prism 5 software (GraphPad) to obtain EC₅₀ and IC₅₀ values. Maximum activation of antibodies was calculated using the following:

$$Max . \% Activation = \frac{( Maximum RLU achieved by antibody ) - ( RLU achieved by no BDNF )}{( Maximum RLU achieved by BDNF ) - ( RLU achieved by no BDNF )} \times 100$$

### Results Summary and Conclusions

As shown in Table 14, three anti-TrkB antibodies of the invention H4H9816P2, H4H9814P, H4H9780P showed activation of human TrkB signaling in HEK293/SRE-luc/hTrkB cells in the absence of BDNF with EC₅₀ₛ of 35-82 pM with maximum activation ranging 88-92%. Three antibodies of the invention were also tested in the presence 100 pM BDNF. Whereas 100 pM BDNF showed activation of 56% in the presence of irrelevant control mAb, Control mAb2, the antibodies of the invention showed further activation with EC₅₀ₛ of 45-76 pM with maximum activation ranging 77-80%. Three anti-TrkB antibodies of the invention did not show activation of mouse TrkB signaling in HEK293/SRE-luc/mTrkB cells in the absence of BDNF or presence of 100 pM BDNF. Control mAb 1, an anti-TrkB comparator antibody H1M8037C, showed activation of human TrkB signaling with an EC₅₀ of 76 pM with maximum activation of 78% and mouse TrkB with an EC₅₀ of 43 pM with maximum activation of 85% without BDNF. In the presence of 100 pM BDNF, Control mAb 1 activated human TrkB signaling with an EC₅₀ of 110 pM with maximum activation of 79% and mouse TrkB with an EC₅₀ of 42 pM with maximum activation of 69%, greater than the activation by Control mAb 2 with 100 pM BDNF. Control mAb 2, an irrelevant human IgG4 antibody, did not show any activation in the absence or in the presence of 100 pM BDNF.

As shown in Table 15, three anti-TrkB antibodies of the invention M2aM14173N, M2aM14178N, M2aM14179N showed activation of mouse TrkB signaling in HEK293/SRE-luc/mTrkB cells in the absence of BDNF with EC₅₀ₛ of 34-190 pM with maximum activation ranging from 76-94%. Three antibodies of the invention were also tested in the presence 100 pM BDNF. Whereas 100 pM BDNF showed activation of 60% in the presence of irrelevant isotype control mAb, Control mAb4, the antibodies of the invention showed further activation with EC50s of 17-100 pM with maximum activation ranging 67-75%. Three anti-TrkB antibodies of the invention did not show activation of human TrkB signaling in HEK293/SRE-luc/hTrkB cells in the absence of BDNF or presence of 100 pM BDNF. Control mAb 1 showed activation of human TrkB signaling with an EC₅₀ of 57 pM with maximum activation of 80% and mouse TrkB with an EC₅₀ of 43 pM with maximum activation of 85% without BDNF. In the presence of 100 pM BDNF, Control mAb 1 activated human TrkB signaling with an EC₅₀ of 110 pM with maximum activation of 79% and mouse TrkB with an EC₅₀ of 42 pM with maximum activation of 69%, greater than the activation by Control mAb 4 with 100 pM BDNF. Control mAb 3 and Control mAb 4, irrelevant mouse IgG2a isotype control antibodies, did not show any activation in the absence or in the presence of 100 pM BDNF.

### Tabulated Data Summary

TABLE 14
Activation of HEK293/SRE-Luc/hTrkB and HEK293/SRE-Luc/mTrkB cells by anti-TrkB antibodies

| | Cells | | | | | | | |
|---|---|---|---|---|---|---|---|---|
| | HEK293/SRE-luc/hTrkB | | | | HEK293/SRE-luc/mTrkB | | | |
| | BDNF | | | | | | | |
| | 1.0E−10 | | | | 7.4E−11 | | | |
| | Ligand | | | | | | | |
| | No BDNF | | 100 pM BDNF | | No BDNF | | 100 pM BDNF | |
| Antibody | EC50 (M) | Activation (%) | EC50 (M) | Activation (%) | EC50 (M) | Activation (%) | EC50 (M) | Activation (%) |
| H4H9780P | 8.2E−11 | 88 | 7.6E−11 | 77 | No | 0 | No | 55 |
| | | | | | activation | | activation | |
| H4H9814P | 3.5E−11 | 92 | 4.9E−11 | 80 | No | 0 | No | 54 |
| | | | | | activation | | activation | |
| H4H9816P2 | 6.3E−11 | 90 | 4.5E−11 | 78 | No | 0 | No | 58 |
| | | | | | activation | | activation | |
| Control | 7.6E−11 | 78 | 1.1E−10 | 79 | 4.3E−11 | 85 | 4.2E−11 | 69 |
| mAb 1 | | | | | | | | |
| (Comparator | | | | | | | | |
| H1M8037C) | | | | | | | | |
| Negative | No | 3 | No | 56 | No | 0 | No | 54 |
| Isotype | activation | | activation | | activation | | activation | |
| Control | | | | | | | | |
| mAb 2 | | | | | | | | |

TABLE 15
Activation of HEK293/SRE-Luc/hTrkB and HEK293/SRE-Luc/mTrkB cells by anti-TrkB antibodies (surrogate)

| | Cells | | | | | | | |
|---|---|---|---|---|---|---|---|---|
| | HEK293/SRE-luc/hTrkB | | | | HEK293/SRE-luc/mTrkB | | | |
| | BDNF | | | | | | | |
| | 1.3E−10 | | 1.0E−10 | | 7.4E−11 | | 1.1E−10 | |
| | Ligand | | | | | | | |
| | No BDNF | | 100 pM BDNF | | No BDNF | | 100 pM BDNF | |
| Antibody | EC50 (M) | Activation (%) | EC50 (M) | Activation (%) | EC50 (M) | Activation (%) | EC50 (M) | Activation (%) |
| M2aM14173N | No | 2 | No | 59 | 3.6E−11 | 94 | 1.7E−11 | 74 |
| | activation | | activation | | | | | |
| M2aM14178N | No | 0 | No | 52 | 1.9E−10 | 76 | 1.0E−10 | 67 |
| | activation | | activation | | | | | |
| M2aM14179N | No | 4 | No | 52 | 3.4E−11 | 87 | 2.1E−11 | 75 |
| | activation | | activation | | | | | |
| Control | 5.7E−11 | 80 | 1.1E−10 | 79 | 4.3E−11 | 85 | 4.2E−11 | 69 |
| mAb 1 | | | | | | | | |
| (Comparator | | | | | | | | |
| H1M8037C) | | | | | | | | |
| Negative | No | 0 | Not | Not | Not | Not | Not | Not |
| Isotype | activation | | tested | tested | tested | tested | tested | tested |
| Control | | | | | | | | |
| mAb 3 | | | | | | | | |
| Negative | Not | Not | No | 61 | No | 0 | No | 60 |
| Isotype | tested | tested | activation | | activation | | activation | |
| Control | | | | | | | | |
| mAb 4 | | | | | | | | |

### Example 6. In Vivo Comparison of the Effect of TrkB Agonist Antibody H4H9816P2 and IgG4 Isotype Control REGN1945 on TrkB Phosphorylation in the Brain Following Stereotaxic Injection in TrkB^{hu/hu} Mice

In order to determine the effect of a TrkB agonist antibody of the invention, H4H9816P2, on TrkB activation kinetics, a time-course study of TrkB phosphorylation following direct hippocampal injection was performed in mice homozygous for human TrkB receptor in place of mouse TrkB receptor (referred to as TrkB^{hu/hu} mice). TrkB^{hu/hu} mice (N=48) received bilateral stereotaxic injections with either 2 μL of vehicle (PBS), REGN1945 hereby noted as IgG4 isotype control antibody (27.5 mg/mL final concentration), or TrkB agonist antibody H4H9816P2 (27.5 mg/mL final concentration) into the hippocampus, −2 mm posterior and +1.5 mm lateral to bregma. In order to minimize tissue damage, injection and needle removal were both performed gradually over 5-minute intervals. TrkB^{hu/hu} mice were then sacrificed by CO₂ euthanasia approximately 30 minutes, 1 hour, 4 hours, or 18 hours post-injection. A terminal bleed was performed via cardiac puncture to collect blood, and mice were then transcardially perfused with cold heparinized saline. The brain was carefully removed from the skull, and a 2 mm³ section of tissue surrounding the injection site was dissected, collected in an Eppendorf tube and stored on ice. The brain section was then lysed in 300 uL of RIPA lysis buffer (ThermoFisher Scientific, Cat #89901) containing 2× protease and phosphatase inhibitors (ThermoFisher Scientific, Cat #78444) and stored on ice. The lysed tissue was then homogenized for further processing, aliquoted and stored at −80° C.

To assess TrkB phosphorylation in the brain tissue, immuno-precipitation and western blotting was performed. Anti-human TrkB antibody H4H10108N that does not compete for binding with H4H9816P2 was coupled to NHS-activated Sepharose beads (prepared using manufacturer's protocol; GE Healthcare, Cat #17-0906) and washed with DPBS three times to remove any residual preservation solution. Homogenized brain lysates were thawed on ice and diluted to a concentration of 1 mg/mL (brain weight to buffer volume) in a buffer composed of 1% NP-40, 0.1% Tween-20, protease and phosphatase inhibitors in TBST. The protein concentration of the homogenized brain lysate was quantified by performing a standard BCA assay per manufacturer's instructions (Thermo Scientific Pierce, Cat #23225). For every 100 ug of protein, 15 uL of anti-human TrkB antibody (H4H10108N) NHS-activated Sepharose beads were added to the brain lysate solution and the mixture was incubated overnight at 4° C. with gentle shaking at 20 rpm (Thermo rotator). The next day, samples were centrifuged at 1000× g for one minute, and the supernatant was then carefully removed. Beads were subsequently washed twice with 400 uL of Tris-buffered saline (Bio-Rad, Cat #1706435) with 1% Tween-20 (Sigma Aldrich, Cat #P9416) (TBST). After carefully aspirating the wash buffer, 60 uL of 0.1% Trifluoroacetic acid (TFA; Sigma-Aldrich, T62200) in water at pH 3.0 was added to each sample. The solution was mixed and allowed to stand for two minutes before being collected and transferred into a separate tube. This process was repeated with another 60 uL of 0.1% TFA at pH 3.0. The two 0.1% TFA solutions for each sample were then combined, and 2 uL of 1M Tris-HCl (ThermoFisher Scientific, Cat #15567-027), at pH 8.5, was added.

The solution was dried using a speed vacuum and then re-suspended and reduced with a mixtue of 20 uL of 1× Laemmli Buffer (Bio-Rad, Cat #1610737) plus 355 nM 2-mercaptoethanol (BME; Gibco, Cat #21985-023). Samples were boiled at 95° C. for 10 minutes and loaded onto a 10-well, Mini-Protean 4-15% Tris-Glycine gel (Bio-Rad, Cat #4561086). After electrophoresis, protein samples were transferred from the Tris-Glycine gel onto a PVDF membrane (Bio-Rad, Cat #170-4156) via the Trans-Blot Turbo Transfer System (Bio-Rad, Cat #1704156) over the course of 30 minutes at a constant rate of 1.3 A and 25 V. After the transfer, the membrane was blocked with 2.5% milk (Bio-Rad, Cat #170-6406) in TBST for one hour at room temperature, and subsequently probed overnight with either an anti-phospho-TrkB antibody (Novus, Cat #NB100-92656) diluted 1:1000 in a solution of 2.5% BSA or anti-TrkB primary antibody (Cell Signaling, Cat #4603) diluted to 1:1000 in 2.5% milk TBST at 4° C. on a shaker at 30 rpm. The next day, blots were washed with TBST and incubated with an anti-rabbit IgG antibody conjugated with horseradish peroxidase (Jackson, Cat #111-035-144) at 1:1000 in 1% milk in TBST for 1 hour at room temperature. Blots were then washed again, developed with ECL solution (PerkinElmer, Inc. Cat #RPN2106), and subsequent image exposures were taken every 30 seconds.

### Results Summary and Conclusions

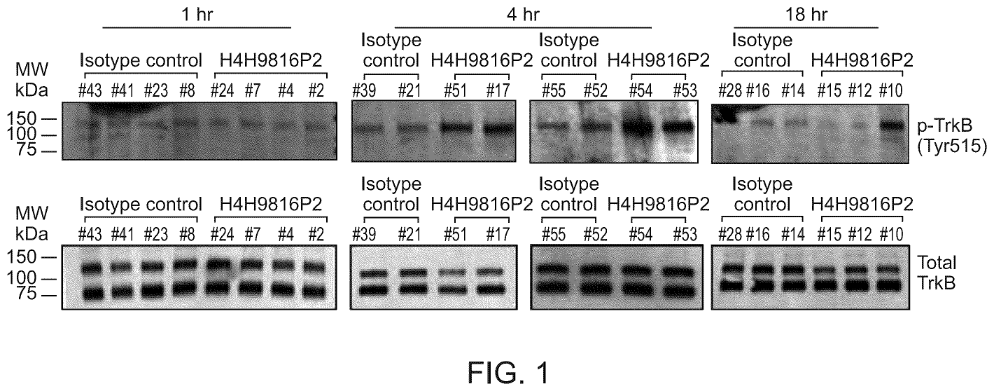

Immunoprecipitation and subsequent western blotting of protein derived from TrkB^{hu/hu} mouse brain lysates demonstrated that hippocampal TrkB phosphorylation was detectable in mice injected with a TrkB agonist antibody, H4H9816P2, but not in mice treated with vehicle or isotype control antibody, as shown FIG. 1. Amongst the timepoints assessed, TrkB phosphorylation peaked at 4 hours after stereotaxic injection in mice dosed with H4H9816P2. TrkB phosphorylation was also detected by western blot at 18 hours post-dosing in some, but not all mice. Conversely, injection of vehicle and IgG4 isotype control antibody did not induce TrkB phosphorylation at any timepoint. Western blotting also indicated that the total TrkB receptor levels were downregulated in some, but not all TrkB^{hu/hu} mice dosed with H4H9816P2 relative to vehicle and isotype control treated mice. Total TrkB levels appeared to be slightly downregulated in H4H9816P2-treated subjects at 18 hours post-dosing. Thus, these results indicate that direct injection of the TrkB agonist antibody, H4H9816P2, induces phosphorylation of hippocampal TrkB receptors in TrkB^{hu/hu} mice.

### Example 7. In Vivo Comparison of the Effect of H4H9816 and Isotype Control REGN1945 Antibodies on Body Weight and Metabolism in TrkB^{hu/hu} Mice

To determine the effect of a TrkB agonist antibody of the invention, H4H9816P2, on body weight and composition, a metabolic study of mice homozygous for the expression of human TrkB receptor in place of the mouse TrkB receptor (TrkB^{hu/hu} mice) was conducted following a single sub-cutaneous antibody injection. TrkB^{hu/hu} mice (male, 20 weeks old) were first transferred from group-cage to single-cage housing for two weeks of acclimatization. After this period, mice were transferred to metabolic cages (CLAMS, Columbus Instruments) to assess changes in food and water consumption, locomotion, energy expenditure and respiration following antibody administration. Regular powdered chow was stored in a floor chamber on a spring-loaded scale (Mettler Toledo, PL602E) to measure food consumption via changes in total chow weight. Water was accessible via a cage-top spout and intake was measured by tracking changes in pump-line volume (Oxymax®/CLAMS Liquid Unit). CLAMS metabolic cages measured each of these parameters in continuous, 16-18 minute intervals throughout the duration of the study. Metabolic data was analyzed in single measures and summarized in 24-hour intervals containing one complete dark and light cycle using OXYMAX®/CLAMS software (Columbus instruments, v5.35). After acclimating to the cages for two weeks, TrkB^{hu/hu} mice received a single 50 mg/kg sub-cutaneous dose of either a TrkB agonist antibody, H4H9816P2, or an IgG4 isotype control antibody in PBS at pH7.2. A group of naïve control TrkB^{hu/hu} mice did not receive an injection. Mice were weighed immediately prior to dosing, and at 24, 48, 72, 96, and 120 hours post-dosing. In order to measure each mouse's body composition, Nuclear Magnetic Resonance Relaxometry, also referred to as Quantitative Magnetic Resonance, was performed using a EchoMRI™-500 Analyzer (EchoMRI LLC). Prior to dosing, mice were placed in a clear plastic holder and inserted into the NMR-MRI device to measure each subject's lean mass, fat mass, and hydration status. Measurements were performed over the course of 0.5-3.2 minutes per mouse, and were taken again approximately 120 hours after dosing.

### Results Summary and Conclusions

Daily body weight monitoring was performed to determine whether a single subcutaneous injection of H4H9816P2 induces weight loss in TrkB^{hu/hu} mice. Prior to dosing, there were no significant differences in the average body weight of the three treatment groups, as each had an average pre-dose body weight of 28.39-29.85 g (Table 16). At 48 hours post-dosing, however, H4H9816P2-treated TrkB^{hu/hu} mice lost an average of 1.70 g, or 5.96% of their pre-dose body weight. At the same time point, naïve and isotype control antibody-treated TrkB^{hu/hu} mice gained between 1.79-2.37% of their pre-dose body weight. H4H9816P2-treated TrkB^{hu/hu} mice continued to lose weight throughout the full time course of the study, and by 72 and 96 hours post-dosing these mice had lost an average of 8.42% and 11.80% of their pre-dose body weight, respectively. At 120 hours post-dosing, H4H9816P2-treated TrkB^{hu/hu} mice had lost an average of 12.67% of their pre-dose body weight. Conversely, naïve and isotype control-treated TrkB^{hu/hu} mice did not exhibit any loss in pre-dose body weight throughout the study. As body weight in H4H9816P2-treated TrkB^{hu/hu} mice was significantly reduced relative to both naïve and isotype controls at 48, 72, 96, and 120 hours post-dosing, it was determined that TrkB agonist antibody H4H9816P2 induced significant body weight loss in TrkB^{hu/hu} mice.

TABLE 16
Body weight of TrkB^{hu/hu} mice after dosing
with TrkB agonist antibody H4H9816P2

| | | Mean | Mean | Mean | Mean | Mean |
|---|---|---|---|---|---|---|
| | Mean | body | body | body | body | body |
| | pre-dose | weight | weight | weight | weight | weight |
| | body | (g) 24 | (g) 48 | (g) 72 | (g) 96 | (g) 120 |
| | weight | hours | hours | hours | hours | hours |
| | (g) | post-dose | post-dose | post-dose | post-dose | post-dose |
| | (±SD) | (±SD) | (±SD) | (±SD) | (±SD) | (±SD) |
| | Percent | Percent | Percent | Percent | Percent | Percent |
| | change | change | change | change | change | change |
| | from | from | from | from | from | from |
| | pre-dose | pre-dose | pre-dose | pre-dose | pre-dose | pre-dose |
| | body | body | body | body | body | body |
| Experimental | weight | weight | weight | weight | weight | weight |
| group | (+/−SD) | (+/−SD) | (+/−SD) | (+/−SD) | (+/−SD) | (+/−SD) |
| Naive | 28.85 | 29.69 | 29.36 | 29.32 | 29.29 | 28.88 |
| (n = 3) | (+/−0.81) | (+/−0.97) | (+/−1.10) | (+/−1.29) | (+/−1.10) | (+/−1.04) |
| | N/A | +2.91% | +1.79% | +1.65% | +1.54% | +0.10% |
| | | (+/−0.62) | (+/−1.62) | (+/−2.24) | (+/−1.22) | (+/−1.05) |
| Isotype control | 29.21 | 30.27 | 29.90 | 30.08 | 29.87 | 29.69 |
| (n = 4) | (+/−2.68) | (+/−2.51) | (+/−2.63) | (+/−2.69) | (+/−2.52) | (+/−2.68) |
| | N/A | +3.61% | +2.37% | +2.98% | +2.25% | +1.65% |
| | | (+/−1.68) | (+/−1.50) | (+/−1.09) | (+/−1.56) | (+/−0.81) |
| H4H9816P2 | 28.39 | 27.87 | 26.69 | 26.00* | 25.04** | 24.79** |
| (n = 4) | (+/−1.35) | (+/−1.29) | (+/−0.87) | (+/−0.98) | (+/−1.03) | (+/−1.36) |
| | N/A | −1.83% | −5.96% | −8.42% | −11.80% | −12.67% |
| | | (+/−0.56) | (+/−1.88) | (+/−1.85) | (+/−1.52) | (+/−1.66) |

Note:
Statistical significance determined by two-way ANOVA with Tukey's multiple comparison post-hoc test is indicated
(*= p < 0.05,
**= p < 0.01,
***= p < 0.001,
****= p < 0.0001, compared to isotype control group: TrkB^{hu/hu} mice dosed with 50 mg/kg isotype control antibody.

The effect of TrkB agonist antibody H4H9816P2 injection on body composition was also measured by performing NMR-MRI on each subject before and after dosing. Prior to dosing, the three treatment groups of TrkB^{hu/hu} mice did not exhibit any significant differences in fat mass or lean mass, as each group had an average of 4.19-4.75 g of fat mass and 21.32-21.70 g of lean mass (Table 17). Following antibody administration, however, TrkB^{hu/hu} mice dosed with H4H9816P2 lost an average of 48.90% of their total body fat mass over the course of the study (Table 17). Naïve and isotype control antibody-treated TrkB^{hu/hu} mice lost an average of 8.49% and 9.48% of their pre-dose fat mass, respectively, which was significantly less than H4H9816P2-treated subjects (Table 17). Furthermore, H4H9816P2-treated TrkB^{hu/hu} mice lost an average of 7.84% of their lean mass throughout the study, which was significantly greater than the 2.41% and 1.75% of average pre-dose lean mass lost by naïve and isotype control antibody-treated groups, respectively (Table 17). As such, the described body weight loss could be explained by a significant loss of fat mass and a modest loss of lean mass following injection of TrkB agonist antibody H4H9816P2 in TrkB^{hu/hu} mice.

TABLE 17
Body composition of TrkB^{hu/hu} mice after dosing
with TrkB agonist antibody H4H9816P2

| | | | Mean | | | Mean |
|---|---|---|---|---|---|---|
| | | Mean | fat | | Mean | lean |
| | Mean | fat | mass | Mean | lean | mass |
| | pre-dose | mass (%) | change (%) | pre-dose | mass (%) | change (%) |
| | fat | 120 hours | 120 hours | lean | 120 hours | 120 hours |
| Experimental | mass (%) | post-dose | post-dose | mass (%) | post-dose | post-dose |
| group | (±SD) | (±SD) | (±SD) | (±SD) | (±SD) | (±SD) |
| Naive | 4.65 | 4.27 | −8.49 | 21.45 | 20.94 | −2.41 |
| (n = 3) | (+/−0.32) | (+/−0.55) | (+/−7.18) | (+/−0.79) | (+/−0.98) | (+/−1.81) |
| Isotype control | 4.75 | 4.40 | −9.48 | 21.70 | 21.32 | −1.75 |
| (n = 4) | (+/−2.98) | (+/−2.98) | (+/−6.00) | (+/−0.50) | (+/−0.35) | (+/−0.98) |
| H4H9816P2 | 4.19 | 2.14 | −48.90**** | 21.32 | 19.64 | −7.84*** |
| (n = 4) | (+/−1.15) | (+/−0.64) | (+/−5.06) | (+/−1.87) | (+/−1.69) | (+/−0.94) |

Note:
Statistical significance determined by Kruskal-Wallis One-way ANOVA with Tukey's multiple comparison post-hoc test is indicated
(*= p < 0.05,
**= p < 0.01,
***= p < 0.001,
****= p < 0.0001, compared to isotype control group: TrkB^{hu/hu} mice dosed with 50 mg/kg isotype control antibody.

In addition to assessing the effects of TrkB agonist antibody H4H9816P2 injection on body weight and composition in TrkB^{hu/hu} mice, feeding, drinking, and locomotor activity were continuously measured by metabolic cages. Prior to dosing, TrkB^{hu/hu} mice consumed an average of 3.49-3.73 g of chow per day. Within 24 hours of dosing, however, H4H9816P2-treated TrkB^{hu/hu} mice significantly reduced their food intake to 2.20 g of chow per day. The average level of food intake in H4H9816P2-treated TrkB^{hu/hu} mice did not exceed 2.49 g of chow per day throughout the remainder of the study, while naïve and isotype antibody-treated TrkB^{hu/hu} mice consistently consumed an average of 3.62-4.07 g of chow per day (Table 18).

Similarly, there were no significant differences in daily water consumption between treatment groups prior to dosing. TrkB^{hu/hu} mice consumed an average of 4.67-5.55 mL of water per day in each treatment group (Table 19). After dosing, H4H9816P2-treated TrkB^{hu/hu} mice reduced their water intake to 2.05-3.24 mL of water per day. This was significantly lower than naïve and isotype control antibody-treated TrkB^{hu/hu} mice, which consistently consumed 4.50-5.77 mL of water per day throughout the study (Table 19). Thus, injection of the TrkB agonist antibody, H4H9816P2, appeared to result in a significant reduction of both food and water intake in TrkB^{hu/hu} mice relative to both naïve and isotype controls.

TABLE 18
Food consumption of TrkB^{hu/hu} mice after dosing with TrkB agonist antibody H4H9816P2

| | Mean total | Mean total | Mean total | Mean total | Mean total |
|---|---|---|---|---|---|
| | food intake | food intake | food intake | food intake | food intake |
| | (g) 0-24 | (g) 0-24 | (g) 24-48 | (g) 48-72 | (g) 72-96 |
| Experimental | hours pre- | hours post- | hours post- | hours post- | hours post- |
| group | dose (±SD) | dose (±SD) | dose (±SD) | dose (±SD) | dose (±SD) |
| Naive (n = 3) | 3.51 | 3.98 | 3.76 | 3.62 | 3.91 |
| | (+/−0.53) | (+/−0.08) | (+/−0.19) | (+/−0.35) | (+/−0.18) |
| Isotype control | 3.73 | 4.07 | 3.99 | 3.89 | 3.80 |
| (n = 4) | (+/−0.48) | (+/−0.23) | (+/−0.17) | (+/−0.22) | (+/−0.22) |
| H4H9816P2 | 3.49 | 2.20**** | 2.08**** | 2.18**** | 2.49*** |
| (n = 4) | (+/−1.07) | (+/−0.16) | (+/−0.36) | (+/−0.37) | (+/−0.47) |

Note:
Statistical significance determined by Kruskal-Wallis One-way ANOVA with Tukey's multiple comparison post-hoc test is indicated (* = p < 0.05, ** = p < 0.01, ***= p < 0.001, ****= p < 0.0001, compared to isotype control group: TrkB^{hu/hu} mice dosed with 50 mg/kg isotype control antibody.

TABLE 19
Water consumption of TrkB^{hu/hu} mice after dosing with TrkB agonist antibody H4H9816P2

| | Mean total | Mean total | Mean total | Mean total | Mean total |
|---|---|---|---|---|---|
| | water intake | water intake | water intake | water intake | water intake |
| | (mL) 0-24 | (mL) 0-24 | (mL) 24-48 | (mL) 48-72 | (mL) 72-96 |
| Experimental | hours pre- | hours post- | hours post- | hours post- | hours post- |
| group | dose (±SD) | dose (±SD) | dose (±SD) | dose (±SD) | dose (±SD) |
| Naive (n = 3) | 4.79 (+/−0.21) | 5.42 (+/−0.94) | 4.96 (+/−0.91) | 4.57 (+/−0.56) | 4.88 (+/−0.32) |
| Isotype control (n = 4) | 5.55 (+/−1.23) | 4.50 (+/−1.08) | 5.08 (+/−1.39) | 5.09 (+/−1.10) | 5.77 (+/−1.62) |
| H4H9816P2 (n = 4) | 4.67 (+/−1.13) | 2.25** (+/−0.55) | 3.24* (+/−1.10) | 2.05*** (+/−0.29) | 2.25**** (+/−0.24) |

Note:
Statistical significance determined by Kruskal-Wallis One-way ANOVA with Tukey's multiple comparison post-hoc test is indicated (*= p < 0.05, **= p < 0.01, ***= p < 0.001, ****= p < 0.0001, compared to isotype control group: TrkB^{hu/hu} mice dosed with 50 mg/kg isotype control antibody.

To determine the effects of antibody treatment on activity, locomotion was analyzed by OXYMAX®/CLAMS software (Columbus instruments, v5.35), which continuously measured the total number of x-plane ambulations of each mouse. One mouse exhibited hyperactivity prior to dosing and was removed from post-dose statistical analysis. While naïve and isotype antibody-treated subjects consistently registered an average of 11,000-15,000 ambulations per day throughout the study, H4H9816P2-treated TrkB^{hu/hu} registered 28,260 ambulations between 24-48 hours post-dosing, and registered 21,193 and 27,028 ambulations from 48-72 and 72-96 hours post-dosing, respectively (Table 20). H4H9816P2-treated TrkB^{hu/hu} mice registered more total ambulation counts at each time point following antibody administration, suggesting hyperactivity to be an additional effect of H4H9816P2 injection. In combination, these effects suggest that a single subcutaneous injection of the TrkB agonist antibody, H4H9816P2, induced significant changes in body weight, body composition, metabolism, and locomotion in TrkB^{hu/hu} mice.

TABLE 20
Locomotion of TrkB^{hu/hu} mice after dosing with TrkB agonist antibody H4H9816P2

| | Mean total | Mean total | Mean total | Mean total | Mean total |
|---|---|---|---|---|---|
| | ambulations | ambulations | ambulations | ambulations | ambulations |
| | (counts) 0-24 | (counts) 0-24 | (counts) 24-48 | (counts) 48-72 | (counts) 72-96 |
| Experimental | hours pre- | hours post- | hours post- | hours post- | hours post- |
| group | dose (±SD) | dose (±SD) | dose (±SD) | dose (±SD) | dose (±SD) |
| Naive (n = 3) | 16562 (+/−3380) | 14692 (+/−2792) | 14387 (+/−6126) | 13279 (+/−3607) | 12525 (+/−4121) |
| Isotype Control | 18105 (+/−4085) | 13380 (+/−2730) | 13049 (+/−3376) | 11371 (+/−2552) | 11468 (+/−2088) |
| (REGN1945) (n = 4) | | | | | |
| H4H9816P2 (n = 4) | 13292 (+/−5294) | 16575 (+/−6836) | 28260 (+/−19874) | 21193 (+/−6668) | 27028* (+/−10969) |

Note:
Statistical significance determined by Kruskal-Wallis One-way ANOVA with Tukey's multiple comparison post-hoc test is indicated (*= p < 0.05, **= p < 0.01, ***= p < 0.001, ****= p < 0.0001, compared to isotype control group: TrkB^{hu/hu} mice dosed with 50 mg/kg isotype control antibody.

### Example 8. Optic Nerve Transection Model to Determine the Effect of Anti-TrkB Antibody on Retinal Ganglion Cell (RGC) Survival

All procedures were conducted in accordance with the ARVO Statement for Use of Animals in Ophthalmic and Vision Research and the Regeneron Pharmaceutical Inc. IACUC. Adult female TrkB humanized rats (Velocigene, Regeneron Pharmaceutical Inc.), 8-10 weeks old, each weighing 200-250 g, were used. All surgical procedures on rats were performed under general anesthesia using an intraperitoneal injection of ketamine (63 mg/kg) and xylazine (6.0 mg/kg). Eye ointment containing erythromycin (0.5%, Bausch & Lomb) was applied to protect the cornea.

### Intraorbital Optic Nerve Axotomy and Intravitreal Injection

The left optic nerve (ON) was exposed intraorbitally, its dura was opened. ON was transected about 1.5 mm behind the globe. Care was taken to avoid damaging the blood supply to the retina. Intravitreal injections were performed just posterior to the pars plana with a pulled glass pipette connected to a 50 μl Hamilton syringe. Care was taken not to damage the lens. Rats with any significant postoperative complications (e.g., retinal ischemia, cataract) were excluded from further analysis. Animals were allocated to different experimental groups. One control group received intravitreal injections of 3μl isotype control REGN1945 (46.6 μg/μl); the other group received injection of 3 μl anti-human TrkB antibody H4H9816P2 (45.7 μg/μl) at 3 and 10 days after ON axotomy.

In another experiment, a dose-response of anti-human TrkB antibody H4H9816P2 was tested. 1-9 months old homozygous TrkB humanized rats received intravitreal injection of 3 ul anti-human TrkB antibody H4H9816P2 (0.01, 0.1, 1 or 10 μg/μl) or isotype control REGN1945 (10 μg/μl) at 3 and 10 days after ON axotomy.

### Immunohistochemical Staining and Counting of Viable RGCs

Brn3a (brain-specific homeobox/POU domain protein 3A) was used as a marker for surviving retinal ganglion cells (RGCs), because it has been shown to be an efficient and reliable method for selective labelling of viable RGCs in retinal whole mounts after ON injury (Nadal-Nicolás F M, Jiménez-López M, Sobrado-Calvo P, Nicto-López L, Cánovas-Martínez I, Salinas-Navarro M, Vidal-Sanz M, Agudo M., Invest Ophthalmol Vis Sci. 2009 August;50(8):3860-8). To immunostain for Brn3a, retinas were blocked in 10% normal donkey serum and 0.5% Triton X-100 for 1 hr, then incubated in the same medium with Brn3a antibody (1:400; Cat #: sc-31984, Santa Cruz) 2 hr at room temperature. After further washes retinas were incubated with Alexa594-conjugated donkey anti-goat secondary antibody (1:400; Cat #: A-11058, Invitrogen) overnight at 4° C.

### Results Summary and Conclusions

To assess the effect of TrkB agonist antibody on RGC survival in vivo, we used a complete optic nerve transection model. TrkB agonist antibody (H4H9816P2) or isotype (negative control) antibody was applied at 3 and 10 days after surgery. Animals were euthanized 14 days after axotomy. The RGC density in the uninjured contralateral eye is similar in the three TrkB genotypes, average around 1600 per mm² as shown in Table 21. The density of surviving RGCs was assessed in retinal whole mounts using Brn3a staining. It was observed that in homozygous TrkB humanized rat, TrkB agonist antibody (H4H9816P2) significantly (p<0.01, Mann-Whitney test) increased RGC survival compared with controls (685±106 vs. 255±66 RGCs per mm²). In the heterozygous TrkB humanized rat, there is also significant (p<0.05, Mann-Whitney test) survival effect of TrkB agonist antibody (444±90 vs. 208±50 RGCs per mm²). In the wild type TrkB rat, there is slight but not significant increase of RGC number in TrkB agonist antibody compared to isotype control (Table 22). In the dose-response experiment, the RGC density was quantified in retinal whole mounts using Brn3a staining 14 days after axotomy. There are clear dose-response of TrkB agonist antibody. Compared to the antibody control group (168±43 RGCs per mm²), TrkB agonist antibody (H4H9816P2) significantly (p<0.01, one-way ANOVA with Tukey post test) increased RGC survival in 3 ug (564+/−124 RGCs per mm²) or 30 ug (543+/−242 RGCs per mm²) per injection group. There is no difference between 3 and 30 ug groups. In groups that received 0.03 ug (202+/−96 RGCs per mm²) or 0.3 ug (337+/−210 RGCs per mm²) per injection group, there is only a trend but not significant increase in RGC survival (Table 23). In conclusion, the TrkB agonist antibody H4H9816P2 showed significantly increased RGC survival in TrkB^{hu/hu} and TrkB^{hu/+} rats. Conclusion:

TrkB agonist Ab (H4H9816P2) dose dependently significantly increased RGC survival in a humanized TrkB rat.

TABLE 21
RGC quantification (RGCs/mm²) in uninjured control eye
TRKB genotypes

| hu/hu | hu/+ | +/+ |
|---|---|---|
| 1637.3 | 1720.4 | 1636.3 |
| 1551.5 | 2064.6 | 1670.2 |
| 1651.4 | 1738.8 | 1873.4 |
| 1628.2 | 2029.8 | 1725.4 |
| 1804.7 | 1929.6 | 1973.4 |
| 1741.3 | 1645.9 | |
| 1739.7 | 1761.5 | |
| 1698.8 | 1787.5 | |
| 1862.5 | 1914.0 | |
| 1779.4 | | |

TABLE 22
RGC quantification (RGCs/mm²) after optic nerve injury

| | H4H9816P2 | | | | | Isotype control Ab | | | | |
|---|---|---|---|---|---|---|---|---|---|---|
| | A:Y1 | A:Y2 | A:Y3 | A:Y4 | A:Y5 | B:Y1 | B:Y2 | B:Y3 | B:Y4 | B:Y5 |
| Hu/Hu | 790.1 | 737.1 | 756.3 | 587.8 | 555.7 | 322.8 | 295.0 | 286.9 | 171.3 | 197.9 |
| Hu/+ | 530.4 | 457.5 | 522.9 | 390.6 | 319.2 | 231.0 | 184.6 | 265.1 | 151.3 | |
| +/+ | 320.9 | 355.5 | 256.9 | 342.7 | | 112.3 | | | | |

TABLE 23
RGC quantification (RGCs/mm²) in dose-response study

| Control Ab | H4H9816P2 | | | |
|---|---|---|---|---|
| 30 ug/ivt | 0.03 ug/ivt | 0.3 ug/ivt | 3 ug/ivt | 30 ug/ivt |
| 222.3 | 162.7 | 341.7 | 637.9 | 493.0 |
| 205.9 | 190.2 | 269.1 | 686.4 | 613.5 |
| 131.7 | 127.7 | 292.2 | 533.2 | 557.6 |
| 136.3 | 174.0 | 252.8 | 574.7 | 227.1 |
| 144.3 | 163.8 | 128.3 | 334.4 | 954.3 |
| | 392.6 | 740.9 | 620.6 | 411.3 |

### Example 9. Effect of anti-TrkB Antibodies on the Akt and Erk Signaling Pathways

All procedures were conducted in accordance with the ARVO Statement for Use of Animals in Ophthalmic and Vision Research and the Regeneron Pharmaceutical Inc. IACUC. Primary mouse cortical neurons were isolated and cultured from humanized TrkB mice (MAID 7139) (Nat Protoc. 2012 September;7(9):1741-54. doi: 10.1038/nprot.2012.099). A Western Blot (WB) was performed to determine the effects of TrkB agonist Ab on the downstream pathways of Akt and Erk (p-Akt, p-Erk1/2). Primary cortical neuron from postnatal day 1 (P1) humanized TrkB mouse pups were cultured for 4 days (DIV-4) in NeuralQ Basal Medium (Global Stem, cat. #GSM-9420) supplemented with GS21 Neural Supplement (Global Stem, cat. #GSM-3100), Glutamax (Invitrogen, cat. #35050-061) and Penicilin/Streptomycin. Cells were treated with TrkB agonist Abs: H4H9816P-L1 (10 ug/ml), H4H9780P-L1 (10 ug/ml), H4H9814P-L1 (10 ug/ml), IgG4 isotype control REGN1945 (10 ug/ml), control antibody H1M8037C-L1 (10 ug/ml), BDNF (lug/ml), for 15 minutes or 2 hours. Western Blot was performed to determine if the agonists have a difference in downstream signaling maintenance and strength. Treated cells were rinsed and scraped in cold PBS containing 1% protease and phosphatase inhibitors (Sigma). Protein concentration was determined by Bradford protein assay (Pierce). Samples (50 μg) were separated by SDS-PAGE in 3-8% Tris-Acetate reduced gels (Novex) and transferred to a nitrocellulose membrane (Bio-Rad).

The membrane was incubated for 1 hour in blocking solution containing 5% milk and 0.1% Tween-20, pH 7.6. This was followed by overnight incubation at 4° C. in the blocking buffer containing 5% BSA, 0.1% Tween-20 and rabbit anti-phosphoTrk (Cell Signaling, cat. #9141, 1:500), rabbit anti-phospho-Akt (Cell Signaling, cat. #9271, 1:1000) or rabbit anti-phospho-ERK1/2 antibody (Sigma, cat. #E7028, 1:5000). Subsequently, the labeled proteins were visualized by incubation with a horseradish peroxidase (HRP) conjugated anti-goat, mouse or rabbit IgG followed by development with a chemiluminescence substrate for HRP (Pierce). To determine the amounts of total TrkB, MAPK or Akt present in each lane, the nitrocellulose membranes were stripped of the antibodies in stripping buffer (Pierce) for 20 min and incubated with rabbit anti-TrkB (Cell Signaling, cat. #4603, 1:1000), rabbit anti-Erk1/2 (Cell Signaling, cat. #06-182, 1:1000) or rabbit anti-Akt antibody (Cell Signaling, cat. #9272, 1:1000) and then visualized as described above. Beta-Actin (Sigma, cat. #A5316, 1:20000 and GAPDH (Sigma, cat. #G9295) were probed as sample loading control.

### Results Summary and Conclusions

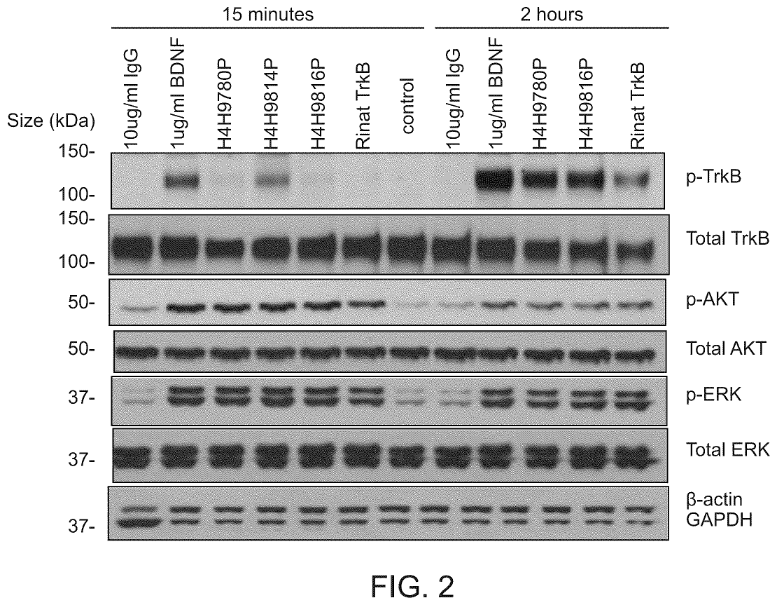

As shown in FIG. 2, at 15 mins after the incubation, while all the TrkB agonist Ab showed activation of MAPK/ERK and PI3K/Akt pathway, only BDNF and H4H9814P showed TrkB phosphorylation. 2 hrs after incubation all the TrkB agonist Abs showed activation of TrkB.

### Example 10. Effect of Agonist Anti-TrkB Antibodies on Survival of SH-SY5Y Cells Human Neuroblastoma SH-SY5Y Cell Line in Vitro Culture

Neuroblastoma cell line SH-SY5Y (Sigma ATCC #94030304, cat. #11C016) cells were plated in growth media containing DMEM:F12 (Invitrogen cat #11330), Pen/Strep (Invitrogen cat. #15140), FBS 10% (Invitrogen cat #10082-147) at 37C in 5% CO2. At passage 23-27 cells were seeded into 96 well plates in differentiation media containing all-trans 10 uM Retinoic Acid (Alfa Aesar cat. #44540), DMEM:F12 (Invitrogen cat #11330), Pen/Strep (Invitrogen cat #15140), FBS 10% (Invitrogen cat #10082-147). Cells (30K/well) were differentiated for 4 days. Antibodies were screened in survival bioassay in which culture was changed to serum free differentiation media (100 ul/well) containing different doses of antibodies (100-0.01 ug/ml). After 2 days CCK8 (Dojindo, cat. #CK04) reagent was added (10 ul/well), plates were incubated for 3-4 hours, OD was measured at 450 nm (Victor or FlexStation III) to determine the percentage of surviving cells. Data was normalized to the serum free media without treatment. Serum free treatment without antibodies=100% survival

### Results Summary and Conclusions

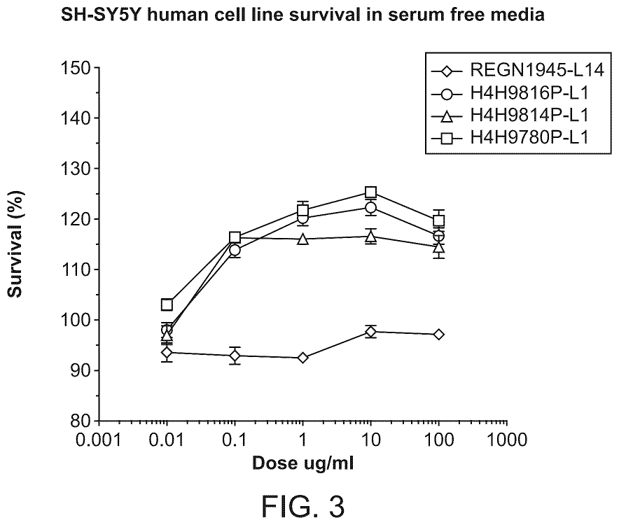

As shown in FIG. 3 and Table 23, all of the agonist TrkB antibodies of the invention showed a significant dose-dependent increase in the survival of SH-SY5Y cells as compared to the negative isotype control antibody (p<0.0001 by two way ANOVA).

TABLE 24
Average % survival

| | | | | REGN1945- |
|---|---|---|---|---|
| Dose | H4H9816P- | H4H9780P- | H4H9814P- | L14 (negative |
| ug/ml | L1 | L1 | L1 | isotype control) |
| 100 | 116.7 | 119.7 | 114.5 | 97.1 |
| 10 | 122.3 | 125.3 | 116.6 | 97.7 |
| 1 | 120.2 | 121.7 | 116.0 | 92.5 |
| 0.1 | 113.9 | 116.4 | 116.3 | 92.9 |
| 0.01 | 98.0 | 103.0 | 97.0 | 93.6 |

### Example 11. Pharmacokinetic Assessment of an Anti-TrkB Antibody in Humanized TrkB and WT Mice

Evaluation of the pharmacokinetics of an anti-TrkB antibody, H4H9816P2 was conducted in humanized TrkB mice (mice homozygous for human TrkB expression, TrkB^{hu/hu} ) and WildType (WT) mice. Cohorts contained 5 mice per mouse strain. All mice received a single sub-cutaneous (SC) 10 mg/kg dose. Blood samples were collected at 6 hours and 1, 2, 3, 6, 9, 16, 21 and 30 days post dosing. Blood was processed into serum and frozen at −80° C. until analyzed.

Circulating antibody concentrations were determined by total human IgG4/hIgG1 antibody analysis using the GyroLab xPlore™ (Gyros, Uppsala, Sweden). Briefly, biotinylated mouse anti-human IgG4/IgG1-specific monoclonal antibody (REGN2567) diluted to 100 μg/mL in antibody dilution buffer (0.05% Tween-20+PBS) was captured on a Gyrolab Bioaffy 200 CD, which contained affinity columns preloaded with streptavidin-coated beads (Dynospheres™). The standard used for calibration in this assay was H4H9816P at concentrations ranging from 0.488 to 2000 ng/mL in dilution buffer (0.5% BSA+PBS) containing 0.1% normal mouse serum (NMS). Serum samples were diluted 1:100 in the antibody dilution buffer. Human IgG captured on the anti-REGN2567-coated affinity columns on the CD, run at room temperature, was detected by addition of 0.5 μg/mL Alexa-647-conjugated mouse anti-human kappa monoclonal antibody (REGN654) diluted in detection buffer (Rexxip F buffer); and the resultant fluorescent signal was recorded in response units (RU) by the GyroLab xPlore instrument. Sample concentrations were determined by interpolation from a standard curve that was fit using a 5-parameter logistic curve fit using the Gyrolab Evaluator Software. Average concentrations from 2 replicate experiments were used for subsequent PK analysis.

PK parameters were determined by non-compartmental analysis (NCA) using Phoenix®WinNonlin® software Version 6.3 (Certara, L.P., Princeton, NJ) and an extravascular dosing model. Using the respective mean concentration values for each antibody, all PK parameters including observed maximum concentration in serum (Cₘₐₓ), estimated half-life observed (t½), and area under the concentration curve versus time up to the last measureable concentration (AUCₗₐₛₜ) were determined using a linear trapezoidal rule with linear interpolation and uniform weighting.

### Results Summary and Conclusions

Following 10 mg/kg s.c. administration of anti-TrkB antibody, H4H9816P2, similar maximum concentrations (Cₘₐₓ) of antibody were observed by day 1 or 2 in both TrkB^{hu/hu} and WT mice (135 and 131 μg/mL, respectively, see Table 26). By day 9, H4H9816P2 exhibited steeper drug elimination in TrkB^{hu/hu} mice than in WT mice, indicating a target-mediated effect. Day 30 antibody concentrations were about 35-fold less in TrkB^{hu/hu} mice. Antibody exposure (AUCₗₐₛₜ) for H4H9816P2 in WT mice was ˜1.7-fold higher than seen in TrkB^{hu/hu} mice (1730 and 1020 d*μg/mL, respectively). WT mice also exhibited about a 3-fold increase in half-life (T1/2) over TrkB^{hu/hu} mice (8.4 and 2.9 days, respectively).

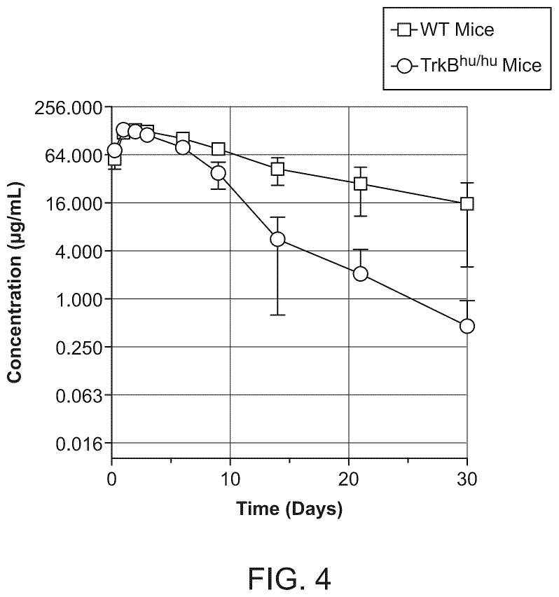

A summary of the data for total anti-TrkB antibody concentrations are summarized in Table 25, mean PK parameters are described in Table 26 and mean total antibody concentrations versus time are shown in FIG. 4.

TABLE 25
Mean Concentrations (±SD) of Total IgG in Serum
Following a Single 10 mg/kg Sub-Cutaneous Injection of
H4H9816P2 in TrkB^{hu/hu} and WildType Mice Over Time

| | | Total mAb Concentration | |
|---|---|---|---|
| | | in Mouse Serum | |
| | Time | 10 mg/kg | |
| Antibody | (d) | Mean (μg/mL) | +/−SD |
| TrkB^{hu/hu} Mice | 0.25 | 72.42 | 4.06 |
| | 1 | 132.0 | 18.0 |
| | 2 | 124.9 | 15.9 |
| | 3 | 113.4 | 11.8 |
| | 6 | 78.72 | 9.98 |
| | 9 | 37.74 | 14.0 |
| | 16 | 5.592 | 4.97 |
| | 21 | 2.060 | 2.11 |
| | 30 | 0.447 | 0.506 |
| WildType Mice | 0.25 | 56.73 | 14.5 |
| | 1 | 120.8 | 6.26 |
| | 2 | 131.2 | 7.54 |
| | 3 | 125.7 | 7.46 |
| | 6 | 101.9 | 11.4 |
| | 9 | 75.94 | 7.06 |
| | 16 | 42.61 | 16.1 |
| | 21 | 27.75 | 16.9 |
| | 30 | 15.52 | 13.0 |

Abbreviations:
Time = Time in days post single-dose injection; d = Day of study; SD = Standard Deviation.

TABLE 26
Summary of Pharmacokinetic Parameters

| | | H4H9816P2 | |
|---|---|---|---|
| Parameter | Units | TrkB^{hu/hu} Mice | WildType Mice |
| Cₘₐₓ | μg/mL | 135 ± 15 | 131 ± 7.5 |
| Tₘₐₓ | d | 1.4 ± 0.56 | 2.0 ± 0 |
| T_{1/2} | d | 2.94 ± 1.1 | 8.36 ± 3.9 |
| AUCₗₐₛₜ | d · μg/mL | 1020 ± 150 | 1730 ± 310 |

PK parameters were derived from mean concentration versus time profiles.
T_{1/2} and AUCₗₐₛₜ are based on concentrations out to day 30.
Abbreviations:
Cₘₐₓ = Peak concentration;
AUC = Area under the concentration-time curve;
AUCₗₐₛₜ = AUC computed from time zero to the time of the last positive concentration;
T_{1/2} = Terminal half-life of elimination;
Tₘₐₓ = the time after administration of antibody when the maximum serum concentration is reached

### Example 12: Ability of Anti-Mouse TrkB Monoclonal Antibodies to Block Interaction between Mouse or Rat TrkB and its Ligand BDNF (Brain Derived Neurotrophic Factor)

Anti-mouse TrkB monoclonal antibodies (mAbs) were generated by immunizing TrkB humanized mice with mouse TrkB protein. The three lead mAbs identified from this immunization are; M2aM14173N, M2aM14178N and M2aM14179N. Lead mAbs of the invention were characterized for their ability to block interaction of mouse or rat TrkB to plate-bound BDNF in a blocking ELISA.

Experiments were carried out using the following procedure. Human BDNF was coated at a concentration of 0.5 g/mL (for blocking mouse TrkB.hFc interaction) or 0.3 μg/mL (for blocking rat TrkB.mmh interaction) in PBS on 96-well microtiter plate and incubated overnight at 4° C. Nonspecific binding sites were subsequently blocked using a 5%(w/v) solution of BSA in PBS (assay buffer). In a 96-well dilution plate, 850 pM mouse TrkB.hFc or rat TrkB.mmh was mixed with the three-fold serially diluted anti-mouse TrkB antibodies and control antibody. The final antibody concentrations ranged from 1.69 pM to 100 nM. The protein-antibody mix was incubated at room temperature (RT) for 1 hour. The pre-bound mix was then transferred in duplicates to microtiter plates coated with BDNF. A control containing assay buffer alone was included to calculate the baseline of the assay. The ELISA plates were incubated at RT for 1 hour and then washed with plate washing solution. Plate-bound mouse TrkB.hFc was detected with HRP-conjugated goat anti-human Fcγ fragment specific antibody (Jackson Immunoresearch) and rat TrkB.mmh was detected with HRP-conjugated anti-histidine antibody (Qiagen). The plates were incubated with detection antibody for 1 hour at RT and then washed with plate washing solution. The assay plates were developed with TMB colorimetric substrates according to the manufacturer's recommended procedure.

The absorbance at 450 nm for each well was recorded and plotted as a function of the concentration of antibody. Data was analyzed in GraphPad Prism software using a four-parameter logistic equation over an 11-point dose response curve and IC₅₀ values were calculated. The calculated IC₅₀ value, defined as the concentration of antibody required to reduce 50% of TrkB binding to BDNF, was used as an indicator of blocking potency. Percent blockade at maximum concentration of the antibody tested was calculated as an indicator of the ability of the antibodies to block the binding of TrkB to BDNF on the plate relative to the baseline of the assay. Binding signal of 850 pM mouse or rat TrkB in absence of the antibody was defined as 100% binding or 0% blocking. The baseline signal of assay buffer alone was defined as 0% binding or 100% blocking.

### Results Summary and Conclusions

The ability of anti-mouse TrkB antibodies to block mouse or rat TrkB binding to BDNF was assessed using blocking ELISAs.

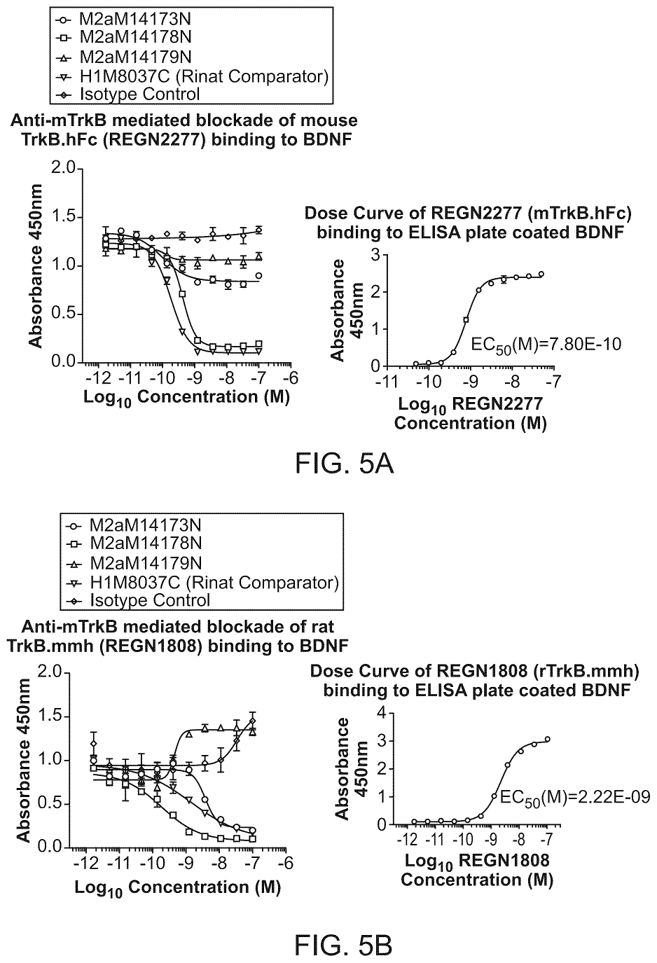

The blocking results are summarized in Table 27 and in FIG. 5A and B. The % blockade is reported for all antibodies and calculated at the highest antibody concentration (100 nM) tested. IC₅₀ values are shown only for antibodies blocking >50% of mouse or rat TrkB binding to BDNF. Out of the three antibodies of this invention, anti-mouse TrkB mAb, M2aM14178N blocked >50% of both mouse and rat TrkB protein binding to BDNF. M2aM14178N blocked binding of 850 pM mouse TrkB.hFc with an IC₅₀ of 426 pM and % blockade of 84.4%. M2aM14178N blocked 850 pM rat TrkB.mmh binding to BDNF with an IC₅₀ value of 184 pM and % blockade of 89.5%. M2aM14173N showed 29.7% blocking of 850 pM mouse TrkB.hFc binding to BDNF. M2aM14173N showed 80.7% blocking of 850 pM rat TrkB.mmh binding to BDNF with an IC₅₀ value of 3.81 nM. M2aM14179N blocked 11.6% of mouse TrkB.hFc binding to BDNF. M2aM14179N showed an increase in rat TrkB.mmh binding to BDNF at concentrations greater than 1 nM.

The comparator anti-mouse TrkB mAb, H1M8037C, blocked 850 pM mouse TrkB.hFc binding to BDNF with an IC₅₀ value of 180 pM and % blockade of 91.5%. H1M8037C blocked 850 pM rat TrkB.mmh binding with an IC₅₀ value of 1.42 nM and % blockade of 83.3%. The mIgG2a isotype control mAb, REGN1097, did not show any blocking of mouse or rat TrkB, under identical assay conditions. At concentrations greater than 10 nM, REGN1027 showed an increase of rat TrkB binding.

TABLE 27
Summary of IC₅₀ (M) values for Anti-mouse TrkB
Blockade of mouse or rat TrkB Binding to BDNF

| | Ab blocking of 780 pM | | Ab blocking of 780 pM | |
|---|---|---|---|---|
| | mouse TrkB.hFc binding | | rat TrkB.mmh binding | |
| | to plate-coated BDNF | | to plate-coated BDNF | |
| | | % Blockade | | % Blockade |
| | IC₅₀ | with 100 nM | IC₅₀ | with 100 nM |
| AbPID | [M] | antibody | [M] | antibody |
| M2aM14173N | not | 29.7 | 3.81E−09 | 80.7 |
| | calculated | | | |
| M2aM14178N | 4.26E−10 | 84.4 | 1.84E−10 | 89.5 |
| M2aM14179N | not | 11.6 | not | −38.1 |
| | calculated | | calculated | |
| H1M8037C | 1.80E−10 | 91.5 | 1.42E−09 | 83.3 |
| (Comparator) | | | | |
| Negative | not | No | not | −9.33 |
| Isotype control | calculated | blocking | calculated | |
| (REGN1097) | | | | |

100% Blockade = OD450 nm value of wells with HRP-conjugated secondary antibody in assay buffer alone (no mouse or rat TrkB protein).
0% Blockade = OD450 nm value of wells with HRP-conjugated secondary antibody in assay buffer in presence of mouse or rat TrkB protein (no TrkB antibody).
Negative Max Blocking % indicate an increase of TrkB binding detected in the presence of antibody.
not calculated = IC₅₀ values not quantitative for antibodies blocking <50% at the highest concentration tested.

### Example 13. Ability of Anti-Human TrkB Monoclonal Antibodies to Block the Interaction between Human TrkB and its Natural Ligands Human BDNF and NT4

The ability of anti-human TrkB antibodies, designated as H4H9814P, H4H9816P2 and H4H9780P, to block TrkB protein binding to plate captured BDNF or NT-4 was measured using two competition sandwich ELISAs. In the assays, various concentrations of anti-TrkB antibody were pre-mixed with a constant amount of dimeric TrkB protein and the reduction of the TrkB binding to the plate immobilized BDNF or NT-4, due to the presence of the antibody, was calculated.

The recombinant dimeric TrkB protein used in the experiments was comprised of a portion of the human TrkB extracellular domain (aa Cys32-His430) expressed with the Fc portion of the human IgG1 at the c-terminus (hTrkB-hFc; Accession #NP_006171.2, molecular weight 69,700 daltons). The BDNF and NT-4 proteins were comprised of the extracellular domain of human BDNF (aa His129-Arg247, Accession #P23560, R&D Systems) or NT-4 (aa Gly81-Ala210, Accession #P34130, R&D Systems), respectively. Two isotype antibody controls, an anti-Fel d 1 human IgG4 antibody, and an antibody specific to a-Fel d 1 antibody with mouse IgG1, were included as controls for IgG background detection.

Experiments were carried out using the following procedure. Human BDNF or NT-4 were separately coated at a concentration of 0.5 μg/mL or 2 μg/mL, respectively, in PBS on a 96-well microtiter plate overnight at 4° C. Nonspecific binding sites were subsequently blocked using BSA solution in PBS. Blocking solution and dilution buffer contained 5% (w/v) solution of BSA in PBS for assay with BDNF coat, or 0.5% (w/v) solution of BSA in PBS for assay with NT-4 coat. On separate microtiter plates, a constant amount of 500 pM of hTrkB-hFc protein was added to serial dilutions of antibodies with final concentrations ranging from 1.7 pM to 100 nM, and solutions with no antibody present. (The constant concentration of hTrkB-hFc for antibody inhibition assays was selected from the approximate midway point within the linear portion of individual binding curves of hTrkB-hFc to plate-coated hBDNF or hNT-4). After one hour incubation at room temperature, antibody-protein complexes with 500 pM constant concentration of hTrkB-hFc protein were transferred to microtiter plates coated with hBDNF or hNT-4. After one hour incubation at room temperature, the wells were washed, and plate-bound hTrkB-hFc was detected with anti-human Fcγ fragment specific goat polyclonal antibodies conjugated with horseradish peroxidase (JacksonImmunoResearch). The plate was then developed using TMB substrate solution (BD Biosciences) according to manufacturer's recommendation and absorbance at 450 nm was measured on a Victor plate reader (PerkinElmer™).

Data analysis was performed using a sigmoidal dose-response model within Prism™ software (GraphPad). The calculated IC₅₀ value, defined as the concentration of antibody required to reduce 50% of hTrkB-hFc binding to hBDNF or hNT-4, was used as an indicator of blocking potency. Percent blockade at maximum concentration of the antibody tested was calculated as an indicator of the ability of the antibodies to block the binding of 500 pM of hTrkB-hFc to hBDNF or hNT-4 on the plate, relative to the baseline of the assay. In the calculation, binding signal of the sample of 500 pM of hTrkB-hFc without the presence of the antibody was referenced as 100% binding or 0% blocking; and the baseline signal of the sample of buffer without hTrkB-hFc or the antibody was referenced as 0% binding or 100% blocking.

### Results Summary and Conclusions

The ability of anti-TrkB antibodies to block TrkB binding to BDNF or NT-4 was assessed using two competition sandwich ELISAs. Human TrkB-hFc binding to hBDNF or hNT-4 coated on 96-well microtiter plates, in the presence of serially diluted antibodies or no antibody controls, were detected with HRP-conjugated anti-human Fcγ fragment specific goat polyclonal antibodies. IC50 values were calculated and used as the potency indicator of antibody blocking hTrkB-hFc binding to hBDNF or hNT-4. In addition, the maximum blockade of 500 pM hTrkB-hFc with each antibody at the highest tested concentration was calculated and compared.

The blocking results are summarized in Table 28. The % blockade is reported for all antibodies and calculated at the highest tested antibody concentration of 100 nM. Negative % blockade indicates an increase of TrkB binding detected in the presence of antibody. IC50 values are shown for antibodies blocking >50% of TrkB binding to BDNF or NT-4. IC50 values are not quantitative for antibodies blocking <50% and was reported as (−).

At the highest concentration of antibody tested, one (H4H9780P) of the three anti-TrkB antibodies blocked >50% hTrkB binding to BDNF or NT-4 ligands with IC50 values of 150 pM and 180 pM, respectively, with percent blockade at 100 nM antibody of 93% for BDNF and 80% for NT-4. At 3.7 nM, this antibody blocked 500 pM hTrkB-hFc binding to NT-4 with 99%. The decrease of % blockade at the highest tested concentrations may be attributed to H4H9780P non-specific binding to the microtiter plate and detection of this binding with HRP-conjugated anti-human Fcγ fragment specific polyclonal antibodies.

Two of the three anti-TrkB antibodies (H4H9814P and H4H9816P2) and irrelevant blocking control antibodies blocked <50% of hTrkB binding to BDNF or NT-4. The comparator blocked 500 pM hTrkB-hFc binding >50% to both BDNF and NT-4.

TABLE 28

| | Ab blocking 500 pM | | Ab blocking 500 pM | |
|---|---|---|---|---|
| | hTrkB-hFc binding | | hTrkB-hFc binding | |
| | to plate-coated hBDNF | | to plate-coated hNT-4 | |
| | | % Blockade | | % Blockade |
| | IC50 | with 100 nM | IC50 | with 100 nM |
| Ab PID | (M) | antibody | (M) | antibody |
| H4H9814P | — | 6 | — | 38 |
| H4H9816P2 | — | −56 | — | −123 |
| H4H9780P | 1.5E−10 | 93 | 1.8E−10 | 80* |

CONTROLS

| | | | | |
|---|---|---|---|---|
| a-TrkB-mIgG1 | 1.7E−10 | 97 | 4.5E−11 | 95 |
| (H1M8037C | | | | |
| comparator) | | | | |
| hIgG4 negative | — | −31 | — | −45 |
| isotype control | | | | |
| mIgG1 negative | — | −19 | — | −14 |
| isotype control | | | | |

*Blocked 99% at 3.7 nM H4H9780P antibody concentration.

### Example 14. Octet Cross-Competition between Different Anti-hTrkB Monoclonal Antibodies

To assess whether two antibodies compete with one another for binding to their epitopes on hTrkB-mmh, binding competition between anti-hTrkB monoclonal antibodies was determined using a real time, label-free bio-layer interferometry assay on an Octet RED384 biosensor (Pall ForteBio Corp.). Cross competition experiments were performed at 25° C. in 0.01 M HEPES pH7.4, 0.15M NaCl, 3.4 mM EDTA, 0.05% v/v Surfactant Tween-20, 0.1 mg/mL BSA (HBS-EP buffer) with the plate shaking at the speed of 1000 rpm. All anti-hTrkB antibody and hTrkB-mmh solutions tested were prepared in Octet HBS-EP buffer. To assess whether 2 antibodies were able to compete with one another for binding to their respective epitopes on hTrkB-mmh, approximately ˜0.14-0.24 nm of hTrkB-mmh was first captured on anti-His coated Octet biosensor tips from wells containing 50 μg/mL of hTrkB-mmh for 5 minutes. The hTrkB-mmh captured Octet biosensor tips were saturated by submerging for 5 minutes into wells containing 50 ug/ml of the first anti-hTrkB monoclonal antibody (hereby referred to as mAb-1), followed by submerging in wells containing the second anti-HTrkB monoclonal antibody (hereby referred to as mAb-2) for an additional 5 minutes. Between steps, the Octet biosensor tips were washed in HBS-EP buffer for 30 seconds.

The real-time binding response was monitored during the course of the experiment and the binding response at the end of every step was recorded. The response of mAb-2 binding to hTrkB.mmh pre-complexed with mAb-1 was compared and competitive/non-competitive behavior of different anti-hTrkB monoclonal antibodies was determined using a 60% inhibition threshold.

Table 29 explicitly defines the relationships of antibodies competing in both directions, independent of the order of binding.

### Results

TABLE 29
Cross-competition of anti-hTrkB antibodies
for binding to human hTrkB.mmh.

| mAb-1 | mAb-2 |
|---|---|
| H4H9814P | H4H9816P2 |
| | H4H9814P |
| H4H9816P2 | H4H9814P |
| | H4H9816P2 |
| H4H9780P | H4H9780P |
| H1M8037C (comparator) | H1M8037C (comparator) |

### Example 15. Biacore Binding Kinetics of Surrogate Anti-Mouse TrkB Monoclonal Antibodies Binding to Different TrkB Reagents Measured at 25° C.

To assess whether two antibodies compete with one another for binding to their epitopes on mTrkB-mmh, binding competition between anti-mTrkB monoclonal antibodies was determined using a real time, label-free bio-layer interferometry assay on an Octet HTX biosensor (Pall ForteBio Corp.). Cross competition experiments were performed at 25° C. in 0.01 M HEPES pH7.4, 0.15M NaCl, 3.4 mM EDTA, 0.05% v/v Surfactant Tween-20, 0.1 mg/mL BSA (HBS-EP buffer) with the plate shaking at the speed of 1000 rpm. All anti-mTrkB antibody and mTrkB-mmh solutions tested were prepared in Octet HBS-EP buffer. To assess whether 2 antibodies were able to compete with one another for binding to their respective epitopes on mTrkB-mmh, approximately ˜0.20 -0.27 nm of mTrkB-mmh was first captured on anti-His coated Octet biosensor tips from wells containing 20 μg/mL of mTrkB-mmh for 5 minutes. The mTrkB-mmh captured Octet biosensor tips were saturated by submerging for 5 minutes into wells containing 50 ug/ml of the first anti-mTrkB monoclonal antibody (hereby referred to as mAb-1), followed by submerging in wells containing the second anti-mTrkB monoclonal antibody (hereby referred to as mAb-2) for an additional 3 minutes. Between steps, the Octet biosensor tips were washed in HBS-EP buffer for 30 seconds.

The real-time binding response was monitored during the course of the experiment and the binding response at the end of every step was recorded. The response of mAb-2 binding to mTrkB.mmh pre-complexed with mAb-1 was compared and competitive/non-competitive behavior of different anti-mTrkB monoclonal antibodies was determined using a 50% inhibition threshold.

Table 30 explicitly defines the relationships of antibodies competing in both directions, independent of the order of binding.

### Results

TABLE 30
Cross-competition of anti-mTrkB antibodies
for binding to mouse hTrkB.mmh.

| mAb-1 | mAb-2 |
|---|---|
| M2aM14173N | M2aM14178N |
| | M2aM14173N |
| M2aM14178N | M2aM14173N |
| | M2aM14178N |
| M2aM14179N | M2aM14179N |
| H1M8037C (comparator) | H1M8037C (comparator) |

### Example 16: Octet Blocking: Blocking of Anti-Human TrkB or Anti-Mouse TrkB Antibodies from Binding to TrkB by BDNF or NT-4

### Experiment 1

Blocking of anti-human TrkB or anti-mouse TrkB antibodies from binding to TrkB by BDNF or NT-4 was evaluated using a real-time bio-layer interferometry (BLI) based Octet HTX instrument. The entire study was performed in 10 mM HEPES pH 7.4, 300 mM NaCl, 3 mM EDTA, 1 mg/mL BSA, 0.02% NaN3 and 0.05% v/v Surfactant Tween-20 (HBS-EBT running buffer) at 25° C. All the samples were dispensed in a 384 tilted well plate and the plate was placed on the orbital shaker with the shake speed of 1000 rpm. hTrkB.mFc was captured on anti-mFc (AMC) Octet sensors while hTrkB.hFc or mTrkB.hFc were captured on anti-hFc (AHC) Octet sensors by dipping in wells containing 10 μg/mL of TrkB reagents for 2 minutes. hTrkB.mFc or hTrkB.hFc captured Octet biosensors were saturated by dipping in wells containing 20 nM of BDNF, hNT-4, or mNT-4 for 2 minutes followed by dipping Octet biosensors in wells containing 300 nM of different TrkB mAbs for 4 minutes. Binding of TrkB mAbs to the complex of TrkB and BDNF, hNT-4, or mNT-4 was determined using Scrubber 2.0c analysis software.

Binding of any anti-human TrkB or anti-mouse TrkB antibodies of this invention was not blocked by both BDNF and NT-4 as reported in Table 31 and Table 32.

TABLE 31
Binding of anti-human TrkB monoclonal antibodies to
the complex of hTrkB-mFc and BDNF or human NT-4.

| | | | Anti-human TrkB antibody Binding (nm) | | |
|---|---|---|---|---|---|
| | Capture | | | BDNF Saturation | hNT-4 Saturation |
| Capture | Level | | | (0.05 ± 0.007) | (0.05 ± 0.006) |
| Surface | (nm) | mAb | No Ligand | nm | nm |
| hTrkB.mFc | 0.29 ± 0.01 | REGN1945 | 0.01 ± 0.01 | 0.00 ± 0.00 | 0.00 ± 0.00 |
| | | (negative isotype control) | | | |
| | | H4H9780P | 0.18 ± 0.01 | 0.20 ± 0.014 | 0.2 ± 0.007 |
| | | H4H9814P | 0.18 ± 0.01 | 0.22 ± 0.006 | 0.23 ± 0.00 |
| | | H4H9816P2 | 0.19 ± 0.00 | 0.26 ± 0.007 | 0.24 ± 0.007 |
| hTrkB.hFc | 0.47 ± 0.01 | H1M8037C (Comparator) | 0.38 ± 0.01 | 0.07 ± 0.00 | 0.10 ± 0.006 |

The values of anti-human TrkB antibodies binding to the complex of hTrkB.mFc or hTrkB.hFc with BDNF or hNT-4 represent an average of binding response measured using 3 independent Octet biosensors along with the standard deviation. Binding of BDNF or hNT-4 to hGFRa3.mFc did not inhibit the binding of any anti-human TrkB antibodies.

TABLE 32
Binding of anti-mouse TrkB monoclonal antibodies to the complex of mTrkB-hFc and BDNF or mouse NT-4.

| | | | Anti-mouse TrkB antibody Binding (nm) | | |
|---|---|---|---|---|---|
| | Capture | | | BDNF Saturation | mNT-4 Saturation |
| Capture | Level | | | (0.07 ± 0.006) | (0.07 ± 0.005) |
| Surface | (nm) | mAb | No Ligand | nm | nm |
| mTrkB.hFc | 0.47 ± 0.01 | REGN1318 | 0.01 ± 0.00 | 0.00 ± 0.00 | 0.00 ± 0.006 |
| | | (negative isotype control) | | | |
| | | M2aM14173N | 0.42 ± 0.006 | 0.38 ± 0.006 | 0.4 ± 0.00 |
| | | M2aM14178N | 0.47 ± 0.00 | 0.35 ± 0.007 | 0.41 ± 0.006 |
| | | M2aM14179N | 0.42 ± 0.006 | 0.44 ± 0.006 | 0.43 ± 0.006 |
| | | H1M8037C (Comparator) | 0.42 ± 0.006 | 0.06 ± 0.006 | 0.11 ± 0.00 |

The values of anti-mouse TrkB antibodies binding to the complex of mTrkB.mFc with BDNF or mNT-4 represent an average of binding response measured using 3 independent Octet biosensors along with the standard deviation. Binding of BDNF or mNT-4 to mGFRa3.hFc did not inhibit the binding of any anti-mouse TrkB antibodies.

### Experiment 2

Blocking of BDNF or NT-4 from binding to TrkB by anti-human TrkB or anti-mouse TrkB antibodies was evaluated using a real-time bio-layer interferometry (BLI) based Octet HTX instrument. The entire study was performed in 10 mM HEPES pH 7.4, 300 mM NaCl, 3 mM EDTA, 1 mg/mL BSA, 0.02% NaN3 and 0.05% v/v Surfactant Tween-20 (HBS-EBT running buffer) at 25° C. All the samples were dispensed in a 384 tilted well plate and the plate was placed on the orbital shaker with the shake speed of 1000 rpm. hTrkB.mFc was captured on anti-mFc (AMC) Octet sensors while hTrkB.hFc or mTrkB.hFc were captured on anti-hFc (AHC) Octet sensors by dipping in wells containing 10 μg/mL of TrkB reagents for 2 minutes. hTrkB.mFc or hTrkB.hFc captured Octet biosensors were saturated by dipping in wells containing 300 nM of different TrkB mAbs for 4 minutes followed by dipping Octet biosensors in wells containing 20 nM of BDNF, hNT-4, or mNT-4 for 2 minutes. Binding of BDNF, hNT-4, or mNT-4 to the complex of TrkB and different TrkB mAbs was determined using Scrubber 2.0c analysis software.

Binding of 1 out of 3 anti-human TrkB antibodies of this invention blocked the binding of BDNF and hNT-4 as reported in Table 33. Binding of 1 out of 3 anti-mouse TrkB antibodies of this invention partially blocked the binding of BDNF and mNT-4 as reported in Table 34.

TABLE 33
Binding of BDNF or human NT-4 to the complex of hTrkB-mFc and anti-human TrkB monoclonal antibodies.

| | | | BDNF Binding | | hNT-4 Binding | |
|---|---|---|---|---|---|---|
| Capture | Capture | | mAb | | mAb | |
| Surface | Level (nm) | mAh | Binding (nm) | BDNF | Binding (nm) | hNT-4 |
| hTrkB.mFc | 0.28 ± 0.01 | No mAb | N/A | 0.05 ± 0.007 | N/A | 0.04 ± 0.005 |
| | | REGN1945(negative | 0.01 ± 0.01 | 0.04 ± 0.01 | 0.01 ± 0.01 | 0.033 ± 0.006 |
| | | isotype control) | | | | |
| | | H4H9780P | 0.18 ± 0.01 | 0.01 ± 0.00 | 0.18 ± 0.01 | 0.007 ± 0.006 |
| | | H4H9814P | 0.18 ± 0.01 | 0.05 ± 0.00 | 0.18 ± 0.01 | 0.04 ± 0.00 |
| | | H4H9816P2 | 0.19 ± 0.00 | 0.057 ± 0.006 | 0.18 ± 0.01 | 0.043 ± 0.006 |
| hTrkB.hFc | 0.48 ± 0.01 | H1M8037C (Comparator) | 0.38 ± 0.01 | −0.017 ± 0.006 | 0.39 ± 0.01 | −0.013 ± 0.006 |

The values of BDNF or hNT-4 binding to the complex of hTrkB.mFc or hTrkB.hFc with anti-human TrkB antibodies represent an average of binding response measured using 3 independent Octet biosensors along with the standard deviation. Binding of H4H9780P to hGFRa3.mFc blocked the binding of BDNF and hNT-4.

TABLE 34
Binding of BDNF or mouse NT-4 to the complex of mTrkB-hFc and anti-mouse TrkB monoclonal antibodies.

| | | | BDNF Binding | | hNT-4 Binding | |
|---|---|---|---|---|---|---|
| Capture | Capture | | mAb | | mAb | |
| Surface | Level (nm) | mAh | Binding (nm) | BDNF | Binding (nm) | hNT-4 |
| mTrkB.hFc | 0.48 ± 0.01 | No mAb | N/A | 0.07 ± 0.01 | N/A | 0.07 ± 0.005 |
| | | REGN1318 (negative | 0.01 ± 0.00 | 0.06 ± 0.00 | 0.01±0.00 | 0.06 ± 0.00 |
| | | isotype control) | | | | |
| | | M2aM14173N | 0.42 ± 0.006 | 0.07 ± 0.00 | 0.413 ± 0.006 | 0.06 ± 0.00 |
| | | M2aM14178N | 0.47 ± 0.00 | 0.05 ± 0.00 | 0.47 ± 0.01 | 0.033 ± 0.006 |
| | | M2aM14179N | 0.42 ± 0.006 | 0.07 ± 0.00 | 0.41±0.00 | 0.06 ± 0.00 |
| | | H1M8037C (Comparator) | 0.42 ± 0.006 | −0.017 ± 0.006 | 0.40 ± 0.00 | −0.02 ± 0.00 |

The values of BDNF or mNT-4 binding to the complex of mTrkB.hFc and anti-mouse TrkB antibodies represent an average of binding response measured using 3 independent Octet biosensors along with the standard deviation. Binding of M2aM14178N to mTrkB.hFc partially blocked the binding of BDNF and mNT-4.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

We claim:

1. An isolated antibody or antigen-binding fragment thereof that binds specifically to tropomyosin receptor kinase B (TrkB), wherein the antibody or antigen-binding fragment thereof comprises three heavy chain complementarity determining regions (CDRs) (HCDR1, HCDR2 and HCDR3) contained within a heavy chain variable region (HCVR) comprising an amino acid sequence selected from the group consisting of SEQ ID NOs: 2, 18, 34, 49, 59 and 68; and
three light chain CDRs (LCDR1, LCDR2 and LCDR3) contained within a light chain variable region (LCVR) comprising an amino acid sequence selected from the group consisting of SEQ ID NOs: 10, 26, 42, 53, 63 and 72.

2. The isolated antibody or antigen-binding fragment of claim 1, wherein the antibody or antigen-binding fragment thereof comprises the CDRs of a HCVR/LCVR amino acid sequence pair selected from the group consisting of SEQ ID NOs: 2/10, 18/26, 34/42, 49/53, 59/63 and 68/72.

3. The isolated antibody or antigen-binding fragment thereof of claim 1, comprising an HCVR/LCVR amino acid sequence pair selected from the group consisting of SEQ ID NOs: 2/10, 18/26, 34/42, 49/53, 59/63 and 68/72.

4. The isolated antibody or antigen-binding fragment thereof of claim 1, comprising:
(a) a HCDR1 domain having an amino acid sequence selected from the group consisting of SEQ ID NOs: 4, 20, 36, 50, 60 and 69;
(b) a HCDR2 domain having an amino acid sequence selected from the group consisting of SEQ ID NOs: 6, 22, 38, 51, 61 and 70;
(c) a HCDR3 domain having an amino acid sequence selected from the group consisting of SEQ ID NOs: 8, 24, 40, 52, 62 and 71;
(d) a LCDR1 domain having an amino acid sequence selected from the group consisting of SEQ ID NOs: 12, 28, 44, 54, 64 and 73;
(e) a LCDR2 domain having an amino acid sequence selected from the group consisting of SEQ ID NOs: 14, 30, 46, 55, 65 and 74; and
(f) a LCDR3 domain having an amino acid sequence selected from the group consisting of SEQ ID NOs: 16, 32, 48, 56, 66 and 75.

5. The isolated antibody or antigen-binding fragment of claim 1, wherein the antibody or antigen-binding fragment thereof comprises a set of six CDRs (HCDR1-HCDR2-HCDR3-LCDR1-LCDR2-LCDR3) selected from the group consisting of: (a) SEQ ID NOs: 4-6-8-12-14-16; (b) SEQ ID NOs: 20-22-24-28-30-32; (c) SEQ ID NOs: 36-38-40-44-46-48; (d) SEQ ID NOs: 50-51-52-54-55-56; (e) SEQ ID NOs: 60-61-62-64-65-66; and (f) SEQ ID NOs: 69-70-71-73-74-75.

6. The isolated antibody or antigen-binding fragment of claim 1, wherein the antibody or antigen-binding fragment thereof binds human TrkB with a K_{D} of less than about 300 nM as measured by surface plasmon resonance at 25° C. or 37° C.

7. The isolated antibody or antigen-binding fragment of claim 6, wherein the antibody or antigen-binding fragment thereof binds human TrkB with a K_{D} selected from the group consisting of less than about 150 nM, less than about 50 pM, less than about 100 pM as measured by surface plasmon resonance at 25° C. or 37° C.

8. The isolated antibody or antigen-binding fragment of claim 1, wherein the antibody or antigen-binding fragment thereof binds human TrkB with a dissociative half life (t½) selected from the group consisting of greater than about 10 minutes, greater than about 40 minutes, greater than about 120 minutes as measured by surface plasmon resonance at 25° C. or 37° C.

9. The isolated antibody or antigen-binding fragment of claim 1, wherein the antibody or antigen-binding fragment thereof activates human TrkB signaling in the absence of BDNF in cells engineered to express TrkB, with an EC₅₀ of less than about 100 pM.

10. The isolated antibody or antigen-binding fragment of claim 1, wherein the antibody or antigen-binding fragment thereof enhances activation of human TrkB signaling in the presence of BDNF in cells engineered to express TrkB, with an EC₅₀ of less than about 100 pM.

11. The isolated antibody or antigen-binding fragment thereof of claim 1, which when injected into the hippocampus of humanized TrkB mice, demonstrates TrkB activation, as shown by an increase in TrkB phosphorylation.

12. The isolated antibody or antigen-binding fragment of claim 1, wherein the antibody or antigen-binding fragment thereof demonstrates activation of the MAPK/ERK and PI3K/Akt signaling pathways, as demonstrated following incubation of primary mouse cortical neurons with an agonist anti-TrkB antibody.

13. The isolated antibody or antigen-binding fragment of claim 1, wherein the antibody or antigen-binding fragment thereof enhances survival of retinal ganglion cells as shown in an optic nerve transection model in TrkB humanized rats.

14. The isolated antibody or antigen-binding fragment of claim 1, wherein the antibody or antigen-binding fragment thereof promotes weight loss in humanized TrkB mice.

15. The isolated antibody or antigen-binding fragment of claim 1, wherein the antibody or antigen-binding fragment thereof promotes a loss of fat mass in humanized TrkB mice.

16. The isolated antibody or antigen-binding fragment of claim 1, wherein the antibody or antigen-binding fragment thereof promotes a decrease in food and water consumption in humanized TrkB mice.

17. The isolated antibody or antigen-binding fragment of claim 1, wherein the antibody or antigen-binding fragment thereof promotes an increase in locomotor activity in humanized TrkB mice.

18. The isolated antibody or antigen-binding fragment thereof of claim 1, wherein the antibody or antigen-binding fragment thereof exhibits one or more properties selected from the group consisting of:
(a) is an agonist antibody;
(b) binds human TrkB with a K_{D} of less than about 200 nM as measured by surface plasmon resonance at 25° C. or at 37° C.;
(c) binds human TrkB with a dissociative half life (t½) of greater than about 10 minutes as measured by surface plasmon resonance at 25° C. or at 37° C.;
(d) activates human TrkB signaling in the absence of brain derived neurotrophic factor (BDNF) in cells engineered to express human TrkB with an EC₅₀ of less than about 100 pM;
(e) enhances TrkB phosphorylation when injected into the hippocampus of mice homozygous for human TrkB receptor (TrkB^{hu/hu} );
(f) promotes weight loss when injected into mice homozygous for human TrkB receptor (TrkB^{hu/hu} );
(g) increases retinal ganglion cell (RGC) survival as assessed in an optic nerve transection model in humanized TrkB rats;
(h) activates the MAPK/ERK and PI3K/Akt signaling pathways;
(i) enhances/increases the survival of neuronal cells in vitro; and
(j) blocks the binding of TrkB to BDNF and/or NT-4 with an IC₅₀ of less than 5 nM.

19. A pharmaceutical composition comprising the antibody or antigen-binding fragment of claim 1, and a pharmaceutically acceptable carrier or diluent.

20. An isolated nucleic acid molecule comprising a polynucleotide sequence that encodes an anti-TrkB antibody or fragment thereof according to claim 1.

21. A vector comprising the polynucleotide sequence of claim 20.

22. A cell expressing the vector of claim 21.

23. A method for enhancing a biological activity mediated by TrkB, the method comprising:
contacting TrkB with a biologically effective amount of an agonist anti-TrkB antibody of claim 1, or contacting TrkB with a pharmaceutical composition containing a biologically effective amount of an agonist anti-TrkB antibody of claim 1.

24. The method of claim 23, wherein the biological activity is neuronal protection or neuronal survival and neuronal protection or neuronal survival is enhanced upon contact of TrkB with an agonist anti-TrkB antibody.

25. The method of claim 23, wherein contacting TrkB with an agonist anti-TrkB antibody results in neuroprotection and survival of retinal ganglion cells (RGCs).

26. A method of treating or preventing a disease or disorder associated with TrkB activity or expression, or for ameliorating at least one symptom associated with the disease or disorder associated with TrkB activity or expression, the method comprising administering a therapeutically effective amount of an agonist anti-TrkB antibody of claim 1, or administering a pharmaceutical composition containing a therapeutically effective amount of an agonist anti-TrkB antibody of claim Ito a subject in need of such treatment.

27. The method of claim 26, wherein the disease or disorder is a disease or disorder of the eye selected from the group consisting of glaucoma, diabetic retinopathy, age-related macular degeneration, ischemic optic neuropathy, optic neuritis, retinal ischemia, photoreceptor degeneration, retinitis pigmentosa and retinal artery or vein occlusions.

28. The method of claim 27, wherein the disease or disorder is glaucoma.

29. A method for achieving a reduction of body weight in a subject, the method comprising administering a TrkB agonist, wherein the TrkB agonist is an antibody or antigen-binding fragment thereof specific for TrkB according to claim 1, or a pharmaceutical composition comprising the antibody or antigen-binding fragment thereof, to the subject.

30. A method for achieving a reduction of fat mass in a subject, the method comprising administering a TrkB agonist, wherein the TrkB agonist is an antibody or antigen-binding fragment thereof specific for TrkB according to claim 1, or a pharmaceutical composition comprising the antibody or antigen-binding fragment thereof, to the subject.

31. A method for promoting neuronal survival in a subject, the method comprising administering a therapeutically effective amount of a TrkB agonist, wherein the TrkB agonist is an antibody or antigen-binding fragment thereof specific for TrkB according to claim 1, or a pharmaceutical composition comprising a therapeutically effective amount of the antibody or antigen-binding fragment thereof. to the subject.

32. The method of claim 23, wherein contacting TrkB is by injecting a patient with an injectable formulation.
